SEQUENCE LISTING (45 sequences)

SEQ ID NO: 1 - protein, 297 aa, Pseudomonas sp.
  REGION 1..297: note = alkane 1-mono- oxygenase AlkB
MSTVTPKDIQFRKLMDAPLLAWPTMLLFFGSLGTLGLVDYHAVAGNIPLWVGTIINGLVIYLLFSCVHDASHGAVSRIKWINELPGHIGMFFFGPIAPFNVARFIHMQHHRFTNDSTMDPDHFGHKMDWLFPLRWSNFDYYYTKYFFMRAPAAMRKKFLPRLIIHFSVIIGVLIALTLAGYGLEALMLWILPTRISSFLFVMAFVYLPHEPFQSTAKEDEYRASNIRPGAEWLLTPLLAYQNYHLMHHLYPTAPFYNMIKLWELRHAEHMANSPLIVKTFGLQPLDEHARHAGRDAA

SEQ ID NO: 2 - protein, 52 aa, Pseudomonas sp.
  REGION 1..52: note = ruberodoxin peptide
MKRWQCRYCSFIYDEALGLPAEGIAPGTPLSDVPDDWMCPDCGATKVDFDEI

SEQ ID NO: 3 - protein, 384 aa, Pseudomonas sp.
  REGION 1..384: note = rubredoxin reductase
MSENAPLVIIGTGLAGYNLAREWRKLDAQSPLLLITADDGRSYSKPMLSTGFSKNKDADGLAMAEPGAMAEQLKARILTHTRVTGIDPGHKRIWIGEEQVEYRDLVLAWGAETIRVPVQGDALDAIFPINDLEDYARFRAAAAGKRRVLLLGAGLIGCEFANDLSAGGYQVDVVAPCEQVMPGLLHPAAAAAVQAGLEGLGVRFHLGPVLNRLERAGESLEAHLSDGQAIPCDLVVSAVGLRPRIDLAAEAGLEVNRGVVVNRELRTSHANIYALGDCAEVDGLNLLYVMPLMSCARALAQTLAGKPTAVSYGPMPVTVKTPVCPLVVSPPPRGSDGQWLVEGSGADLKVLCRDTAGRLLGYALTGAAVSEKLSLNKELPALLA

SEQ ID NO: 4 - DNA, 2285 bp, synthetic construct
  source 1..2285: mol_type = other DNA
  misc_feature 1..2285: note = Isolated artificial sequences for alkane hydroxylase system
tctagaaataattttgtttaactttaagaaggagatataccatgtcgaccgtgaccccgaaggacatccagttccgcaagctcatggacgccccgctcctcgcctggccgaccatgctcctcttcttcggctcgctcggcaccctcggcctcgtggactaccacgccgtggccggcaacatcccgctctgggtgggcaccatcatcaacggcctcgtgatctacctcctcttctcgtgcgtgcacgacgcctcgcacggcgccgtgtcgcgcatcaagtggatcaacgagctcccgggccacatcggcatgttcttcttcggcccgatcgccccgttcaacgtggcccgcttcatccacatgcagcaccaccgcttcaccaacgactcgactatggacccggaccacttcggccacaagatggactggctcttcccgctccgctggtcgaacttcgactactactacaccaagtacttcttcatgcgcgccccggccgccatgcgcaagaagttcctcccgcgcctcatcatccacttctcggtgatcatcggcgtgctcatcgccctcaccctcgccggctacggccttgaggccctcatgctctggattctcccgacccgcatctcgtcgttcctcttcgtgatggccttcgtgtacctcccgcacgagccgttccagtcgaccgccaaggaggacgagtaccgcgcctcgaacatccgcccgggcgccgagtggctcctcaccccgctcctcgcctaccagaactaccacctcatgcaccacctctacccgaccgccccgttctacaacatgatcaagctctgggagctccgccacgccgagcacatggccaactcgccgctcatcgtgaagaccttcggcctccagccgctcgacgagcacgcccgccacgccggccgcgacgccgcctaaggaggtgctgccatgaagcgctggcagtgccgctactgctcgttcatctacgacgaggccctcggcctcccggccgagggcatcgccccgggcaccccgctctcggacgtgccggacgactggatgtgcccggactgcggcgccaccaaggtggacttcgacgagatctaaggaggtaactcaatgtctgaaaacgctccgctggttatcatcggtaccggtctggctggttacaacctggctcgtgaatggcgtaaactggacgctcagtctccgctgctgctgatcaccgctgacgacggtcgttcttactctaaaccgatgctgtctaccggtttctctaaaaacaaagacgctgacggtctggctatggctgaaccgggtgctatggctgaacagctgaaagctcgtatcctgacccacacccgtgttaccggtatcgacccgggtcacaaacgtatctggatcggtgaagaacaggttgaataccgtgacctggttctggcttggggtgctgaaaccatccgtgttccggttcagggtgacgctctggacgctatcttcccgatcaacgacctggaagactacgctcgtttccgtgctgctgctgctggtaaacgtcgtgttctgctgctgggtgctggtctgatcggttgcgaattcgctaacgacctgtctgctggtggttaccaggttgacgttgttgctccgtgcgaacaggttatgccgggtctgctgcacccggctgctgctgctgctgttcaggctggtctggaaggtctgggtgttcgtttccacctgggtccggttctgaaccgtctggaacgtgctggtgaatctctggaagctcacctgtctgacggtcaggctatcccgtgcgatctcgttgtaagcgctgttggtctgcgtccgcgtatcgacctggctgctgaagctggtctggaagttaaccgtggtgttgttgttaaccgtgaactgcgtacctctcacgctaacatctacgctctgggtgactgcgctgaagttgacggtctgaacctgctgtacgttatgccgctgatgtcttgcgctcgtgctctggctcagaccctggcgggtaaaccaaccgctgtaagctacggtccgatgccggttaccgttaaaaccccggtttgcccgctggttgtttctccgccgccgcgtggttctgacggtcagtggctggttgaaggttctggtgctgacctgaaagttctgtgccgtgacaccgctggtcgtctgctgggttacgctctgaccggtgctgctgtttctgaaaaactgtctctgaacaaagaactgccggctctgctggcttaattaagcggccgc

SEQ ID NO: 5 - DNA, 2135 bp, Artemisia annua
  source 1..2135: mol_type = unassigned DNA
  misc_feature 1..2135: note = Beta-farnesene synthase
gaattcgattatgtggtgagggaattaccctgtattagtgcaacggcgaggaacgaaactggcgcggacatgattttgattgataattgaatgttgcggcttctacaattttcacaattatgtgagtattgtaagcaatcatgacatgataaattctgataacgtgtcgggtcatgtattcagccagtatgaggagcgccttcagaaatttatttgctttgtgagcggataacaattattagattcaccggcgagccagcaggaatttcactctagatgacaggagggacatatgtcgaccctgccgatctcgtcggtgtcgttctcgtcgtcgacctcgccgctcgtggtggacgacaaggtgtcgaccaagcccgacgtgatccgccatacgatgaacttcaacgcctcgatctggggcgaccagttcctgacgtatgacgagccggaggacctcgtgatgaagaagcagctcgtggaggagctcaaggaagaggtgaagaaggagctgatcaccatcaagggctcgaacgagcccatgcagcacgtgaagctcatcgagctgatcgacgcggtgcagcgcctgggcatcgcgtaccatttcgaggaagagatcgaggaagccctccagcacatccatgtgacctatggcgagcagtgggtggacaaggagaacctgcagtcgatctcgctgtggttccgcctgctccgccagcagggcttcaacgtgtcgtcgggcgtgttcaaggacttcatggacgagaagggcaagttcaaggagtcgctctgcaacgacgcccagggcatcctggccctctatgaggccgcgttcatgcgcgtggaggacgagacgatcctcgacaacgccctggagttcacgaaggtgcacctggacatcatcgcgaaggacccgtcgtgcgactcgtcgctccgcacgcagatccatcaggccctgaagcagcccctgcgccgccgcctggcccgcatcgaggcgctgcactacatgcccatctatcagcaggagacgagccatgacgaggtgctgctcaagctcgcgaagctggacttctcggtgctgcagtcgatgcacaagaaggagctctcgcatatctgcaagtggtggaaggacctggacctgcagaacaagctgccctatgtgcgcgaccgcgtggtggagggctatttctggattctgtcgatctactatgagccgcagcatgcccgcacccgcatgttcctgatgaagacgtgcatgtggctcgtggtgctggacgacaccttcgacaactacggcacgtatgaggagctggagatcttcacccaggcggtggagcgctggtcgatctcgtgcctcgacatgctgccggagtacatgaagctcatctatcaggagctcgtgaacctgcacgtggagatggaggagtcgctggagaaggaaggcaagacgtaccagatccattatgtgaaggagatggccaaggagctggtgcgcaactacctcgtggaggcgcgctggctgaaggaaggctacatgcccaccctcgaggagtatatgtcggtgtcgatggtgaccggcacgtacggcctcatgatcgcccgctcgtatgtgggccgcggcgacatcgtgacggaggacacgttcaagtgggtgtcgtcgtatccgcccatcatcaaggcctcgtgcgtgatcgtgcgcctgatggacgacatcgtgtcgcacaaggaagagcaggagcgcggccatgtggcgtcgtcgatcgagtgctactcgaaggagtcgggcgcctcggaggaagaggcctgcgagtatatctcgcgcaaggtggaggacgcctggaaggtgatcaaccgcgagtcgctgcgcccgaccgccgtgccgttccccctgctcatgcccgccatcaacctggcgcgcatgtgcgaggtgctctattcggtgaacgacggcttcacccatgcggagggcgacatgaagtcgtatatgaagtcgttcttcgtccacccgatggtcgtgtgatactagaggagttggtagctcttgatccggcaaacaaaccaccgttggtagcggtggtttttttgtttgcaagcagcagattacgcgcagaaaaaaaggtactagagagtgcaagctt

SEQ ID NO: 6 - protein, 574 aa, Artemisia annua
  REGION 1..574: note = Beta-farnesene synthase
MSTLPISSVSFSSSTSPLVVDDKVSTKPDVIRHTMNFNASIWGDQFLTYDEPEDLVMKKQLVEELKEEVKKELITIKGSNEPMQHVKLIELIDAVQRLGIAYHFEEEIEEALQHIHVTYGEQWVDKENLQSISLWFRLLRQQGFNVSSGVFKDFMDEKGKFKESLCNDAQGILALYEAAFMRVEDETILDNALEFTKVHLDIIAKDPSCDSSLRTQIHQALKQPLRRRLARIEALHYMPIYQQETSHDEVLLKLAKLDFSVLQSMHKKELSHICKWWKDLDLQNKLPYVRDRVVEGYFWILSIYYEPQHARTRMFLMKTCMWLVVLDDTFDNYGTYEELEIFTQAVERWSISCLDMLPEYMKLIYQELVNLHVEMEESLEKEGKTYQIHYVKEMAKELVRNYLVEARWLKEGYMPTLEEYMSVSMVTGTYGLMIARSYVGRGDIVTEDTFKWVSSYPPIIKASCVIVRLMDDIVSHKEEQERGHVASSIECYSKESGASEEEACEYISRKVEDAWKVINRESLRPTAVPFPLLMPAINLARMCEVLYSVNDGFTHAEGDMKSYMKSFFVHPMVV

SEQ ID NO: 7 - protein, 576 aa, Malus domestica
  REGION 1..576: note = Alpha-farnesene synthase
MEFRVHLQADNEQKIFQNQMKPEPEASYLINQRRSANYKPNIWKNDFLDQSLISKYDGDEYRKLSEKLIEEVKIYISAETMDLVAKLELIDSVRKLGLANLFEKEIKEALDSIAAIESDNLGTRDDLYGTALHFKILRQHGYKVSQDIFGRFMDEKGTLENHHFAHLKGMLELFEASNLGFEGEDILDEAKASLTLALRDSGHICYPDSNLSRDVVHSLELPSHRRVQWFDVKWQINAYEKDICRVNATLLELAKLNFNVVQAQLQKNLREASRWWANLGFADNLKFARDRLVECFSCAVGVAFEPEHSSFRICLTKVINLVLIIDDVYDIYGSEEELKHFTNAVDRWDSRETEQLPECMKMCFQVLYNTTCEIAREIEEENGWNQVLPQLTKVWADFCKALLVEAEWYNKSHIPTLEEYLRNGCISSSVSVLLVHSFFSITHEGTKEMADFLHKNEDLLYNISLIVRLNNDLGTSAAEQERGDSPSSIVCYMREVNASEETARKNIKGMIDNAWKKVNGKCFTTNQVPFLSSFMNNATNMARVAHSLYKDGDGFGDQEKGPRTHILSLLFQPLVN

SEQ ID NO: 8 - protein, 550 aa, Zingiber officinale
  REGION 1..550: note = Zingiber officinale - (S)-beta-bisabolene synthase
MELVDTPSLEVFEDVVVDRQVAGFDPSFWGDYFITNQKSQSEAWMNERAEELKNEVRSMFQNVTGILQTMNLIDTIQLLGLDYHFMEEIAKALDHLKDVDMSKYGLYEVALHFRLLRQKGFNISSDVFKKYKDKEGKFMEELKDDAKGLLSLYNAAYFGTKEETILDEAISFTKDNLTSLLKDLNPPFAKLVSLTLKTPIQRSMKRIFTRSYISIYQDEPTLNETILELAKLDFNMLQCLHQKELKKICAWWNNLNLDIMHLNFIRDRVVECYCWSMVIRHEPSCSRARLISTKLLMLITVLDDTYDSYSTLEESRLLTDAIQRWNPNEVDQLPEYLRDFFLKMLNIFQEFENELAPEEKFRILYLKEEWKIQSQSYFKECQWRDDNYVPKLEEHMRLSIISVGFVLFYCGFLSGMEEAVATKDAFEWFASFPKIIEACATIIRITNDITSMEREQKRAHVASTVDCYMKEYGTSKDVACEKLLGFVEDAWKTINEELLTETGLSREVIELSFHSAQTTEFVYKHVDAFTEPNTTMKENIFSLLVHPIPI

SEQ ID NO: 9 - protein, 553 aa, Cinnamomum camphora
  REGION 1..553: note = Plant terpene synthases
MDSMEVRRSAIYHSTFWDIDSIRALLARRDCTAAAALSPDHHKRLKERIQRRLQDITQPHHLLGLIDAVQRLGVAYQFEEEISDALHGLHSENTEHAIKDSLHHTSLYFRLLRQHGCNLSSDIFNKFKKEGGGFKASLCEDAMGLLSLYEAVRLSVKGEAILEEAQVFSIANLKILMERVERKLADRIEHALEIPLYWRAPRLEARWYIDVYEKEDGRIDDLLDFAKLDFNRVQMLYQTELKELSMWWELLGLPAKMGFFRDRLLENHLFSIAVVVEPQYSQCRVAITKAIVLMTAMDDFYDVHGLPDELKVFTDTVNRWDLEGIDQLPEYMKLYYLALYNTTNETAYIILKEKGFNATHYLKKLWAMQSNAYFREAQWFNSGYIPKFDEYLDNALVSVGAPFVLGLSYPMIQQQISKEEIDLIPEDLNLLRWASIIFRLYDDLATSKAEQQRGDVPKSIQCYMHETGSSEEVAANHIRDLISDAWKEVNAECLKPTSLSKHYVGVAPNSARSGVLMYHHDFDGFASPHGRTNAHITSIFFEPVPLKESINLG

SEQ ID NO: 10 - protein, 297 aa, synthetic construct
  REGION 1..297: note = alkane hydroxylase system with point mutation of F93S
MSTVTPKDIQFRKLMDAPLLAWPTMLLFFGSLGTLGLVDYHAVAGNIPLWVGTIINGLVIYLLFSCVHDASHGAVSRIKWINELPGHIGMFFSGPIAPFNVARFIHMQHHRFTNDSTMDPDHFGHKMDWLFPLRWSNFDYYYTKYFFMRAPAAMRKKFLPRLIIHFSVIIGVLIALTLAGYGLEALMLWILPTRISSFLFVMAFVYLPHEPFQSTAKEDEYRASNIRPGAEWLLTPLLAYQNYHLMHHLYPTAPFYNMIKLWELRHAEHMANSPLIVKTFGLQPLDEHARHAGRDAA

SEQ ID NO: 11 - protein, 297 aa, synthetic construct
  REGION 1..297: note = alkane hydroxylase system with point mutation of W189S
MSTVTPKDIQFRKLMDAPLLAWPTMLLFFGSLGTLGLVDYHAVAGNIPLWVGTIINGLVIYLLFSCVHDASHGAVSRIKWINELPGHIGMFFFGPIAPFNVARFIHMQHHRFTNDSTMDPDHFGHKMDWLFPLRWSNFDYYYTKYFFMRAPAAMRKKFLPRLIIHFSVIIGVLIALTLAGYGLEALMLSILPTRISSFLFVMAFVYLPHEPFQSTAKEDEYRASNIRPGAEWLLTPLLAYQNYHLMHHLYPTAPFYNMIKLWELRHAEHMANSPLIVKTFGLQPLDEHARHAGRDAA

SEQ ID NO: 12 - DNA, 2657 bp, synthetic construct
  source 1..2657: mol_type = other DNA
  misc_feature 1..2657: note = alkane hydroxylase system
aagcttgagtgtgcatgatattatcgcacggcaatgtgtaacgccgcaaagaggaaatgaatgttctcatctatgtgacacaaccggaatataacataactcaaattatacaaatatgtttagttgtcccttcacatttggcaatcccaaaatggcgaagtccttagcggtaatcccagcgtcgttgttctgggaccactcgaagaagatgagcccaaaaaatccgcttgcgcccggggccgtctgctcctagaaaccgcttcaaccggcgagccagcaggaatttcacacagaatgacaggagggacatatgtcgaccgtgaccccgaaggacatccagttccgcaagctcatggacgccccgctcctcgcctggccgaccatgctcctcttcttcggctcgctcggcaccctcggcctcgtggactaccacgccgtggccggcaacatcccgctctgggtgggcaccatcatcaacggcctcgtgatctacctcctcttctcgtgcgtgcacgacgcctcgcacggcgccgtgtcgcgcatcaagtggatcaacgagctcccgggccacatcggcatgttcttcttcggcccgatcgccccgttcaacgtggcccgcttcatccacatgcagcaccaccgcttcaccaacgactcgactatggacccggaccacttcggccacaagatggactggctcttcccgctccgctggtcgaacttcgactactactacaccaagtacttcttcatgcgcgccccggccgccatgcgcaagaagttcctcccgcgcctcatcatccacttctcggtgatcatcggcgtgctcatcgccctcaccctcgccggctacggccttgaggccctcatgctctggattctcccgacccgcatctcgtcgttcctcttcgtgatggccttcgtgtacctcccgcacgagccgttccagtcgaccgccaaggaggacgagtaccgcgcctcgaacatccgcccgggcgccgagtggctcctcaccccgctcctcgcctaccagaactaccacctcatgcaccacctctacccgaccgccccgttctacaacatgatcaagctctgggagctccgccacgccgagcacatggccaactcgccgctcatcgtgaagaccttcggcctccagccgctcgacgagcacgcccgccacgccggccgcgacgccgcctaaggaggtgctgccatgaagcgctggcagtgccgctactgctcgttcatctacgacgaggccctcggcctcccggccgagggcatcgccccgggcaccccgctctcggacgtgccggacgactggatgtgcccggactgcggcgccaccaaggtggacttcgacgagatctaaggaggtaactcaatgtctgaaaacgctccgctggttatcatcggtaccggtctggctggttacaacctggctcgtgaatggcgtaaactggacgctcagtctccgctgctgctgatcaccgctgacgacggtcgttcttactctaaaccgatgctgtctaccggtttctctaaaaacaaagacgctgacggtctggctatggctgaaccgggtgctatggctgaacagctgaaagctcgtatcctgacccacacccgtgttaccggtatcgacccgggtcacaaacgtatctggatcggtgaagaacaggttgaataccgtgacctggttctggcttggggtgctgaaaccatccgtgttccggttcagggtgacgctctggacgctatcttcccgatcaacgacctggaagactacgctcgtttccgtgctgctgctgctggtaaacgtcgtgttctgctgctgggtgctggtctgatcggttgcgagttcgctaacgacctgtctgctggtggttaccaggttgacgttgttgctccgtgcgaacaggttatgccgggtctgctgcacccggctgctgctgctgctgttcaggctggtctggaaggtctgggtgttcgtttccacctgggtccggttctgaaccgtctggaacgtgctggtgaatctctggaagctcacctgtctgacggtcaggctatcccgtgcgatctcgttgtaagcgctgttggtctgcgtccgcgtatcgacctggctgctgaagctggtctggaagttaaccgtggtgttgttgttaaccgtgaactgcgtacctctcacgctaacatctacgctctgggtgactgcgctgaagttgacggtctgaacctgctgtacgttatgccgctgatgtcttgcgctcgtgctctggctcagaccctggcgggtaaaccaaccgctgtaagctacggtccgatgccggttaccgttaaaaccccggtttgcccgctggttgtttctccgccgccgcgtggttctgacggtcagtggctggttgaaggttctggtgctgacctgaaagttctgtgccgtgacaccgctggtcgtctgctgggttacgctctgaccggtgctgctgtttctgaaaaactgtctctgaacaaagaactgccggctctgctggcttaatactagagtcggtcagtttcacctgatttacgtaaaaacccgcttcggcgggtttttgcttttggaggggcagaaagatgaatgactgtctactagagatcacgagaccggatcc

SEQ ID NO: 13 - skipped

SEQ ID NO: 14 - DNA, 2141 bp, Malus domestica
  source 1..2141: mol_type = unassigned DNA; note = Malus x domestica
  misc_feature 1..2141: note = Alpha-farnesene synthase
gaattcgattatgtggtgagggaattaccctgtattagtgcaacggcgaggaacgaaactggcgcggacatgattttgattgataattgaatgttgcggcttctacaattttcacaattatgtgagtattgtaagcaatcatgacatgataaattctgataacgtgtcgggtcatgtattcagccagtatgaggagcgccttcagaaatttatttgctttgtgagcggataacaattattagattcaccggcgagccagcaggaatttcactctagatgacaggagggacatatggagttccgcgtgcacctccaggccgacaacgagcagaagatcttccagaaccagatgaagccggagccggaggcctcgtacctcatcaaccagcgccgctcggccaactacaagccgaacatctggaagaacgacttcctcgaccagtcgctcatctcgaagtacgacggcgacgagtaccgcaagctctcggagaagctcatcgaggaggtgaagatctacatctcggccgagacgatggacctcgtggccaagctcgagctcatcgactcggtgcgcaagctcggcctcgccaacctcttcgagaaggagatcaaggaggccctcgactcgatcgccgccatcgagtcggacaacctcggcacccgcgacgacctctacggcaccgccctccacttcaagatcctccgccagcacggctacaaggtgtcgcaggacatcttcggccgcttcatggacgagaagggcaccctcgagaaccaccacttcgcccacctcaagggcatgctcgagctcttcgaggcctcgaacctcggcttcgagggcgaggacatcctcgacgaggccaaggcctcgctcaccctcgccctccgcgactcgggccacatctgctacccggactcgaacctctcgcgcgacgtggtgcactcgctcgagctcccgtcgcaccgccgcgtgcagtggttcgacgtgaagtggcagatcaacgcctacgagaaggacatctgccgcgtgaacgccaccctcctcgagctcgccaagctcaacttcaacgtggtgcaggcccagctccagaagaacctccgcgaggcctcgcgctggtgggccaacctcggcttcgccgacaacctcaagttcgcccgcgaccgcctcgtggagtgcttctcgtgcgccgtgggcgtggccttcgagccggagcactcgtcgttccgcatctgcctcaccaaggtgatcaacctcgtgctcatcatcgacgacgtgtacgacatctacggctcggaggaggagctcaagcacttcaccaacgccgtggaccgctgggactcgcgcgagacggagcagctcccggagtgcatgaagatgtgcttccaggtgctctacaacaccacctgcgagatcgcccgcgagatcgaggaggagaacggctggaaccaggtgctcccgcagctcaccaaggtgtgggccgacttctgcaaggccctcctcgtggaggccgagtggtacaacaagtcgcacatcccgaccctcgaggagtacctccgcaacggctgcatctcgtcgtcggtgtcggtgctcctcgtgcactcgttcttctcgatcacccacgagggcaccaaggagatggccgacttcctccacaagaacgaggacctcctctacaacatctcgctcatcgtgcgcctcaacaacgacctcggcacctcggccgccgagcaggagcgcggcgactcgccgtcgtcgatcgtgtgctacatgcgcgaggtgaacgcctcggaggagacggcccgcaagaacatcaagggcatgatcgacaacgcctggaagaaggtgaacggcaagtgcttcaccaccaaccaggtgccgttcctctcgtcgttcatgaacaacgccaccaacatggcccgcgtggcccactcgctctacaaggacggcgacggcttcggcgaccaggagaagggcccgcgcacccacatcctctcgctcctcttccagccgctcgtgaactgatactagaggagttggtagctcttgatccggcaaacaaaccaccgttggtagcggtggtttttttgtttgcaagcagcagattacgcgcagaaaaaaaggtactagagagtgcaagctt

SEQ ID NO: 15 - DNA, 2012 bp, Zingiber officinale
  source 1..2012: mol_type = unassigned DNA
  misc_feature 1..2012: note = Zingiber officinale - (S)-beta-bisabolene synthase
gaattcgattatgtggtgagggaattaccctgtattagtgcaacggcgaggaacgaaactggcgcggacatgattttgattgataattgaatgttgcggcttctacaattttcacaattatgtgagtattgtaagcaatcatgacatgataaattctgataacgtgtcgggtcatgtattcagccagtatgaggagcgccttcagaaatttatttgctttgtgagcggataacaattattagattcaccggcgagccagcaggaatttcactctagatgacaggagggacatatggagctcgtggacacgccgtcgctcgaggtgttcgaggacgtggtggtggaccggcaggtcgcgggcttcgaccccagcttctggggcgactacttcatcaccaaccagaagtcgcagtcggaggcctggatgaacgagcgcgccgaggagctcaagaacgaggtgcgctcgatgttccagaacgtgaccggcatcctgcagacgatgaacctcatcgacacgatccagctgctcggcctggactaccacttcatggaggagatcgccaaggcgctggaccatctcaaggacgtggacatgtcgaagtacggcctgtatgaggtggccctccatttccgcctgctccgccagaagggcttcaacatctcgtcggacgtgttcaagaagtataaggacaaggaaggcaagttcatggaggagctcaaggacgacgcgaagggcctgctctcgctgtacaacgccgcgtatttcggcaccaaggaagagacgatcctggacgaggccatctcgttcaccaaggacaacctgacgtcgctgctcaaggacctcaacccgcccttcgcgaagctggtgtcgctgaccctcaagacgccgatccagcgctcgatgaagcgcatcttcacccgctcgtacatctcgatctatcaggacgagcccaccctcaacgagacgatcctggagctcgccaagctggacttcaacatgctgcagtgcctccaccagaaggagctcaagaagatctgcgcgtggtggaacaacctgaacctcgacatcatgcatctgaacttcatccgcgaccgcgtggtggagtgctattgctggtcgatggtgatccgccatgagccctcgtgctcgcgcgcccgcctcatctcgaccaagctgctcatgctgatcaccgtgctcgacgacacgtacgactcgtattcgacgctcgaggagtcgcgcctgctcaccgacgccatccagcgctggaacccgaacgaggtggaccagctgcccgagtacctgcgcgacttcttcctgaagatgctcaacatcttccaggagttcgagaacgagctggccccggaggagaagttccgcatcctgtatctcaaggaagagtggaagatccagtcgcagtcgtacttcaaggagtgccagtggcgcgacgacaactatgtgcccaagctggaggagcatatgcgcctctcgatcatctcggtgggcttcgtgctgttctactgcggcttcctctcgggcatggaggaagccgtggcgaccaaggacgccttcgagtggttcgcgtcgttccccaagatcatcgaggcctgcgcgacgatcatccgcatcaccaacgacatcacgtcgatggagcgcgagcagaagcgcgcccatgtggcgtcgaccgtggactgctacatgaaggagtatggcacgtcgaaggacgtggcctgcgagaagctgctcggcttcgtggaggacgcgtggaagaccatcaacgaggagctgctcacggagacgggcctctcgcgcgaggtgatcgagctgtcgttccactcggcccagaccacggagttcgtgtataagcatgtggacgcgttcacggagccgaacaccaccatgaaggagaacatcttcagcctgctcgtccaccccatccccatctgacggtggtttttttgtttgcaagcagcagattacgcgcagaaaaaaaggtactagagagtgcaagctt

SEQ ID NO: 16 - DNA, 2021 bp, Cinnamomum camphora
  source 1..2021: mol_type = unassigned DNA
  misc_feature 1..2021: note = Plant terpene synthase
gaattcgattatgtggtgagggaattaccctgtattagtgcaacggcgaggaacgaaactggcgcggacatgattttgattgataattgaatgttgcggcttctacaattttcacaattatgtgagtattgtaagcaatcatgacatgataaattctgataacgtgtcgggtcatgtattcagccagtatgaggagcgccttcagaaatttatttgctttgtgagcggataacaattattagattcaccggcgagccagcaggaatttcactctagatgacaggagggacatatggactcgatggaggtgcgccgctcggccatctaccactcgaccttctgggacatcgactcgatccgcgccctcctcgcccgccgcgactgcaccgccgccgccgccctctcgccggaccaccacaagcgcctcaaggagcgcatccagcgccgcctccaggacatcacccagccgcaccacctcctcggcctcatcgacgccgtgcagcgcctcggcgtggcctaccagttcgaggaggagatctcggacgccctccacggcctccactcggagaacaccgagcacgccatcaaggactcgctccaccacacctcgctctacttccgcctcctccgccagcacggctgcaacctctcgtcggacatcttcaacaagttcaagaaggagggcggcggcttcaaggcctcgctctgcgaggacgccatgggcctcctctcgctctacgaggccgtgcgcctctcggtgaagggcgaggccatcctcgaggaggcccaggtgttctcgatcgccaacctcaagatcctcatggagcgcgtggagcgcaagctcgccgaccgcatcgagcacgccctcgagatcccgctctactggcgcgccccgcgcctcgaggcccgctggtacatcgacgtgtacgagaaggaggacggccgcatcgacgacctcctcgacttcgccaagctcgacttcaaccgcgtgcagatgctctaccagaccgagctcaaggagctctcgatgtggtgggagctcctcggcctcccggccaagatgggcttcttccgcgaccgcctcctcgagaaccacctcttctcgatcgccgtggtggtggagccgcagtactcgcagtgccgcgtggccatcaccaaggccatcgtgctcatgaccgccatggacgacttctacgacgtgcacggcctcccggacgagctcaaggtgttcaccgacaccgtgaaccgctgggacctcgagggcatcgaccagctcccggagtacatgaagctctactacctcgccctctacaacaccaccaacgagaccgcctacatcatcctcaaggagaagggcttcaacgccacccactacctcaagaagctctgggccatgcagtcgaacgcctacttccgcgaggcccagtggttcaactcgggctacatcccgaagttcgacgagtacctcgacaacgccctcgtgtcggtgggcgccccgttcgtgctcggcctctcgtacccgatgatccagcagcagatctcgaaggaggagatcgacctcatcccggaggacctcaacctcctccgctgggcctcgatcatcttccgcctctacgacgacctcgccacctcgaaggccgagcagcagcgcggcgacgtgccgaagtcgatccagtgctacatgcacgagaccggctcgtcggaggaggtggccgccaaccacatccgcgacctcatctcggacgcctggaaggaggtgaacgccgagtgcctcaagccgacctcgctctcgaagcactacgtgggcgtggccccgaactcggcccgctcgggcgtgctcatgtaccaccacgacttcgacggcttcgcctcgccgcacggccgcaccaacgcccacatcacctcgatcttcttcgagccggtgccgctcaaggagtcgatcaacctcggctgacggtggtttttttgtttgcaagcagcagattacgcgcagaaaaaaaggtactagagagtgcaagctt

SEQ ID NO: 17 - DNA, 2657 bp, synthetic construct
  source 1..2657: mol_type = other DNA
  misc_feature 1..2657: note = alkane hydroxylase system with point mutation of F93S
aagcttgagtgtgcatgatattatcgcacggcaatgtgtaacgccgcaaagaggaaatgaatgttctcatctatgtgacacaaccggaatataacataactcaaattatacaaatatgtttagttgtcccttcacatttggcaatcccaaaatggcgaagtccttagcggtaatcccagcgtcgttgttctgggaccactcgaagaagatgagcccaaaaaatccgcttgcgcccggggccgtctgctcctagaaaccgcttcaaccggcgagccagcaggaatttcacacagaatgacaggagggacatatgtcgaccgtgaccccgaaggacatccagttccgcaagctcatggacgccccgctcctcgcctggccgaccatgctcctcttcttcggctcgctcggcaccctcggcctcgtggactaccacgccgtggccggcaacatcccgctctgggtgggcaccatcatcaacggcctcgtgatctacctcctcttctcgtgcgtgcacgacgcctcgcacggcgccgtgtcgcgcatcaagtggatcaacgagctcccgggccacatcggcatgttcttctccggcccgatcgccccgttcaacgtggcccgcttcatccacatgcagcaccaccgcttcaccaacgactcgactatggacccggaccacttcggccacaagatggactggctcttcccgctccgctggtcgaacttcgactactactacaccaagtacttcttcatgcgcgccccggccgccatgcgcaagaagttcctcccgcgcctcatcatccacttctcggtgatcatcggcgtgctcatcgccctcaccctcgccggctacggccttgaggccctcatgctctggattctcccgacccgcatctcgtcgttcctcttcgtgatggccttcgtgtacctcccgcacgagccgttccagtcgaccgccaaggaggacgagtaccgcgcctcgaacatccgcccgggcgccgagtggctcctcaccccgctcctcgcctaccagaactaccacctcatgcaccacctctacccgaccgccccgttctacaacatgatcaagctctgggagctccgccacgccgagcacatggccaactcgccgctcatcgtgaagaccttcggcctccagccgctcgacgagcacgcccgccacgccggccgcgacgccgcctaaggaggtgctgccatgaagcgctggcagtgccgctactgctcgttcatctacgacgaggccctcggcctcccggccgagggcatcgccccgggcaccccgctctcggacgtgccggacgactggatgtgcccggactgcggcgccaccaaggtggacttcgacgagatctaaggaggtaactcaatgtctgaaaacgctccgctggttatcatcggtaccggtctggctggttacaacctggctcgtgaatggcgtaaactggacgctcagtctccgctgctgctgatcaccgctgacgacggtcgttcttactctaaaccgatgctgtctaccggtttctctaaaaacaaagacgctgacggtctggctatggctgaaccgggtgctatggctgaacagctgaaagctcgtatcctgacccacacccgtgttaccggtatcgacccgggtcacaaacgtatctggatcggtgaagaacaggttgaataccgtgacctggttctggcttggggtgctgaaaccatccgtgttccggttcagggtgacgctctggacgctatcttcccgatcaacgacctggaagactacgctcgtttccgtgctgctgctgctggtaaacgtcgtgttctgctgctgggtgctggtctgatcggttgcgagttcgctaacgacctgtctgctggtggttaccaggttgacgttgttgctccgtgcgaacaggttatgccgggtctgctgcacccggctgctgctgctgctgttcaggctggtctggaaggtctgggtgttcgtttccacctgggtccggttctgaaccgtctggaacgtgctggtgaatctctggaagctcacctgtctgacggtcaggctatcccgtgcgatctcgttgtaagcgctgttggtctgcgtccgcgtatcgacctggctgctgaagctggtctggaagttaaccgtggtgttgttgttaaccgtgaactgcgtacctctcacgctaacatctacgctctgggtgactgcgctgaagttgacggtctgaacctgctgtacgttatgccgctgatgtcttgcgctcgtgctctggctcagaccctggcgggtaaaccaaccgctgtaagctacggtccgatgccggttaccgttaaaaccccggtttgcccgctggttgtttctccgccgccgcgtggttctgacggtcagtggctggttgaaggttctggtgctgacctgaaagttctgtgccgtgacaccgctggtcgtctgctgggttacgctctgaccggtgctgctgtttctgaaaaactgtctctgaacaaagaactgccggctctgctggcttaatactagagtcggtcagtttcacctgatttacgtaaaaacccgcttcggcgggtttttgcttttggaggggcagaaagatgaatgactgtctactagagatcacgagaccggatcc

SEQ ID NO: 18 - DNA, 2657 bp, synthetic construct
  source 1..2657: mol_type = other DNA
  misc_feature 1..2657: note = alkane hydroxylase system with point mutation of W189S
aagcttgagtgtgcatgatattatcgcacggcaatgtgtaacgccgcaaagaggaaatgaatgttctcatctatgtgacacaaccggaatataacataactcaaattatacaaatatgtttagttgtcccttcacatttggcaatcccaaaatggcgaagtccttagcggtaatcccagcgtcgttgttctgggaccactcgaagaagatgagcccaaaaaatccgcttgcgcccggggccgtctgctcctagaaaccgcttcaaccggcgagccagcaggaatttcacacagaatgacaggagggacatatgtcgaccgtgaccccgaaggacatccagttccgcaagctcatggacgccccgctcctcgcctggccgaccatgctcctcttcttcggctcgctcggcaccctcggcctcgtggactaccacgccgtggccggcaacatcccgctctgggtgggcaccatcatcaacggcctcgtgatctacctcctcttctcgtgcgtgcacgacgcctcgcacggcgccgtgtcgcgcatcaagtggatcaacgagctcccgggccacatcggcatgttcttcttcggcccgatcgccccgttcaacgtggcccgcttcatccacatgcagcaccaccgcttcaccaacgactcgactatggacccggaccacttcggccacaagatggactggctcttcccgctccgctggtcgaacttcgactactactacaccaagtacttcttcatgcgcgccccggccgccatgcgcaagaagttcctcccgcgcctcatcatccacttctcggtgatcatcggcgtgctcatcgccctcaccctcgccggctacggccttgaggccctcatgctctcgattctcccgacccgcatctcgtcgttcctcttcgtgatggccttcgtgtacctcccgcacgagccgttccagtcgaccgccaaggaggacgagtaccgcgcctcgaacatccgcccgggcgccgagtggctcctcaccccgctcctcgcctaccagaactaccacctcatgcaccacctctacccgaccgccccgttctacaacatgatcaagctctgggagctccgccacgccgagcacatggccaactcgccgctcatcgtgaagaccttcggcctccagccgctcgacgagcacgcccgccacgccggccgcgacgccgcctaaggaggtgctgccatgaagcgctggcagtgccgctactgctcgttcatctacgacgaggccctcggcctcccggccgagggcatcgccccgggcaccccgctctcggacgtgccggacgactggatgtgcccggactgcggcgccaccaaggtggacttcgacgagatctaaggaggtaactcaatgtctgaaaacgctccgctggttatcatcggtaccggtctggctggttacaacctggctcgtgaatggcgtaaactggacgctcagtctccgctgctgctgatcaccgctgacgacggtcgttcttactctaaaccgatgctgtctaccggtttctctaaaaacaaagacgctgacggtctggctatggctgaaccgggtgctatggctgaacagctgaaagctcgtatcctgacccacacccgtgttaccggtatcgacccgggtcacaaacgtatctggatcggtgaagaacaggttgaataccgtgacctggttctggcttggggtgctgaaaccatccgtgttccggttcagggtgacgctctggacgctatcttcccgatcaacgacctggaagactacgctcgtttccgtgctgctgctgctggtaaacgtcgtgttctgctgctgggtgctggtctgatcggttgcgagttcgctaacgacctgtctgctggtggttaccaggttgacgttgttgctccgtgcgaacaggttatgccgggtctgctgcacccggctgctgctgctgctgttcaggctggtctggaaggtctgggtgttcgtttccacctgggtccggttctgaaccgtctggaacgtgctggtgaatctctggaagctcacctgtctgacggtcaggctatcccgtgcgatctcgttgtaagcgctgttggtctgcgtccgcgtatcgacctggctgctgaagctggtctggaagttaaccgtggtgttgttgttaaccgtgaactgcgtacctctcacgctaacatctacgctctgggtgactgcgctgaagttgacggtctgaacctgctgtacgttatgccgctgatgtcttgcgctcgtgctctggctcagaccctggcgggtaaaccaaccgctgtaagctacggtccgatgccggttaccgttaaaaccccggtttgcccgctggttgtttctccgccgccgcgtggttctgacggtcagtggctggttgaaggttctggtgctgacctgaaagttctgtgccgtgacaccgctggtcgtctgctgggttacgctctgaccggtgctgctgtttctgaaaaactgtctctgaacaaagaactgccggctctgctggcttaatactagagtcggtcagtttcacctgatttacgtaaaaacccgcttcggcgggtttttgcttttggaggggcagaaagatgaatgactgtctactagagatcacgagaccggatcc

SEQ ID NO: 19 - DNA, 1250 bp, synthetic construct
  source 1..1250: mol_type = other DNA
  misc_feature 1..1250: note = M1-AlkB-RR, lacking the reductase: the first part of the orf of M1-alkB, and of the rubredoxin
agcccaaaaaatccgcttgcgcccggggccgtctgctcctagaaaccgcttcaaccggcgagccagcaggaatttcacacagaatgacaggagggacatatgtcgaccgtgaccccgaaggacatccagttccgcaagctcatggacgccccgctcctcgcctggccgaccatgctcctcttcttcggctcgctcggcaccctcggcctcgtggactaccacgccgtggccggcaacatcccgctctgggtgggcaccatcatcaacggcctcgtgatctacctcctcttctcgtgcgtgcacgacgcctcgcacggcgccgtgtcgcgcatcaagtggatcaacgagctcccgggccacatcggcatgttcttcttcggcccgatcgccccgttcaacgtggcccgcttcatccacatgcagcaccaccgcttcaccaacgactcgactatggacccggaccacttcggccacaagatggactggctcttcccgctccgctggtcgaacttcgactactactacaccaagtacttcttcatgcgcgccccggccgccatgcgcaagaagttcctcccgcgcctcatcatccacttctcggtgatcatcggcgtgctcatcgccctcaccctcgccggctacggccttgaggccctcatgctctggattctcccgacccgcatctcgtcgttcctcttcgtgatggccttcgtgtacctcccgcacgagccgttccagtcgaccgccaaggaggacgagtaccgcgcctcgaacatccgcccgggcgccgagtggctcctcaccccgctcctcgcctaccagaactaccacctcatgcaccacctctacccgaccgccccgttctacaacatgatcaagctctgggagctccgccacgccgagcacatggccaactcgccgctcatcgtgaagaccttcggcctccagccgctcgacgagcacgcccgccacgccggccgcgacgccgcatgaagcgctggcagtgccgctactgctcgttcatctacgacgaggccctcggcctcccggccgagggcatcgccccgggcaccccgctctcggacgtgccggacgactggatgtgcccggactgcggcgccaccaaggtggacttcgacgagatctgatactagagtcggtcagtttcacctgatttacgtaaaaacccgcttcggcgggtttttgcttttggaggggcagaaagatgaatgactgtctactagagatca

SEQ ID NO: 20 - DNA, 894 bp, synthetic construct
  source 1..894: mol_type = other DNA
  misc_feature 1..894: note = alkane 1-mono- oxygenase AlkB
atgtcgaccgtgaccccgaaggacatccagttccgcaagctcatggacgccccgctcctcgcctggccgaccatgctcctcttcttcggctcgctcggcaccctcggcctcgtggactaccacgccgtggccggcaacatcccgctctgggtgggcaccatcatcaacggcctcgtgatctacctcctcttctcgtgcgtgcacgacgcctcgcacggcgccgtgtcgcgcatcaagtggatcaacgagctcccgggccacatcggcatgttcttcttcggcccgatcgccccgttcaacgtggcccgcttcatccacatgcagcaccaccgcttcaccaacgactcgactatggacccggaccacttcggccacaagatggactggctcttcccgctccgctggtcgaacttcgactactactacaccaagtacttcttcatgcgcgccccggccgccatgcgcaagaagttcctcccgcgcctcatcatccacttctcggtgatcatcggcgtgctcatcgccctcaccctcgccggctacggccttgaggccctcatgctctggattctcccgacccgcatctcgtcgttcctcttcgtgatggccttcgtgtacctcccgcacgagccgttccagtcgaccgccaaggaggacgagtaccgcgcctcgaacatccgcccgggcgccgagtggctcctcaccccgctcctcgcctaccagaactaccacctcatgcaccacctctacccgaccgccccgttctacaacatgatcaagctctgggagctccgccacgccgagcacatggccaactcgccgctcatcgtgaagaccttcggcctccagccgctcgacgagcacgcccgccacgccggccgcgacgccgcctaa

SEQ ID NO: 21 - DNA, 159 bp, synthetic construct
  source 1..159: mol_type = other DNA
  misc_feature 1..159: note = For rubredoxin peptide
atgaagcgctggcagtgccgctactgctcgttcatctacgacgaggccctcggcctcccggccgagggcatcgccccgggcaccccgctctcggacgtgccggacgactggatgtgcccggactgcggcgccaccaaggtggacttcgacgagatctaa

SEQ ID NO: 22 - DNA, 1159 bp, synthetic construct
  source 1..1159: mol_type = other DNA
  misc_feature 1..1159: note = Rubredoxin reductase DNA - Nucleic acid Seqeunce
atgtctgaaaacgctccgctggttatcatcggtaccggtctggctggttacaacctggctcgtgaatggcgtaaactggacgctcagtctccgctgctgctgatcaccgctgacgacggtcgttcttactctaaaccgatgctgtctaccggtttctctaaaaacaaagacgctgacggtctggctatggctgaaccgggtgctatggctgaacagctgaaagctcgtatcctgacccacacccgtgttaccggtatcgacccgggtcacaaacgtatctggatcggtgaagaacaggttgaataccgtgacctggttctggcttggggtgctgaaaccatccgtgttccggttcagggtgacgctctggacgctatcttcccgatcaacgacctggaagactacgctcgtttccgtgctgctgctgctggtaaacgtcgtgttctgctgctgggtgctggtctgatcggttgcgaattcgctaacgacctgtctgctggtggttaccaggttgacgttgttgctccgtgcgaacaggttatgccgggtctgctgcacccggctgctgctgctgctgttcaggctggtctggaaggtctgggtgttcgtttccacctgggtccggttctgaaccgtctggaacgtgctggtgaatctctggaagctcacctgtctgacggtcaggctatcccgtgcgatctcgttgtaagcgctgttggtctgcgtccgcgtatcgacctggctgctgaagctggtctggaagttaaccgtggtgttgttgttaaccgtgaactgcgtacctctcacgctaacatctacgctctgggtgactgcgctgaagttgacggtctgaacctgctgtacgttatgccgctgatgtcttgcgctcgtgctctggctcagaccctggcgggtaaaccaaccgctgtaagctacggtccgatgccggttaccgttaaaaccccggtttgcccgctggttgtttctccgccgccgcgtggttctgacggtcagtggctggttgaaggttctggtgctgacctgaaagttctgtgccgtgacaccgctggtcgtctgctgggttacgctctgaccggtgctgctgtttctgaaaaactgtctctgaacaaagaactgccggctctgctggcttaattaa

SEQ ID NO: 23 - protein, 301 aa, Acinetobacter guillouiae
  REGION 1..301: note = Desaturase domain-containing protein; Acinetobacter guillouiae
MRTMSKSKPFDHALRELTKAPLIALPTLFLFIICLAILAIVDYCVILGTIPLWVGMIINGFTIYFLFSPIHDASHGAFSQIKWINEIPGHIGMIFFGPIAPFNLARFIHMQHHRCTNEKENDPDYFGHKMDIFFPLRWSNFDYYYSKYFFLRASQAMRKRLLPHLVIHFSAIIAVLAALIYFGYGLEALMLWILPTRISSFLFVSAFVFLPHEPFQSTAKENEYRATNVRPGAEWLLTPLLAYQNYHLMHHLYPTAPFYRMLKLWELKHDQHVANDPLIIKTFGLAPIKNDQSKTTNDAST

SEQ ID NO: 24 - protein, 301 aa, Acinetobacter sp.
  source 1..301: note = Acinetobacter sp. MYb10
  REGION 1..301: note = Fatty acid desaturase; Acinetobacter sp. MYb10; Taxonomic NCBI Identifier 1827285
MRTINKSTPSDHALRELTKAPLIALPTLFLFIICLSILAIVDYCVILGMVPLWIGMIINGFTIYFLFSPIHDASHGAFSRIKWINEIPGHIGMIFFGPIAPFNLARFIHMQHHRCTNEKENDPDYFGHKMDIFFPLRWSNFDYFYSKYFFLRASQAMRKRLLPHLVIHFASIIGVLAALIYFGYGLEALMLWILPTRISSFLFVTAFVFLPHEPFQSTAKENEYRATNVRPGAEWLLTPLLTYQNYHLMHHLYPTAPFYRMLKLWKLKHEHHVANDPLIIKTFGLAPIKSDQTKMKNDVSS

SEQ ID NO: 25 - protein, 297 aa, synthetic construct
  REGION 1..297: note = Protein variant 1
MSTVTPKDIQYRKLMDAPLLAWPTMVLFFGSIGTLGLVDYHAVAGNIPLWVGTIINGLVIYLLFSCVHDASHGAVSRIRWINELPGHIGMFFSGPIAPFNVARFIHMQHHRWTNDTTMDPDHFGHKMDWLFPLMWSNYDFYFTKYFWMRAPAAMRKKFLPRLIIHFSVIIGVLIALTLAGYGLEALMLSILPTRISSFLFVMAFVYVPHEPFQSTAKEDEFRASNIRPAAEWLLTPLLAYQNYHLMHHLYPTAPFYNMIKLWELRHAEHMANSPLIVMTFGLQPLDEHARHAGRDAA

SEQ ID NO: 26 - protein, 297 aa, synthetic construct
  REGION 1..297: note = Protein variant 2
MSTVTPKDIQFRKLMDAPLLAYPTMLLFFGSLGTLGLVDYHAVAGNIPLWVGTIINGLVVFLLFSCVHDASHGAVSMVKWINELPGHIGMFFSGPIAPFNVARFVHMQHHRFTNDSTMDPDHFGHMMDWLFPLRWTNFDYWYTKYFFMRAPAAMRKKFLPRLVIHFSVVIGVLIALTLAGYGLEALMLSILPTRISSFVFVMAFVYLPHEPFNSTAKEDEYRASNIRPGAEWLITPLLAYQNYHLMHHLYPTAPFYNMIKLWELRHAEHMANSPLIVKTFGLQPLDEHAKHAGRDAA

SEQ ID NO: 27 - protein, 297 aa, synthetic construct
  REGION 1..297: note = Protein variant 3
MTTVTPKDIQFRKLMDGPLLAYPTRLLYFGSLGTLGLVDYHAVAGNIPIWVGSIINGLVVYILFSCVHDASHGGVSRIKWLNEVPGHIGMFFFAPIAPYQVARFIHRQHHRFTNDSTMDPDHFGHKMDWLFPLRWSNFDYWWTKYFFMRAPAAMRKRFLPRLIVHFSVIIGVLIALTLAGYGIEALMLWILPTMVSSWLFVMAFVYLPHEPFQSTAKEDEYRASNIRPGAEWLVTPLLAYQNYHLMHHLYPSAPFYNMIKLFEVRHAEHMANSPLIVMTYGIQPLDEHARHAGRDAA

SEQ ID NO: 28 - protein, 297 aa, synthetic construct
  REGION 1..297: note = Protein variant 4
MSTITPKDIQFRKLMDAPLLAWPTMLVFFGSLGTLGLVDYHAVAGNIPLWVGTIINGLVIYLLFSCVHDASHGAVSRIKFINELPGHIGMFYSGPIGPFNVARFIHRQHHRFTNDSTMDPDHYGHKMDWLWPLKWSNYEYYWSMYFFMRAPGAMRKKFLPRLIIHFTVIIGILIALTLAGYGIEALMLSILPTRVTSYLFVMAFVFLPHEPFQSTAKEDEYRASNIRPGAEWLITPLLAYQNYHLMHHLYPTAPYYNMIKLWELRHADHMGNSPLIVMTFGLNPVDEHARHAGRDAA

SEQ ID NO: 29 - protein, 297 aa, synthetic construct
  REGION 1..297: note = Protein variant 5
MTTVTPKDIQFRKLMDGPLLAFPTMIVFFGTLGTLGLLDYHAVAGNIPIFVGTIINGIIIYVLFSCVHDATHGAVSRIKWLNELPGHIAMFFSGPIAPFNVARYIHMQHHRFSNDSTKDPDHFGHMMDWLFPLRWSNFDYYYTKFFFMRAPAARRMKWVPRLIIHFTVIIGLLIAITLAGYGIDALMLWVLPTRISSYLFVMAFIYIPHEPFQSSGKEEEYRGSNIRPGAEWLLTPLIAYQNYHLMHHLYPTAPFFNMIKLWELRHAEHMANSPIILKTYGLQPLEEHAMHAGREAA

SEQ ID NO: 30 - protein, 297 aa, synthetic construct
  REGION 1..297: note = Protein variant 6
MSTVTPKEIQFRKLKEAPLLAYPTMLLFFGSIGSLGLIEYHAVAGNIPLYVGSIINGLVLYLIYTCVHDASHGAVSRIKYINELPGHIGMWFWGPIAPFNVARFVHMQHHRFTNDSTMDPDHFGHMMDWLYPIRFSQFDYYYSKYFFRKAPAAMRKKFIPRLIVHWTVLIGVLIAVTLAGYGLEALRLWILPTRISSFLFVMAWVYLPHEPWQTTAKEEEFRASNIRPGAEWLLTPLLAYQNYHLMHHLYPTAPFYNMIKLWEVRHADHMGNSPLIVKTFGIQPVDEHAMHGGRDAA

SEQ ID NO: 31 - protein, 297 aa, synthetic construct
  REGION 1..297: note = Protein variant 7
MTTVTPKDIQFRRLMDAPLVAYPTMLIFFGSLGTLGLVDWHALAGNLPVYVGTIINGVVIFLLYSCIHDASHAAVSRIKFINEVPGHIGMFFSGPIAPFNIARFVHKQHHMFTNDSTRDPDHWGHKMDWLFPLKWSNYEYFYTKYYFMRAPAARRKKFLPRLIIHFSVIIGVVIGISLAGYGLEALRLSILPSRISSFLFVMAFVYLPHEPFNSTAKEDEYKASNIRPGAEWLVSPLLAYQNYHLMHHLYPTAPFYNMIKLFELRHAEHKGNSPLIVKTFGLQPVDEHAKHAGRDAA

SEQ ID NO: 32 - protein, 297 aa, synthetic construct
  REGION 1..297: note = Protein variant 8
MSTVSPKDINFRRLMDGPLLGYPTMVIFFGTLGTLGLVEYHALAGNIPLWVASIINGLLLFLLFSCIHDASHGAVSRIKWINELPGHIGKFFFGPIAPFNVARFIHKQHHKFTNDSTKDPDHFGHKRDWLFPLRWSNWDFYWSRYYWMRAPAGRKKKFLPMLLIHFSVIIGVLIGLTIAGFAVDALMLSILPSMLSSFLFLMAYLYIPHEPFQSTAKEEEFRASNIKPGAEWLLSPLLGYQNYHLMHHLYPTAPWYQMIKLWELRHADHMAQSPVILKTFALQPIDEHARHAGRDAA

SEQ ID NO: 33 - protein, 297 aa, synthetic construct
  REGION 1..297: note = Protein variant 9
MTTVTPRDVQWRKLKDAPLLAYPTKVLFFASIGTLGLLDFHAVAGNLPLWVGSIINGLIIYILFSCVHDASHGAVSMVKWINELPGHIAMWFSGPIAPFNIARFIHRQHHRWTNDSTKDPDHFGHKMDFLFPLKWSNFDYFFTKYFWMRGPAAMRMKWLPRVLIHWSVIIALLIALTLAGWAVEALMLWLLPSMISSFLFVMGYVYLPHEPFQSTGKEDEYRASNIRPGGEWLITPLLAYNNYHIMHHLYPTAPYFNMIKLWELRHAEHKANSPIILRTFALQPLDEHAKHAGRDAG

SEQ ID NO: 34 - protein, 297 aa, synthetic construct
  REGION 1..297: note = Protein variant 10
MSTVTPKDIQWRMLKDAPLLAWPTMVLFFGSLGTLGLVDWHGVAGNLPLYVGTIINGLVIYIVFSCVHDASHGAVSRIKWINDLPGHIGMFFSGPIAPFQLARFIHMQHHRWTNETSKDPDHFGHKMEYLYPLMWSNFDYFYTKWWYKRGPAGMMKRFLPMVLIHFSVIVGLIIGLTIAGWGLEALMLSVLPTRLSSFLFVRAWVYLPHEPYQSTAKEDEYMASNLKPGAEYLLSPLLGYQNYHLMHHLYPTAPFFNMIKLWEVRHAEHMANSPLIVRTFGLNPIDEHAMHAGMDAG

SEQ ID NO: 35 - protein, 297 aa, synthetic construct
  REGION 1..297: note = Protein variant 11
MSTLTPKEVNYMKLREAPVVAFPTMIVFFGSLGTLGLVEYHALAGNIPLWVGTIINGVLIYVLYSCVHDATHGAVSMIKFINEVPGHIGMFFFGPIAPFNIARWIHRQHHRFTNDTTRDPDHFGHKKEFVFPLKWSNWDYWYTKYYYRRAPAAMRRKFLPMLIVHWSLVVGVILALTVAGWGLEGLRLWILPSRLSTWLFLKAYVFLPHDPYQSTAKEDEYRASNVRPGADWLITPLLGYQNYHVMHHLYPTAPFYNMIRLWEVRHAEHMGNSPLILMSFALQPVDEHARHAGRDGA

SEQ ID NO: 36 - protein, 297 aa, synthetic construct
  REGION 1..297: note = Protein variant 12
MSTLTPKDIQFKMLMEGPVIAWPSMVLWFGSLGSVALLDYHAVGANIPLYVGTIIQGVVIYLLFTCLHDASHGAVSRLKWINELPGHIGRYFSGPIGPYQVARFIHMQHHRWSNDSTMDPDHFAHKREYLWPIRFSNYDYYYSRYYWKRAPAAMRKKFVPMIVIHFSLVVGLLIALTLGGYALDALRLWILPTRVTSFIFVRGFVYLPHEPFQTTAKEDEYKASNVRPGAEFLLTPLLAYQNYHVMHHLYPSAPFYQMIMLWELRHAEHMANSPLIVMSFGIQPLDDHARHAAMEAA

SEQ ID NO: 37 - protein, 297 aa, synthetic construct
  REGION 1..297: note = Protein variant 13
MSTVTPMDLQFMRLMDGPIIGWPTMLLFFGSIGTLGLVDWHAVGGQVPIWVGTIINGLVIWLLFTCVHDASHGAVSRIRYVNEIPGHIGMFYSGPIAPFNLARYVHMQHHRFTNDSSMDPDHWGHMRDWLFPLRWSNWDWFWTKFWFMRAPAAMKKKYVPKLIIHFSLLLGIVIALTLAGYGIDALKLWILPSKITSFLFVKAFVFIPHDPFNTTAKEDDYRGSNIMPGADWLLTPLLGYQNYHLMHHLYPTAPYYNMIKLWEIRHGDHMANSPIILRTFGVQPVDEHGKHAAKEAA

SEQ ID NO: 38 - protein, 297 aa, synthetic construct
  REGION 1..297: note = Protein variant 14
MSTVTPRDIQFMKLMEGPLLAYPTMIVFFGSLGTIGLLEFHAVGGQIPIWVGTIIQGILIYLIWSCIHDATHGGVSMIMFINEIPGHIGMYFFAPIAPYNIARWIHRQHHRFTNDTTRDPDHFAHMRDWLWPVRWTNYDFYYTKYFFRRAPAGRRRKYLPRILIHYSIIVGVVIAITIAGFGVEALMLWLLPTKVSSFIFLKAYLFVPHEPWNSTAKEEDWRASNIRPGGEYLLTPLLAYQNYHVMHHLYPTAPYWNMIMLYELRHGEHRGNSPIIVRTWGINPLEDHAMHAGRDAA

SEQ ID NO: 39 - protein, 297 aa, synthetic construct
  REGION 1..297: note = Protein variant 15
MSTVSPRDVNFRKLKDAPLLGYPTRLIFFGSVGTLGLVDFHAIAANIPVWVGTIINGIVIFLLFSCVHDATHGAVSKIRYVNEVPGHIGRFFSGPIAPFNVARYVHKNHHRFTNDSTMDPDHWGHKMDFVYPLMYTNFDYYYSKFWFKKAPAAMRRKFLPRLIVHFTILVAVIIGISLGGFGVDGLRIWILPTRISSYIFVRGWLYVPHEPFQSTAREEEWKASNIRPAAEFLVTPLIAYQNYHLMHHLYPTAPFYQMIKLWEIRHADHKGQSPLIVKSWGLNPIDDHAKHGAKDGG

SEQ ID NO: 40 - protein, 297 aa, synthetic construct
  REGION 1..297: note = Protein variant 16
MSTVTPKEINFMRLMDAPILAWPSMVVWFGSLGTIGLVDYHGLAGNIPIYVGSIIQGLLIFILFSCVHDATHGGVSMVKWINEIPGHIGRFFFGPIAPYNVARFLHMNHHKYTNESTRDPDHFAHRRDWIWPLMWSQWDYWYTRWFYMKGPAARRRKYLPRLIIHFSIILAIVIAVTVAGWALDGLMVSVLPTMITSFLFVKAYLWVPHEPFQSSAMEEEFRASNIRPGADWLVSPLIAYQNYHIMHHLYPTAPWFNMIKLWEVRHADHMANSPLIVRTFALNPVEEHGKHAGMDAA

SEQ ID NO: 41 - protein, 297 aa, synthetic construct
  REGION 1..297: note = Protein variant 17
MSTITPKEIQFKRLREAPLIAWPSMVIFFGTIGTIGLLEWHALGGNVPLYVATIIQGIVIYVLWSCLHDASHAAVSMVMFVNELPGHIGMFFSGPIGPFNLARWLHMNHHRFSNESTMDPDHWGHKMEWLFPVRYTNWDFWYTKYFWRMAPGAMMMKYLPRILIHFTVVIGIIVAVTIAGWGLEGLMLSLLPTMLSTWVFLKAYIWLPHEPFNTSAMEDEYMASNVKPGAEWLLSPLLAYQNYHIMHHLYPTAPFFQMIKLWELRHAEHKGQSPVILMTWGLQPIEDHAMHAGRDGA

SEQ ID NO: 42 - protein, 297 aa, synthetic construct
  REGION 1..297: note = Protein variant 18
MTTLTPREIQYRMLMEAPLIGWPSMIVWFASVGTLGLIEYHGVAAQIPIFVATIIQGLILYILYTCVHDASHGAVSMIKWLNDLPGHIGMFFSGPIGPYNVARYLHMNHHRWTNETSMDPDHWGHKKDYIWPVMWSNFDFWYSMFWWMMAPAGMRKKYIPRVIIHWSVVIAIILGLSLAGWAIEALMLSLLPTRISTFVFVRAFLYLPHEPYQTSARDEDYRASNLMPAAEYLLTPLIAYQNYHVMHHLYPSAPFYNMIKLWEVRHADHMANSPIIVMTFAINPIEEHGMHAGRDAA

SEQ ID NO: 43 - protein, 296 aa, Pseudomonas sp.
  source 1..296: note = Pseudomonas sp. CMR5c
  REGION 1..296: note = AZC18544.1: protein C4K40_3156
MSTGTRDSTQFRKLMDAPLLAWPTVLLFFGSLSILGLVDYLALAGTIPLWTGTIINGLVIYLLFSCVHDASHGAVSRIKWINELPGHVGMFFFGPIAPFNVARFIHMQHHRFTNDNALDPDHFGHKMDWLFPLRWSNFDYYYTKYFFMRAPAAMRRKFLPRLIIHFSVIIAVLLALTLAGYGLEALMLWILPTRISSFLFVMAFVYLPHEPFQSTAKEDEYRASNIRPGAEWLLTPLLAYQNYHLMHHLYPTAPFYNMIKLWELRQAEHMAKHPLIVNTFGLRPVSQSSRARGDAA

SEQ ID NO: 44 - DNA, 1046 bp, synthetic construct
  source 1..1046: mol_type = other DNA
  misc_feature 1..1046: note = synthetic gene
atgtcgaccggcacccgcgactcgacccagttccgcaagctcatggacgccccgctcctcgcctggccgaccgtgctcctcttcttcggctcgctctcgatcctcggcctcgtggactacctcgccctcgccggcaccatcccgctctggaccggcaccatcatcaacggcctcgtgatctacctcctcttctcgtgcgtgcacgacgcctcgcacggcgccgtgtcgcgcatcaagtggatcaacgagctcccgggccacgtgggcatgttcttcttcggcccgatcgccccgttcaacgtggcccgcttcatccacatgcagcaccaccgcttcaccaacgacaacgccctcgacccggaccacttcggccacaagatggactggctcttcccgctccgctggtcgaacttcgactactactacaccaagtacttcttcatgcgcgccccggccgccatgcgccgcaagttcctcccgcgcctcatcatccacttctcggtgatcatcgccgtgctcctcgccctcaccctcgccggctacggcctcgaggccctcatgctctggatcttaccgacccgcatctcgtcgttcctcttcgtgatggccttcgtgtacctcccgcacgagccgttccagtcgaccgccaaggaggacgagtaccgcgcctcgaacatccgcccgggcgccgagtggctcctcaccccgctcctcgcctaccagaactaccacctcatgcaccacctctacccgaccgccccgttctacaacatgatcaagctctgggagctccgccaggccgagcacatggccaagcacccgctcatcgtgaacaccttcggcctccgcccggtgtcgcagtcgtcgcgcgcccgcggcgacgccgcatgaagcgctggcagtgccgctactgctcgttcatctacgacgaggccgccggcctcccggccgacggcatcgccccgggcaccccgctctcggacgtgccggacgactggatgtgcccggactgcggcgccaccaaggtggacttcgacgagatctga

SEQ ID NO: 45 - protein, 52 aa, Pseudomonas sp.
MKRWQCRYCSFIYDEAAGLPADGIAPGTPLSDVPDDWMCPDCGATKVDFDEI

(19) United States
(12) Patent Application Publication
(10) Pub. No.: US 2024/0287478 A1
(43) Pub. Date: Aug. 29, 2024

(54) ARTIFICIAL ALKANE OXIDATION SYSTEM FOR ALLYLIC OXIDATION OF A TERPENE SUBSTRATE

(71) Applicant: ISOBIONICS B.V., Geleen (NL)

(72) Inventors: Martinus Julius BEEKWILDER, Geleen (NL); Matthew Quinn STYLES, Geleen (NL); Susan Eline BOUWMEESTER, Den Hoorn (NL); Hendrik Jan BOSCH, Wageningen (NL)

(21) Appl. No.: 18/681,152

(22) PCT Filed: Aug. 6, 2022
(86) PCT No.: PCT/EP2022/072171

§ 371 Date: Feb. 5, 2024

(30) Foreign Application Priority Data
Aug. 6, 2021 (EP) 21190191.3

(51) Int. Cl. C12N 9/02; C07K 14/195; C12N 9/88; C12N 15/52; C12P 5/00

(52) U.S. Cl. CPC C12N 9/0095; C07K 14/195; C12N 9/0077; C12N 9/88; C12N 15/52; C12P 5/007

(57) ABSTRACT

An artificial alkane oxidation system comprising components: a. an oxidase enzyme; and b. one or more enzymes to provide one or more alkanes. The artificial alkane oxidation system optionally has c. an electron transfer compound suitable to transfer at least one electron to the oxidase enzyme; and further optionally has d. an electron transfer compound regeneration enzyme suitable to reduce the electron transfer compound of c. when it is in its oxidized state. The oxidase enzyme is an amino acid sequence with a sequence identity of at least 70% with any one of SEQ ID NO: 1, 10, 11, 23 to 43 or a fragment thereof or a variant thereof.

### FIELD OF INVENTION

The present invention relates to an artificial alkane oxidation system, the synthetic nucleic acid sequences, the expression cassettes for the same, a method of oxidation of at least one alkane, preferably alkene, more preferably terpene, the oxidised terpene product produced thereof and use of the artificial alkane oxidation system.

### BACKGROUND OF THE INVENTION

Terpenes are widely used in food processing, personal care and care chemical products. Terpenes are extracted from their natural sources like oils from citrus fruits, tree barks, etc. Terpenes are also produced by chemical as well as biosynthetic pathways.

A number of citrus oils, including orange oil and mandarin oil, generally contain terpenes, more specifically sesquiterpene aldehydes, called alpha sinensal and beta sinensal. Sinensals are part of the aldehyde fraction of orange oil and is considered a quality parameter, in particular for the organoleptic properties. Beta sinensal odour is described as orange-sweet-fresh-waxy-juicy, while alpha sinensal odour has been described as citrus-orange-mandarin. Sinensals are used as flavouring agents for citrus products.

Chemical synthesis routes for the aldehydes fraction of the oil are known in the art. Synthesis of alpha sinensal is described by Büchi (1974; JACS 96, 7573-4), starting from (E)-3-methyl-2,4-pentadien-1-ol, using pyridine and phosphorus tribromide. Synthesis of beta sinensal is described among others by Bertele & Schudel (U.S. Pat. Nos. 3,699,169; 3,974,226), starting from beta farnesene using ozonolysis, and by Hiyama (1978, Tetrahedron letters 19, 3051-4) starting from a triene aldehyde.

However, chemically synthesised compounds or compounds prepared by synthetic routes are categorised separately from naturally produced aldehyde fractions. Biosynthetically obtained compounds are generally labelled natural and have a price advantage. However so far not all terpene compounds have been disclosed to be amenable to biosynthetic production.

For example, biosynthesis of sinensal is not known in the art. Structurally, alpha and beta sinensal are related to the sesquiterpene hydrocarbons alpha and beta farnesene. Both sinensals carry an allylic aldehyde group, compared to the farnesenes. Alpha and beta farnesene are both well-known components of Citrus oils. However, a biosynthetic pathway in which a sinensal is made by an allylic oxidation of a farnesene by an oxidative enzyme such as a monooxygenase towards an allylic alcohol, and further oxidized by an alcohol dehydrogenase towards sinensal is not known in the art. Also, no monooxygenase or any other oxidative enzyme that performs the proper allylic oxidation on farnesene has been described in the art.

Arfmann (Biocatalys, 1988, Vol. 2, pp. 59-67) proposed that sinensal can be produced from nerolidol. Microorganism incubated with trans-nerolidol gave the 12-hydroxy-trans-nerolidol that was further oxidized to the 12-carboxylic acid of trans-nerolidol. It is disclosed to use microorganism for allylic oxidation (or “omega oxidation”) of different stereo isomers of nerolidol, towards 12-hydroxy nerolidol (Hrdlicka, Biotechnol. Prog. 2004, 20, 368-376). However, no further conversion of 12-hydroxy nerolidol is disclosed in the art.

Abraham (Z. Naturforsch. 47c, 851-858, 1992) discloses a sulfonated derivative of farnesene oxidized on its di-allylic group by Nocardia sp. DSM 43130 and by Pseudomonas lapsa DSM 50274. However, activity of the strains used on non-sulfonated forms of farnesene is not disclosed in the art.

Iurescia Sandra et al. (Applied and Environmental Microbiology, American Society For Microbiology, US, vol. 65, no. 7, 1 Jul. 1999 (1999-07-01), pages 2871-2876) discloses isolated as well as bio-transformed Pseudomonas M1 and 4 open reading frames (ORF), named myrA (an aldehyde dehydrogenase), myrB (an alcohol dehydrogenase), myrC (an acyl-coenzyme A (CoA) synthetase), and myrD (an enoyl-CoA hydratase). Iurescia et al proposed a pathway for 13-myrcene catabolism in Pseudomonas sp. strain M1 involving these four proteins. According to Iurescia and co-workers, these four enzymes start their catabolic work with a myrcene alcohol. However, the enzyme catalysing the first step of converting myrcene to myrcene alcohol is not disclosed by Iurescia and co-workers at the time.

In another research project the authors reported the result of a shotgun sequencing project of Pseudomonas strain M1 (Iurescia et al 1999, Soares-Castro & Santos 2013). The authors disclosed over 6000 tentative genes in Pseudomonas sp. M1. Amongst the 6000 tentative genes is one coding for a protein with the UniProt database identifier W5IZV3 which is designated as a fatty acid desaturase. It is identical to the protein of SEQ ID NO: 1 of the present invention. It is to be noted that the database entry is flagged with the warning that the sequence is preliminary data. Furthermore, this fatty acid desaturase is not linked to the other work of the authors, reported in Applied And Environmental Microbiology, 1 Jul. 1999 pages 2871-2876, as the four genes myrA to myrD identified in said first study on myrcene catabolism are UniProt database entries Q9XD59, Q9XD58, Q9XD57 and Q9XD60, respectively. Moreover, these four myrcene catabolism related sequences share less than 35% sequence identity with SEQ ID NO: 1 of the present invention.

Allylic oxidation of other terpenes such as amorphadiene (Ro et al 2006, Nature 440: 940-943), germacrene A (https://doi.org/10.1104/pp. 19.00629) and santalene (Diaz-Chavez PLOS One. 2013 Sep. 18; 8(9):e75053.) is disclosed to be mediated by plant cytochrome P450 enzymes. However, none of the enzymes are disclosed to mediate allylic oxidation of farnesene towards sinensal.

DEGENHARDT J ET AL, PHYTOCHEMISTRY, ELSEVIER, AMSTERDAM, NL, vol. 70, no. 15-16, 1 Oct. 2009 (20 Sep. 10, 2001), pages 1621-1637, provides a review of the monoterpene and sesquiterpene synthases.

EP 2 706 111 A 1 discloses pathways and mechanisms to confer production of carbon-based products of interest such as ethanol, ethylene, chemicals, polymers, n-alkanes, isoprenoids, pharmaceutical products or intermediates thereof in photoautotrophic organisms such that these organisms efficiently convert carbon dioxide and light into carbon-based products of interest, and in particular the use of such organisms for the commercial production of ethanol, ethylene, chemicals, polymers, n-alkanes, isoprenoids, pharmaceutical products or intermediates thereof.

In Williams Shoshana C Et Al, Journal Of Inorganic Biochemistry, Elsevier Inc, US, vol. 219, 16 Mar. 2021 (2021-03-16), ISSN: 0162-0134 the authors report a rubredoxin-fused alkane monooxygenase gene from Dietzia cinnamea capable of oxidizing long-chain fully saturated alkanes like heptane, octane, nonane, decane, undecane, tridecane in cell lysates. The author provides a relationship analysis on the class of rubredoxin-fused alkane monooxygenase and the role of this class of alkane monooxygenase in cell wall biosynthesis. Further, a point mutation introduced in the full length protein from Dietzia cinnamea resulted in less epoxide products than the wildtype produced with the fully saturated alkanes heptane, octane, nonane, decane, undecane, tridecane as substrates. The mutant produced aldehyde products and epoxide products in about equal amounts.

It was an object of the present invention to provide for a process of complete biosynthesis of terpene-based aldehyde and/or alcohol products. A further object of the present invention was to provide a fermentative production system for terpene-based aldehyde and/or alcohol products for example the first fermentation based production of sinensal.

### SUMMARY OF THE INVENTION

Surprisingly, it has been found that the above object is met by providing an artificial alkane oxidation system, the synthetic nucleic acid sequences, the expression cassettes for the same, a method of oxidation of at least one alkane, preferably alkene, more preferably terpene, the oxidised terpene product produced thereof and use of the artificial alkane oxidation system.

Accordingly, in one aspect, the present invention is directed to an artificial alkane oxidation system comprising:
a. an oxidase enzyme with an amino acid sequence with a sequence identity of at least 62%, 66%, 69%, 70%, 75%, 80%, 85%, 90%, 94%, 95%, 98% or 99% with % with any one of SEQ ID NO: 1, 10, 11, 23 to 43 or a fragment thereof, and
b. one or more enzymes providing at least one alkane, preferably a terpene synthase protein, optionally with
C. an electron transfer compound suitable to transfer at least one electron to the oxidase enzyme and further optionally
d. an electron transfer compound regeneration enzyme suitable to reduce the electron transfer compound of c. when it is in its oxidized state;
   wherein the oxidase enzyme and the one or more enzymes providing at least one alkane are not found in combination in nature.

The artificial alkane oxidation system is kept under conditions suitable for the production of at least one alkane and/or is brought into contact with at least one alkane under conditions suitable to oxidise the said at least one alkane. In the later case, in one embodiment, the alkane oxidation system can be component a. plus c. and optionally d., but lack b.

In another aspect, the presently claimed invention is directed to synthetic nucleic acid sequence with a sequence identity of at least 62%, 66%, 69%, 70%, 75%, 80%, 85%, 90%, 94%, 95%, 98% or 99% with any one of SEQ ID NO:4, SEQ ID NO:12, SEQ ID NO:17, SEQ ID NO:18, SEQ ID NO: 19, or SEQ ID NO: 44, wherein the synthetic nucleic acid sequence comprises a nucleic acid sequence encoding the aforementioned oxidase enzyme, and optionally the rubredoxin peptide and/or the rubredoxin reductase peptide.

In another aspect, the presently claimed invention is directed to a kit comprising
a first isolated nucleic acid sequence encoding for a protein with a sequence identity of at least 62%, 66%, 69%, 70%, 75%, 80%, 85%, 90%, 94%, 95%, 98% or 99% with any one of SEQ ID NO: 1, 10, 11, 23 to 43 wherein the first nucleic acid sequence comprises a nucleic acid sequence encoding the oxidase enzyme and
a second isolated nucleic acid sequence, not physically connected to the first nucleic acid encoding an electron transfer protein and
optionally a third nucleic acid sequence encoding an electron transfer protein reductase, wherein the third nucleic acid sequence may be fused to the first and/or second nucleic acid sequence.

In another aspect, the presently claimed invention is directed to an expression cassette comprising:
the synthetic nucleic acid sequence; or
the nucleic acid sequence encoding the alkane oxidation system; or
the nucleic acid sequence(s) of the alkane oxidation system including:
a. a nucleic acid sequence for an oxidase enzyme:
having a sequence identity of at least 70% with SEQ ID NO: 20, or
encoding for an oxidase enzyme with a sequence identity of at least 70% with any one of SEQ ID NO: 1, 10, 11, 23 to 43; and
b. a nucleic acid sequence for a rubredoxin peptide:
having a sequence identity of at least 70% with SEQ ID NO: 21, or
encoding for a rubredoxin of SEQ ID NO: 2 or SEQ ID NO: 45, and optionally
c. a nucleic acid sequence for a rubredoxin reductase peptide:
having a sequence identity of at least 70% with of SEQ ID NO: 22, or
encoding for a rubredoxin reductase peptide of SEQ ID NO: 3.

In another aspect, the presently claimed invention is directed to an isolated expression cassette comprising the nucleic acid sequence of the alkane oxidation system, the nucleic acid sequences comprising:
a. a nucleic acid sequence for an oxidase enzyme with a sequence identity of at least 62%, 66%, 69%, 70%, 75%, 80%, 85%, 90%, 94%, 95%, 98% or 99% with SEQ ID NO:20, or encoding for an oxidase enzyme with a sequence identity of at least 62%, 66%, 69%, 70%, 75%, 80%, 85%, 90%, 94%, 95%, 98% or 99% with any one of SEQ ID NO: 1, 10, 11, 23 to 43 and
b. a nucleic acid sequence for a rubredoxin peptide with a sequence identity of at least 62%, 66%, 69%, 70%, 75%, 80%, 85%, 90%, 94%, 95%, 98% or 99% with SEQ ID NO:21, or encoding a rubredoxin with at least 62%, 66%, 69%, 70%, 75%, 80%, 85%, 90%, 94%, 95%, 98% or 99% sequence identity to SEQ ID NO: 2 and optionally
c. a nucleic acid sequence for a rubredoxin reductase peptide with a sequence identity of at least 62%, 66%, 69%, 70%, 75%, 80%, 85%, 90%, 94%, 95%, 98% or 99% with of SEQ ID NO:22 or combination of b and c with a.

In another aspect, the presently claimed invention is directed to a method of oxidation of at least one alkane substrate, preferably alkene substrate, more preferably terpene substrate, the method comprising:
a. providing under conditions suitable for the oxidation of the terpene substrate(s)
   i. the artificial alkane oxidase system disclosed above, and/or
   ii. expression of the synthetic nucleic acid sequence disclosed above, or expression cassette disclosed above and/or
   iii. expression of a nucleic acid sequence encoding the oxidase enzyme with an amino acid sequence with a sequence identity of at least 62%, 66%, 69%, 70%, 75%, 80%, 85%, 90%, 94%, 95%, 98% or 99% with any one of SEQ ID NO: 1, SEQ ID NO: 10, SEQ ID NO: 11, SEQ ID NO: 23 to 43 or a fragment thereof or a variant thereof, and
   iv. optionally the at least one alkane, preferably alkene, more preferably terpene substrate if not produced by the alkane oxidation system;
   for oxidation of the at least one alkane, preferably alkene, more preferably terpene substrate,
b. allylic oxidation of the alkane, preferably alkene, more preferably terpene substrate to produce at least one oxidised alkane, preferably alkene, more preferably terpene product
c. optionally extracting the at least one oxidised alkane, preferably alkene, more preferably terpene product.

In a further aspect, the presently claimed invention is directed to a method of production of at least one oxidised alkane product, preferably alkene product, more preferably terpene product as defined herein, the method comprising:
a. providing under conditions suitable for the oxidation of the alkane, preferably alkene, more preferably terpene substrate(s)
   i. the artificial alkane oxidase system disclosed above, and/or
   ii. expression of the synthetic nucleic acid sequence disclosed above, or expression cassette disclosed above and/or
   iii. expression of a nucleic acid sequence encoding the oxidase enzyme with an amino acid sequence with a sequence identity of at least 62%, 66%, 69%, 70%, 75%, 80%, 85%, 90%, 94%, 95%, 98% or 99% with any one of SEQ ID NO: 1, 10, 11, 23 to 43 or a fragment thereof or a variant thereof, and
   iv. optionally the at least one alkane, preferably alkene, more preferably terpene substrate if not produced by the alkane oxidation system;
   for oxidation of the at least one alkane, preferably alkene, more preferably terpene substrate,
b. allylic oxidation of the alkane, preferably alkene, more preferably terpene substrate to produce at least one oxidised terpene product
c. optionally extracting the at least one oxidised alkane, preferably alkene, more preferably terpene product.

If the alkane oxidase system of the invention is producing the one or more alkane, preferably alkene, more preferably terpene substrates or it is present in conjunction with a system producing the one or more substrates or is included in a host cell that produces at least one substrate then no addition of substrate is required. Nonetheless one or more substrates may still be added even under such circumstances, for example to make more substrate(s) available or provide desired substrates that are not present before to the alkane oxidation system and in the methods of the invention.

In another aspect, the presently claimed invention is directed to use of the alkane oxidation system disclosed above, or the non-human host cells of the invention, the fermentation compositions as disclosed herein, or expression of nucleic acid sequence disclosed above, or expression cassette disclosed above for oxidation of the terpene substrate and hence production of one or more oxidised terpene products.

In another aspect, the presently claimed invention is directed to a non-human host cell comprising the artificial alkane oxidation system, the synthetic nucleic acid, the expression cassettes or production of the alkane oxidised product by the methods as described.

In another aspect, the presently claimed invention is directed to a composition produced by the method as described above, or by the non-human host cell, wherein the composition comprises myrcene aldehyde, alpha sinensal, beta sinensal, trans alpha santalol and trans beta santalol, lanceol-aldehyde, nootkatone, vetivone, rotundone, rebaudiosides, 8-hydroxygeraniol, 8-hydroxynerol, 9,10-epoxygeranylacetone, hexadecenal, farnesol, denderalasin and bicyclo-octanediol, oxidized alpha guaiene, oxidized beta guaiene, or combination thereof, and optionally one or more terpene substrates and optionally the oxidase enzyme as described.

In another aspect, the presently claimed invention is directed to a fermentation composition comprising:

the non-human host cell cultured in a culture medium; and
the at least one oxidised terpene product produced from the non-human host cell as a major compound,
wherein the fermentation composition includes the terpene substrate and optionally one or more co-products are minor compounds.

In an embodiment, the fermentation composition is an intermediate product that is subjected to further processing that include but is not limited to extraction, concentration, purification, drying, heat treatment, pressure treatment, vacuum treatment, or combination thereof.

In another embodiment, the fermentation composition is final product that is not subjected to further processing or is subjected to further processing.

In another aspect, the presently claimed invention is directed to a method of fermentative production of at least one oxidised terpene product, the method comprising:

providing a non-human host cell with the oxidase enzyme system;
culturing the non-human host cell in the culture medium to produce at least one oxidised terpene product.

In another aspect, the present invention is directed to a method for preparing a variant polypeptide having the oxidase enzyme activity, the method comprising steps of:
a) selecting a synthetic nucleic acid as described herein (with a sequence identity of at least 70% with any one of SEQ ID NO: 4, 12, 17, 18, 19, or 44, wherein the synthetic nucleic acid sequence comprises a nucleic acid sequence encoding the oxidase enzyme, and optionally the electron transfer protein and/or the electron transfer protein reductase as defined herein) or a nucleic acid encoding an amino acid sequence with a sequence identity of at least 62%, 66%, 69%, 70%, 75%, 80%, 85%, 90%, 94%, 95%, 98% or 99% of Any one of SEQ ID NO: 1, 10, 11, 23 to 43 or a fragment thereof;
b) modifying the selected nucleic acid to obtain at least one mutant nucleic acid;
c) transforming the non-human host cells with the mutant nucleic acid sequence to express the oxidase enzyme encoded by the mutant nucleic acid sequence;
d) screening the oxidase enzyme for at least one modified property in addition to the functionality of the oxidase enzyme encoded by the selected nucleic acid of a); and,
e) optionally, repeating the process steps (a) to (d) to obtain an oxidase enzyme with a desired variant functionality;
f) optionally, isolating the mutant nucleic acid sequence from the transformed non-human host cell obtained in step (c).

### DETAILED DESCRIPTION OF THE DRAWINGS

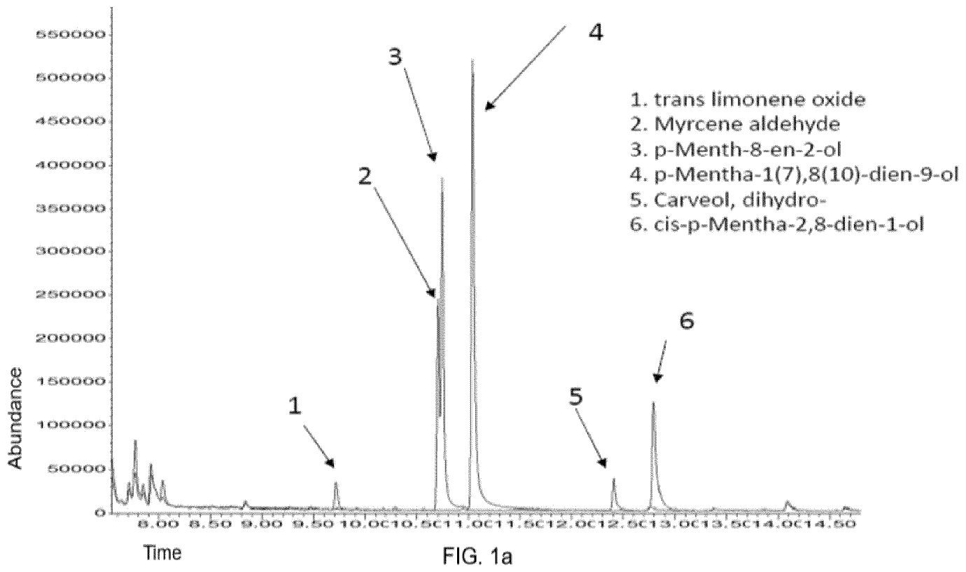

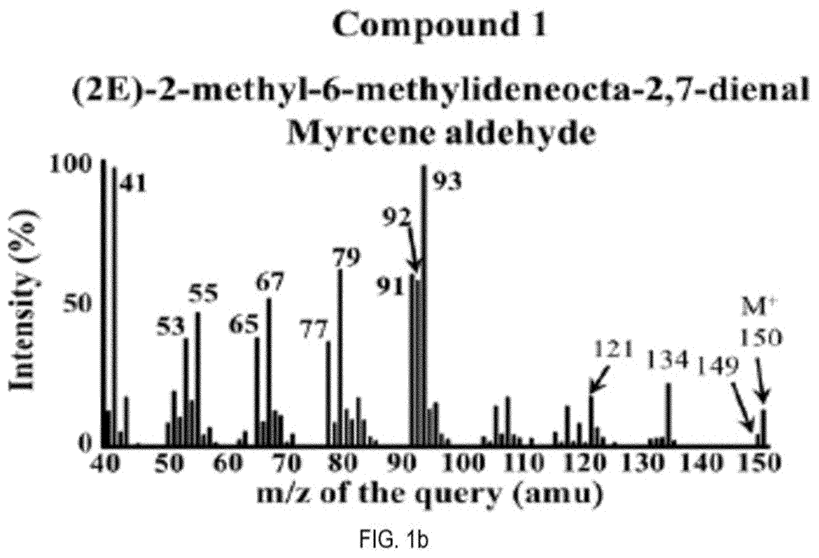

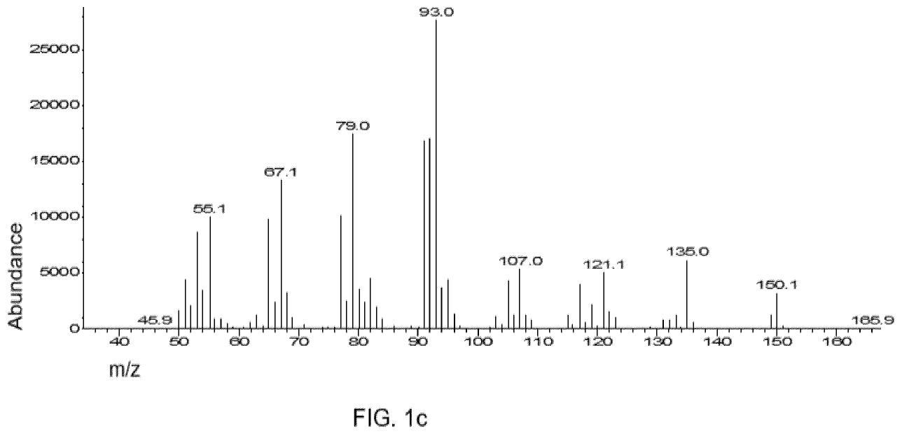

FIG. 1a illustrates an embodiment with Myrcene conversion by E. coli BL21 with pET-DUET as control and pET-M1-alkB-rr-rrr carrying an alkane oxidation system.

FIG. 1b is a copy for demonstration purposes of a mass spectrum as found in: Soares-Castro P, Montenegro-Silva P, Heipieper H J, Santos P M. 2017. Functional characterization of a 28-kilobase catabolic island from Pseudomonas sp. strain M1 involved in biotransformation of β-myrcene and related plant-derived volatiles. Appl Environ Microbiol 83:e03112-16. Figure S2.

FIG. 1c illustrates the mass spectra identified as myrcene aldehyde from Myrcene conversion shown in FIG. 1a after comparison with the Literature reference in FIG. 1b.

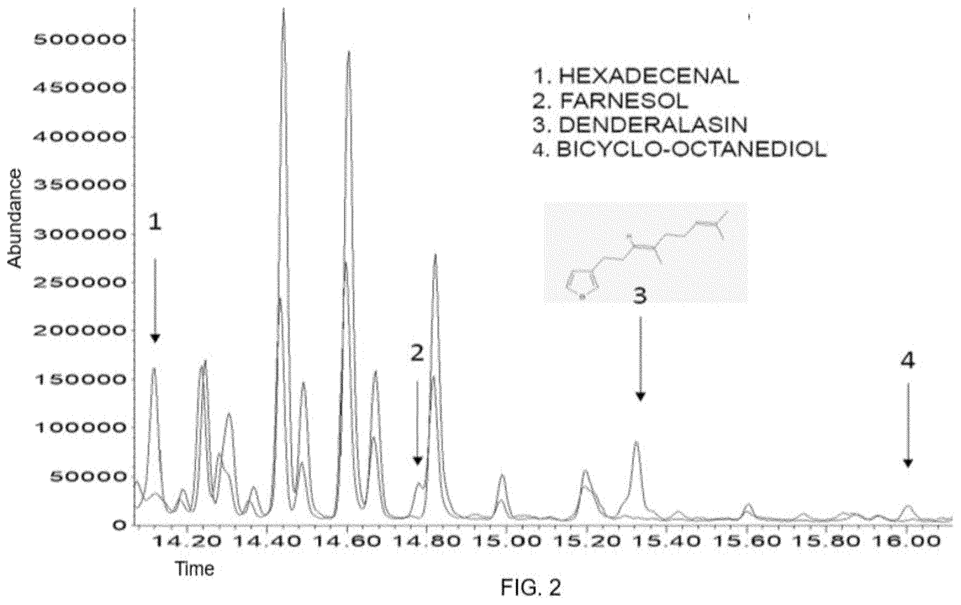

FIG. 2 illustrates an embodiment with Beta Farnesene conversion by E. coli BL21 with pET-DUET as control and pET-M1-alkB-rr-rrr carrying an alkane oxidation system.

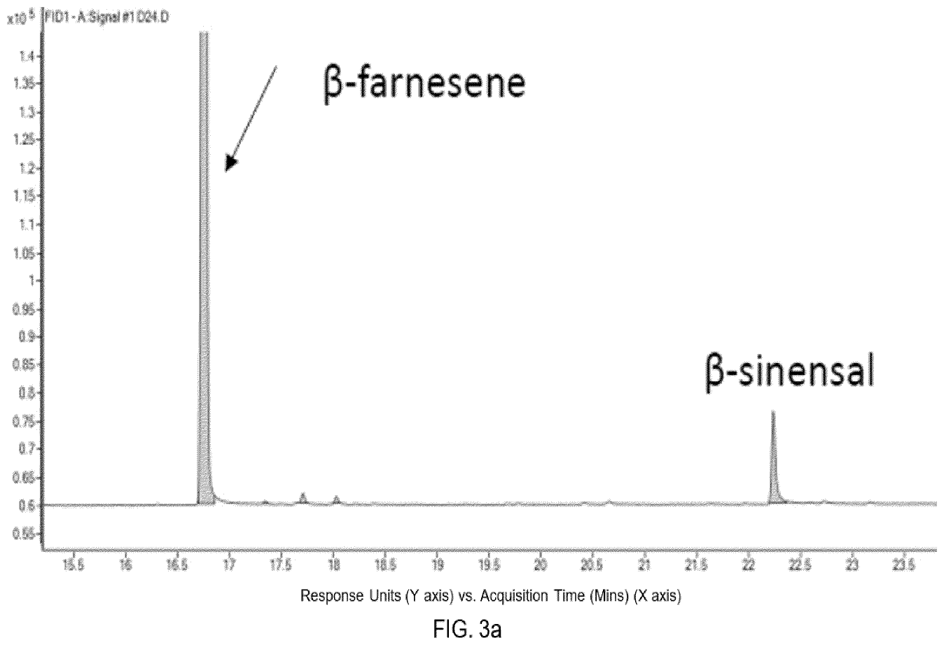

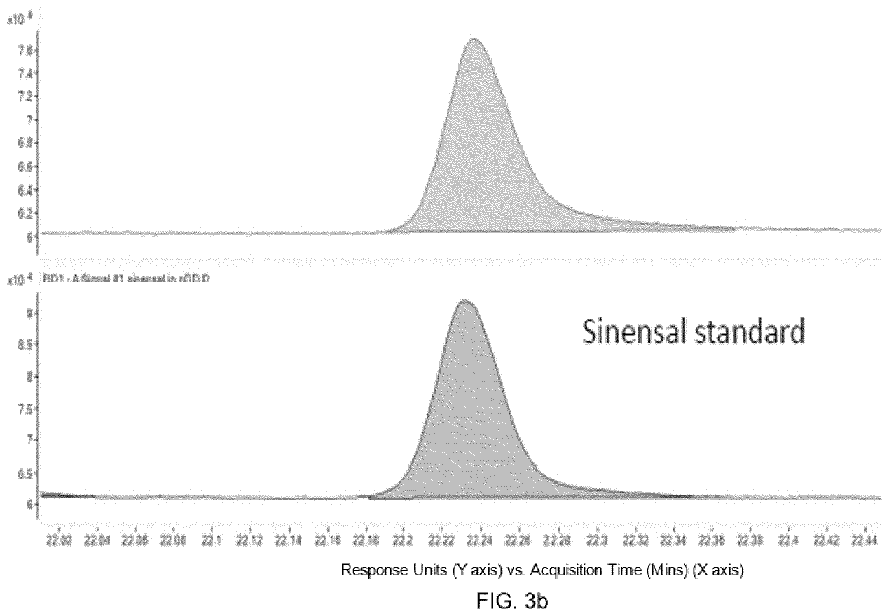

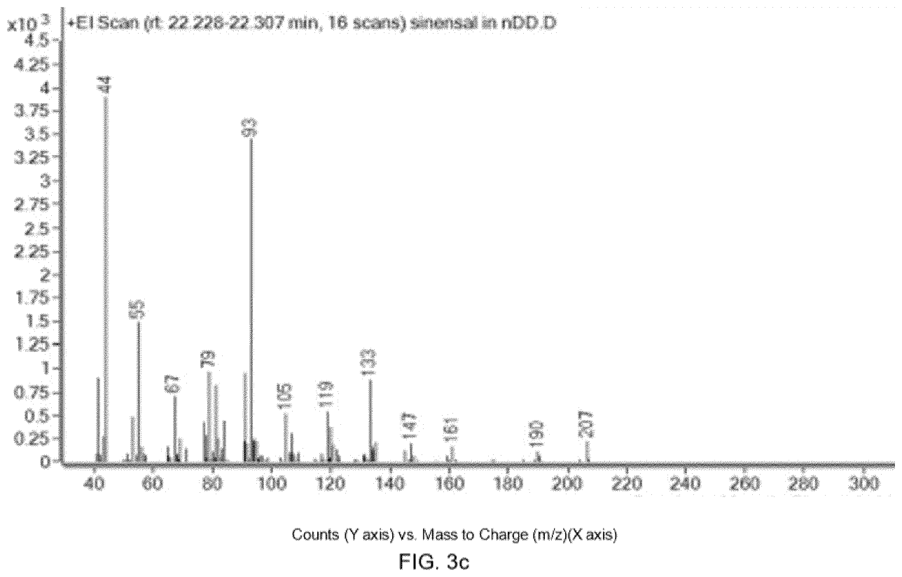

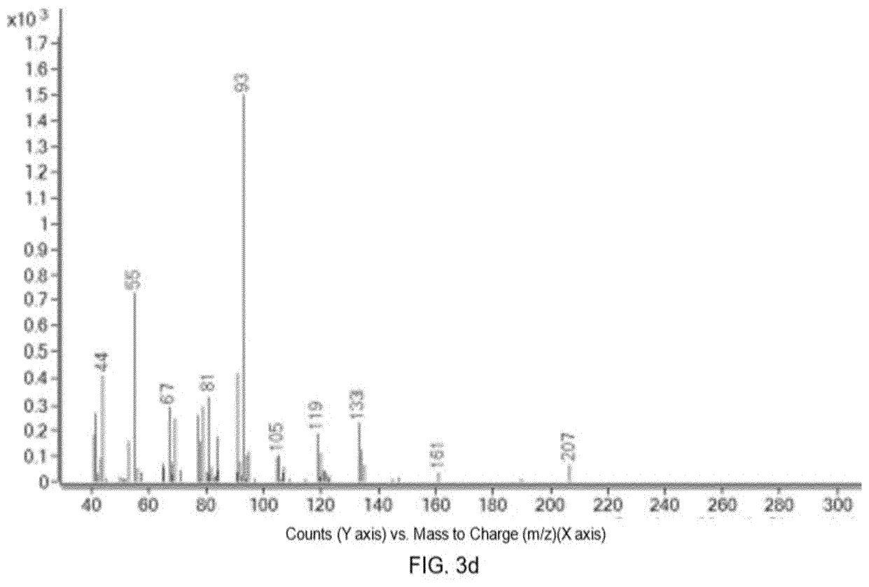

FIG. 3a illustrates an embodiment with Sinensal produced by Rhodobacter strain Rs265-9c-pBBR-MVA-AaBFS-M1-alkB-rr-rrr carrying an alkane oxidation system.

FIG. 3b illustrates an embodiment with Sinensal produced by Rhodobacter strain Rs265-9c-pBBR-MVA-AaBFS-M1-alkB-rr-rrr confirmed by comparison with Sinensal standard.

FIG. 3c. illustrates Mass spectra of Sinensal produced with Rs265-9c-pBBR-MVA-AaBFS-M1-alkB-rr-rrr.

FIG. 3d. Illustrates Mass spectra of a sinensal standard.

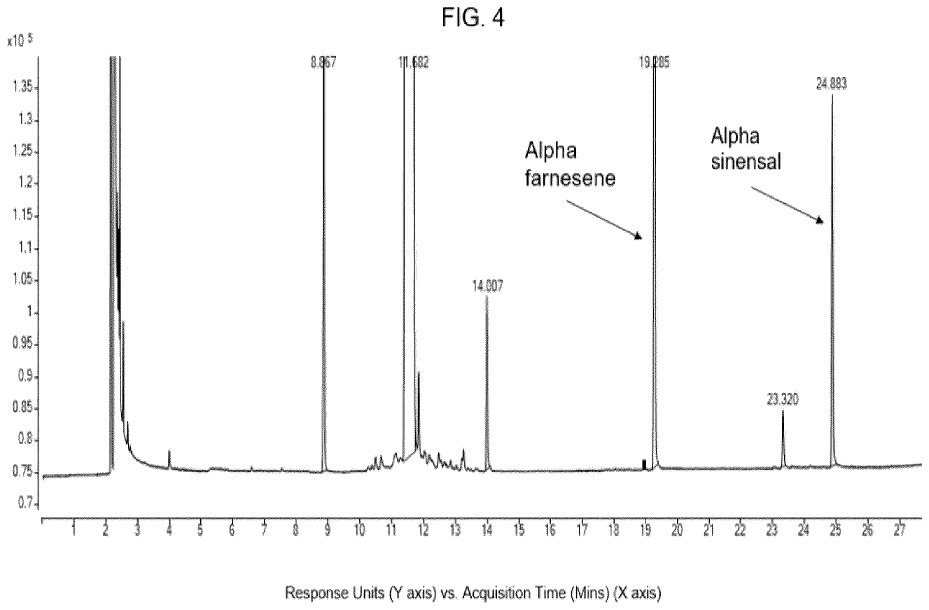

FIG. 4 illustrates Mass spectra demonstrating Alpha sinensal production using an alkane oxidation system. See example section for details.

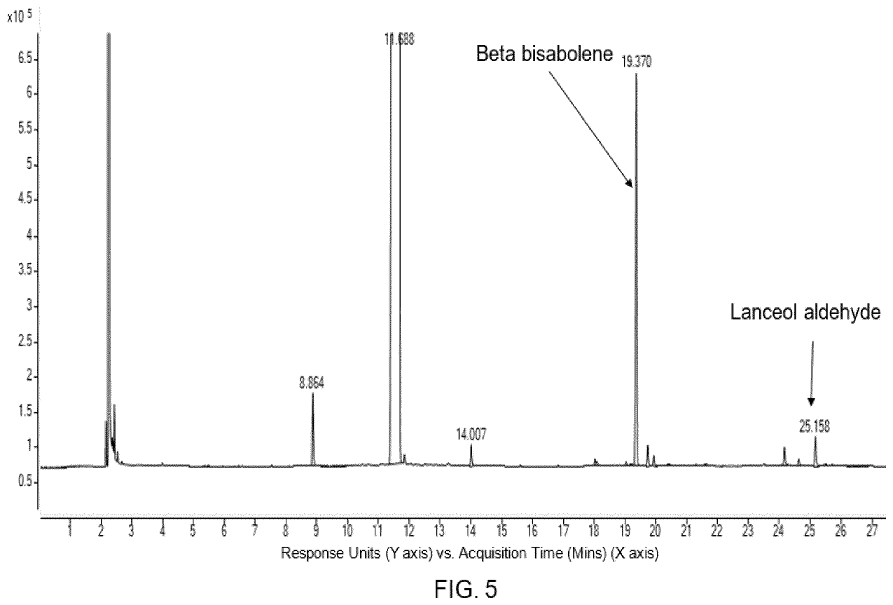

FIG. 5. illustrates Mass spectra demonstrating Bisabolene and lanceol aldehyde production using an alkane oxidation system. See example section for details.

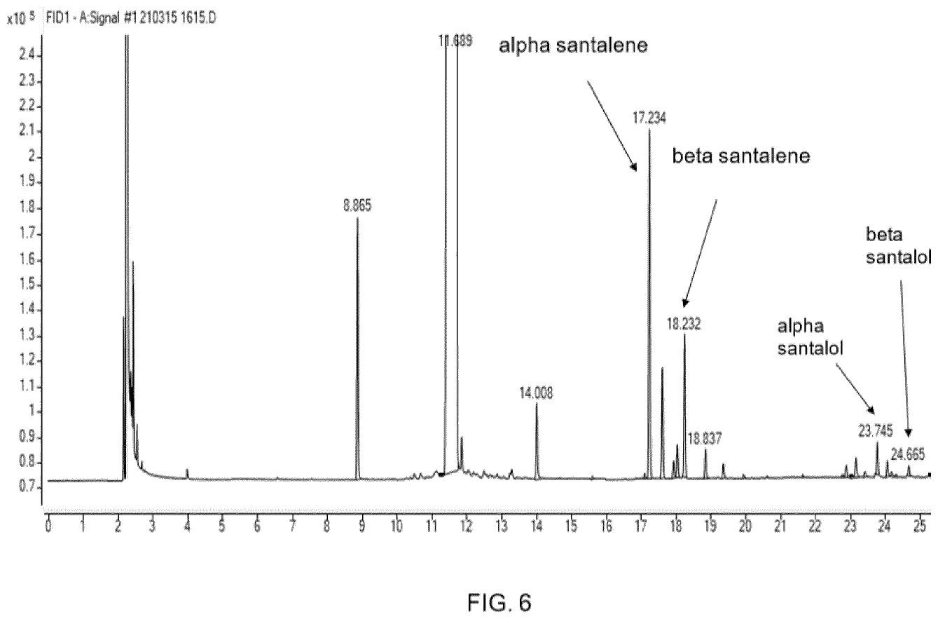

FIG. 6. Illustrates Mass spectra demonstrating Santalene and Santalol were found when an alkane oxidation system was used. See example section for details.

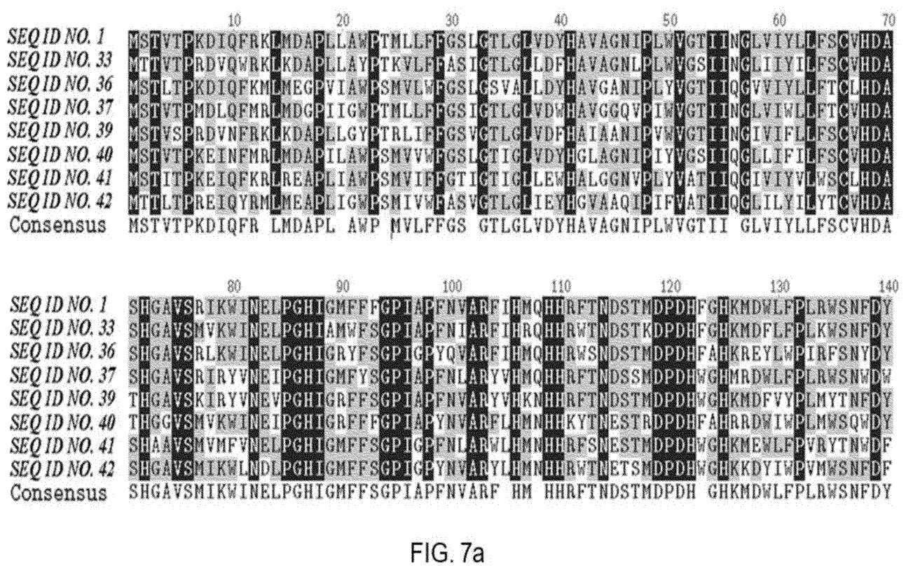

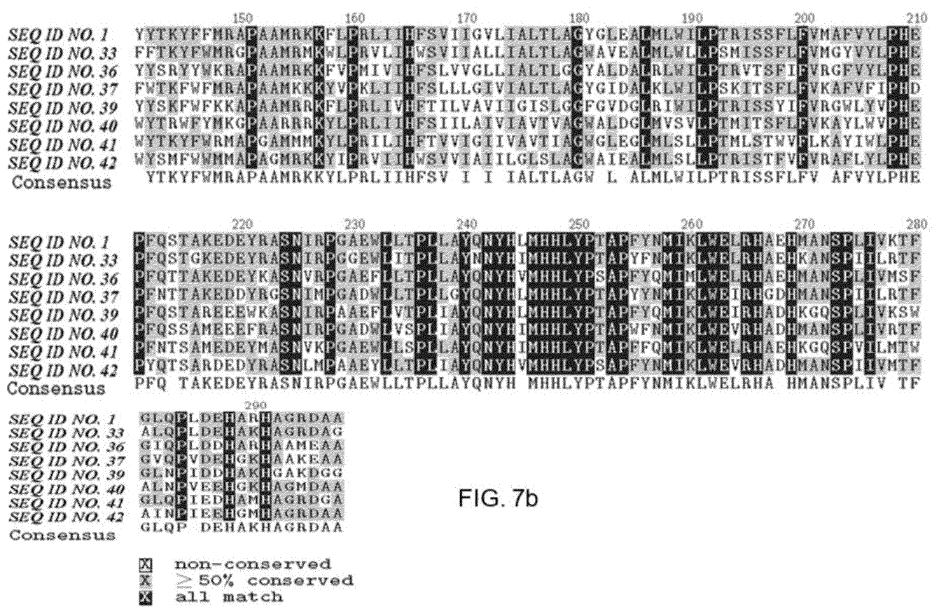

FIGS. 7a & 7b Alignment of the polypeptides of the invention with conserved amino acids at the positions indicated. Conserved amino acid positions are indicated in figures by letters in white font on black background.

### DETAILED DESCRIPTION OF THE INVENTION

Before the present compositions and formulations of the invention are described, it is to be understood that this invention is not limited to particular compositions and formulations described, since such compositions and formulation may, of course, vary. It is also to be understood that the terminology used herein is not intended to be limiting, since the scope of the present invention will be limited only by the appended claims.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”. More specifically, the term “comprise” as used herein means that the claim encompasses all the listed elements or method steps, but may also include additional, unnamed elements or method steps. For example, a method comprising steps a), b) and c) encompasses, in its narrowest sense, a method which consists of steps a), b) and c). The phrase “consisting of” means that the composition (or device, or method) has the recited elements (or steps) and no more. In contrast, the term “comprises” can encompass also a method including further steps, e.g., steps d) and e), in addition to steps a), b) and c).

Furthermore, the terms “first”, “second”, “third” or “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms “first”, “second”, “third” or “(A)”, “(B)” and “(C)” or “(a)”, “(b)”, “(c)”, “(d)”, “i”, “ii” etc. relate to steps of a method or use or assay there is no time or time interval coherence between the steps, that is, the steps may be carried out simultaneously or there may be time intervals of seconds, minutes, hours, days, weeks, months or even years between such steps, unless otherwise indicated in the application as set forth herein above or below.

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “an embodiment” means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment, but may do. Furthermore, the particular features, structures or characteristics may be combined in any suitable manner, as would be apparent to a person skilled in the art from this disclosure, in one or more embodiments. Furthermore, while some embodiments described herein include some, but not other features included in other embodiments, combinations of features of different embodiments are meant to be within the scope of the invention, and form different embodiments, as would be understood by those in the art. For example, in the appended claims, any of the claimed embodiments can be used in any combination.

Furthermore, the ranges defined throughout the specification include the end values as well, i.e. a range of 1 to 10, between 1 to 10 imply that both 1 and 10 are included in the range. For the avoidance of doubt, the applicant shall be entitled to any equivalents according to applicable law.

As used herein, the term “about” when qualifying a value of a stated item, number, percentage, or term refers to a range of plus or minus 10 percent, 9 percent, 8 percent, 7 percent, 6 percent, 5 percent, 4 percent, 3 percent, 2 percent or 1 percent of the value of the stated item, number, percentage, or term. Preferred is a range of plus or minus 10 percent.

In case numerical ranges are used herein such as “in a concentration between 1 and 5 micromolar”, the range includes not only 1 and 5 micromolar, but also any numerical value in between 1 and 5 micromolar, for example, 2, 3 and 4 micromolar.

The term “in vitro” as used herein denotes outside, or external to, the animal or human body. The term “in vitro” as used herein should be understood to include “ex vivo”. The term “ex vivo” typically refers to tissues or cells removed from an animal or human body and maintained or propagated outside the body, e.g., in a culture vessel. The term “in vivo” as used herein denotes inside, or internal to, the animal or human body.

The term “protein” or “polypeptide” or “(poly)peptide” or “peptide” (all terms are used interchangeably, if not indicated otherwise) as used herein encompasses isolated and/or purified and/or recombinant (poly)peptides being essentially free of other host cell polypeptides. The term “peptide” as referred to herein comprises at least two, three, four, five, six, seven, eight, nine, ten, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, 100, 150, 200, 250, 300 or even more amino acid residues where the alpha carboxyl group of one is bound to the alpha amino group of another. A post-translational modification of the protein or peptide as used and envisaged herein is the modification of a newly formed protein or peptide and may involve deletion, substitution or addition of amino acids, chemical modification of certain amino acids, for example, amidation, acetylation, phosphorylation, glycosylation, formation of pyroglutamate, oxidation/reduction of sulfa group on a methionine, or addition of similar small molecules, to certain amino acids.

“Homologues” means bacterial, fungal, plant or animal homologues of the oxidase enzyme or rubredoxin or rubredoxin reductase useful in the invention, preferably plant homologues, but also includes truncated sequences, single-stranded DNA or RNA of the coding and non-coding DNA sequence.

Sequence identity, homology or similarity is defined herein as a relationship between two or more amino acid sequences or two or more nucleic acid sequences, as determined by comparing those sequences. Usually, sequence identities or similarities are compared over the whole length of the sequences, but may also be compared only for a part of the sequences aligning with each other. Preferably, the sequence identities or similarities are compared over the whole length of the sequences, herein. In the art, “identity” or “similarity” also means the degree of sequence relatedness between polypeptide sequences or nucleic acid sequences, as the case may be, as determined by the match between such sequences.

Sequence alignments can be generated with a number of software tools, such as:
Needleman and Wunsch algorithm—Needleman, Saul B. & Wunsch, Christian D. (1970). “A general method applicable to the search for similarities in the amino acid sequence of two proteins”. Journal of Molecular Biology 48 (3): 443-453.

This algorithm is, for example, implemented into the “NEEDLE” program, which performs a global alignment of two sequences. The NEEDLE program, is contained within, for example, the European Molecular Biology Open Software Suite (EMBOSS).
EMBOSS—a collection of various programs: The European Molecular Biology Open Software Suite (EMBOSS), Trends in Genetics 16 (6), 276 (2000).
BLOSUM (BLOcks Substitution Matrix)—typically generated on the basis of alignments of conserved regions, e.g., of protein domains (Henikoff S, Henikoff J G: Amino acid substitution matrices from protein blocks. Proceedings of the National Academy of Sciences of the USA. 1992 Nov. 15; 89(22): 10915-9). One out of the many BLOSUMs is “BLOSUM62”, which is often the “default” setting for many programs, when aligning protein sequences.
BLAST (Basic Local Alignment Search Tool)—consists of several individual programs (BlastP, BlastN) which are mainly used to search for similar sequence in large sequence databases. BLAST programs also create local alignments. Typically used is the “BLAST” interface provided by NCBI(National Centre for Biotechnology Information), which is the improved version (“BLAST2”). The “original” BLAST: Altschul, S. F., Gish, W., Miller, W., Myers, E. W. & Lipman, D. J. (1990) “Basic local alignment search tool.” J. Mol. Biol. 215:403-410; BLAST2: Altschul, Stephen F., Thomas L. Madden, Alejandro A. Schaffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402.

Sequence identity as used herein is preferably the value as determined by the EMBOSS Pairwise Alignment Algorithm “Needle”. In particular, the NEEDLE program from the EMBOSS package can be used (version 2.8.0 or higher, EMBOSS: The European Molecular Biology Open Software Suite—Rice, P., et al. Trends in Genetics (2000) 16: 276-277; http://emboss.bioinformatics.nl) using the NOBRIEF option (‘Brief identity and similarity’ to NO) which calculates the “longest-identity”. The identity, homology or similarity between the two aligned sequences is calculated as follows: Number of corresponding positions in the alignment showing an identical amino acid in both sequences divided by the total length of the alignment after subtraction of the total number of gaps in the alignment. For alignment of amino acid sequences, the default parameters are: Matrix=Blosum62; Open Gap Penalty=10.0; Gap Extension Penalty=0.5. For alignment of nucleic acid sequences, the default parameters are: Matrix=DNAfull; Open Gap Penalty=10.0; Gap Extension Penalty=0.5.

Sequence identity usually is provided as “% sequence identity” or “% identity”. To determine the percent-identity between two amino acid sequences in a first step a pairwise sequence alignment is generated between those two sequences, wherein the two sequences are aligned over their complete, entire or full length (i.e., a pairwise global alignment). The alignment is generated with a program or software described herein. The preferred alignment for the purpose of this invention is that alignment, from which the highest sequence identity can be determined.

An aspect, the present invention is directed to an artificial alkane oxidation system comprising at least one alkane, for example but not limited to an alkene, for example but not limited to a terpene, and as further components:
a. an oxidase enzyme with an amino acid sequence with a sequence identity of at least 62%, 66%, 69%, 70%, 75%, 80%, 85%, 90%, 94%, 95%, 98% or 99% with % with Any one of SEQ ID NO: 1, 10, 11, 23 to 43 or a fragment thereof or a variant thereof, and
b. one or more enzymes providing at least one alkane, preferably a terpene synthase protein,
   optionally with
c. an electron transfer compound suitable to transfer at least one electron to the oxidase enzyme and further optionally
d. an electron transfer compound regeneration enzyme suitable to reduce the electron transfer compound of c. when it is in its oxidized state.

It is possible to have as component a. more than one type of oxidase enzyme.

The artificial alkane oxidation system is kept under conditions suitable for the production of at least one alkane and/or is brought into contact with at least one alkane under conditions suitable to oxidise the said at least one alkane.

In the later case, in one embodiment, the alkane oxidation system can be component a. plus c. and optionally d. but lack b.

An electron transfer compound suitable to transfer at least one electron to the oxidase enzyme (see c. above) may be present in the vicinity of the alkane oxidation system in such a manner that it can functionally interact with the alkane oxidation system although it is not purposely included in the artificial alkane oxidation system. For example, if the alkane oxidation system is included in a host cell, the host cell may provide a suitable electron transfer compound without the need to have one comprised in addition in the alkane oxidation system. Another example could be that cells with a suitable electron transfer compound are disrupted and portions of the cell membrane comprising a suitable electron transfer compound are then combined with an inventive alkane oxidation system that does not comprise itself a suitable electron transfer compound. Typically, it is advantageous to include a suitable electron transfer compound as part of the alkane oxidation system as was also demonstrated.

### Artificial Alkane Oxidation System:

Alkane is to be understood to include alkene. The alkane oxidation system in one embodiment is referred interchangeably to alkene oxidation systems as well.

An “alkene” is to be understood as a hydrocarbon containing a carbon-carbon double bond, preferably a terpene compound. In one aspect of the invention, the alkene is a monoterpene, sesquiterpene or diterpene with one or more allylic groups. In a further aspect of the invention the alkene is a diallylic alkene, for example a diallylic sesquiterpene.

One aspect of the invention hence refers to the artificial alkane oxidation system, the methods of the inventions and the expression cassettes and host cells useful in the methods of the invention, wherein the at least one alkane is made of at least five carbon atoms or has at least one C5, five carbon building block that are present in an integer of 1 to 6, i.e. a carbon atom count of 5, 10, 15, 20, 25 or 30 per alkane molecule, respectively. The at least one alkane includes linear alkenes and non-linear alkenes. In an embodiment, the at least one alkane is a non-linear alkene. In a preferred embodiment the non linear alkene is a terpene. A reference to alkanes preferably is to be understood to refer to alkenes and more preferably to terpenes; accordingly any reference to alkane substrate is preferably a reference to alkene substrate, more preferably to terpene substrate and any reference to an alkane product is preferably a reference to an alkene product, more preferably to a terpene product.

In an embodiment the alkane oxidation system of the invention hence is to be understood to be a terpene oxidation system.

### Component a: Oxidase Enzyme

The disclosed alkane oxidation system includes one or more oxidase enzyme suitable to oxidise at least one alkane, at least one heterologous sesquiterpene synthase protein, optionally rubredoxin peptide, or/and the rubredoxin reductase peptide.

In another aspect of the invention an oxidase enzyme is an enzyme suitable to oxidise at least one alkane and carrying a PFAM domain A_desaturase (PF00487) preferably in its central part (analysed using version 34.0 of PFAM, for PFAM details see “Pfam: The protein families database in 2021: J. Mistry, S. Chuguransky, L. Williams, M. Qureshi, G. A. Salazar, E. L. L. Sonnhammer, S. C. E. Tosatto, L. Paladin, S. Raj, L. J. Richardson, R. D. Finn, A. Bateman Nucleic Acids Research (2020) doi: 10.1093/nar/gkaa913 and http://pfam.xfam.org/).

In one aspect of the invention, an oxidase enzyme is suitable to oxidise at least one alkane and an oxidoreductase of the Enzyme class 1.14.19.x. In a further aspect of the invention the oxidase enzyme is an enzyme of the enzyme class 1.14.19.1.

In one embodiment the oxidase enzyme or the mono-oxygenase enzyme or the alkane oxidising enzyme is defined as an integral membrane di-iron protein. In a further embodiment oxidase enzymes are based on enzymes from bacteria for example but not limited to those that are described further in Smits 2002, J Bacteriol 184, 1733-1742. Alkane oxidizing alkB enzymes have been described from a large range of bacteria (Smits 2002, J Bacteriol 184, 1733-1742). However, no activity of alkB enzymes was demonstrated on farnesene, or any other sesquiterpenes.

In one embodiment, the oxidase enzyme is an alkene monooxygenase from the subclass that is not fused to a rubredoxin naturally. Non-limiting examples are those provided as SEQ ID NO: 1 and 43. The person skilled in the art can determine easily by for example the length of the protein that if it is an alkene monooxygenase of the class fused to rubredoxin by nature, or the alkene monooxygenase class that is not.

Preferably, an oxidase enzyme is an alkane oxidising enzyme with at least 50% identity to the known Pseudomonas M1 alkB hydroxylase (Soares-Castro 2017 Appl Environ Microbiol 83:e03112-16.; SEQ ID NO: 1) for example any one of SEQ ID NO: 23 to 43 or any of the artificial proteins shown in SEQ ID NO: 10, 11 or a fragment or variant thereof. In one embodiment an oxidase enzyme is sourced from Pseudomonas species for example but not limited the Pseudomonas M1 bacterium or based on those enzymes form these organisms. This M1 is able to grow on myrcene, a monoterpene that has structural resemblance to farnesene but lacking one of the isoprene units.

More preferably, an oxidase enzyme is the alkane oxidising alk B enzyme with amino acid sequence having the sequence identity of at least 62%, 66%, 69%, 70%, 75%, 80%, 85%, 90%, 94%, 95%, 98% or 99% with any one of SEQ ID NO: 1, 10, 11 or 23 to 42, (PM1_0216370) for example one from the genus Acinetobacter, as exemplified by the Acinetobacter guillouiae protein disclosed under the Uniprot entry A0A077KZY1 or the Acinetobacter sp. Enzyme disclosed as A0A2S9EQI7 (SEQ ID NO: 23 and 24, respectively). In another more preferred embodiment, the oxidase enzyme of the alkane oxidation system is the alkane oxidising alk B enzyme with amino acid sequence having the sequence identity of at least 62%, 66%, 69%, 70%, 75%, 80%, 85%, 90%, 94%, 95%, 98% or 99% with SEQ ID NO. 43 encoding CMR5c oxidase protein/AlkB oxidase enzyme (Pseudomonas sp. CMR5c). SEQ ID NO: 44 encodes for SEQ ID NO: 43 as well as the rubredoxin of SEQ ID NO:45.

More preferably, an oxidase enzyme is a protein having the amino acid sequence at least 75%, at least 80%, at least 85%, at least 90%, at least 95%, at least 96%, at least 97%, at least 98%, at least 99%, or even 100% sequence identity with any one of SEQ ID NO: 1, 10, 11, 23 to 43 at the amino acid level or a fragment thereof.

In one embodiment, a variant of an oxidase enzyme is a polypeptide of any of SEQ ID NO: 10, 11, 23 to 43 or an amino acid sequence at least 70%, 75%, at least 80%, at least 85%, at least 90%, at least 95%, at least 96%, at least 97%, at least 98% or at least 99% sequence identity with any one of SEQ ID NO: 1, 10, 11, 23 to 43 and having oxidase activity. Preferably a variant is a conservatively modified variant. More preferably, the variant has the conserved amino acids as in FIG. 7a 7b for SEQ ID NO: 1, 33, 36, 37, 39, 40, 41 and 42.

A fragment of an oxidase enzyme as referred to herein may be a polypeptide consisting of any amino acid sequence of the above-mentioned sequences and sequence variants that is of sufficient length of exhibiting an alkane oxidase enzyme activity specified herein.

Typically, a fragment consists of at least 20, at least 30, at least 40, at least 50, at least 100, at least 150 or at least 200 contiguous amino acids in length from the—sequences mentioned herein or sequence variants. In a preferred embodiment a fragment has at least 20%, or at least 30%, or at least 40% or at least 50% or at least 60% or at least 70% or at least 80% or at least 90% or 100% of the activity of the full-length sequence.

On one embodiment of the invention a fragment of an oxidase enzyme is a polypeptide exhibiting an alkane oxidase enzymes activity capable of converting at least one terpene substrate into an oxidised terpene product as defined herein. It is, thus, preferably envisaged that a fragment having the aforementioned biological activity of the polypeptide comprises a PFAM domain A_desaturase (PF00487), preferably in its central part.

### Terpenes:

Per definition, the term “terpenes” comprises the hydrocarbons only, being composed of carbon and hydrogen. The term “terpenoids” refers to terpenes containing additional functional groups, resulting in derivatives such as alcohols, aldehydes, ketones, and acids; see, e.g., Flavours and Fragrances: Chemistry, Bioprocessing and Sustainability RG Berger; Black et al., Terpenoids and their role in wine flavour: recent advances. Australian Journal of Grape and Wine Research 21, 582-600, 2015; Zhou & Pichersky, More is better: the diversity of terpene metabolism in plants. Current Opinion in Plant Biology 2020, 55:1-10; Degenhardt J, Köllner TG, Gershenzon J (2009) Monoterpene and sesquiterpene synthases and the origin of terpene skeletal diversity in plants. Phytochemistry 70(15): 1621-1637). According to the number of isoprene units in their structure which are connected through head-to-tail addition, terpenes are classified according to their number of carbon atoms or sesquiterpenoid moieties, respectively: monoterpenes (C10), sesquiterpenes (C15), diterpenes (C20), triterpenes (C30), or polyterpenes having up to 30,000 connected isoprene units. Just like terpenes, terpenoids are likewise classified according to the number of isoprene units they are constituted of and are further named with the suffix “-oids”, as in monoterpenoids (C10), or in sesquiterpenoids (C15). In the scientific literature, the term terpene is frequently used interchangeably with the term terpenoids, although they have different meanings. As used herein, the term “terpenes”, comprises both hydrocarbons and their functionalized derivatives, preferably hydrocarbons. Typically, the terpene when used as substrate for the inventive alkane oxidation system are not oxygenated forms and hence may not include the oxidised terpene products defined herein elsewhere in the description, although some exceptions like DETAs as defined herein are possible.

“Monoterpenes” as used herein are C10 terpenes. Monoterpenes are typically made from the C10 geranyl diphosphate (GPP) as intermediate and can be cyclic or linear. “Sesquiterpenes” are C15-terpenes built from three isoprene units. Like monoterpenes, sesquiterpenes may be acyclic or contain rings, including many unique combinations. They are found particularly in higher plants and in many other living systems such as marine organisms and fungi. Naturally, they occur as hydrocarbons or in oxygenated forms including lactones, alcohols, acids, aldehydes, and ketones. Sesquiterpenes also include essential oils and aromatic constituents with several pharmacological activities. “Diterpenes” are C-20 terpenes and occur naturally in plants and microbes. Diterpene molecules have commercial value since they can be converted into amber notes, which are applied in the fragrance industries. Typically, sesquiterpenes when used as substrate for the inventive alkane oxidation system are not oxygenated forms and hence may not include the oxidised terpene products defined herein elsewhere in the description, although some exceptions like DETAs as defined herein are possible.

### Component b: Terpene Synthase Protein:

The terpene synthase protein includes terpene synthase proteins have the ability to form one or several terpenes from a single substrate or a number of substrates.

The terpene synthase protein referred hereinbelow include monoterpene, diterpene synthase and/or sesquiterpene synthases. Monoterpene synthase is associated with a common carbocationic reaction mechanism initiated by the divalent metal ion-dependent ionization of a substrate. The resulting cationic intermediate undergoes a series of cyclizations, hydride shifts or other rearrangements until the reaction is terminated by proton loss or the addition of a nucleophile. The monoterpene synthases are described further in the Jorg Degenhardt (Phytochemistry 70 (2009) 1621-1637). Generally, the monoterpene synthase proteins facilitate formation of monoterpenes such as Myrcene, Pinene, Camphene, Phellandrene, Terpinolene, Limonene, Ocimene, Linalool, Cineole, Geraniol, Terpinene, Terpineol, Fenchol, Carene, Sabinene, Bornyl diphosphate, etc and the structural variants, isomers, derivatives, etc. thereof.

Genes encoding diterpene synthases have been extensively described (Zerbe, Trends Biotechnol 2015 July; 33(7):419-28.), and microbial production of these compounds has been demonstrated (e.g. Schalk J. Am. Chem. Soc. 2012, 134, 18900-18903).

Sesquiterpene synthase protein is referred to a protein that facilitate conversion of the acyclic prenyl diphosphates and squalene into a multitude of cyclic and acyclic forms.

Sesquiterpene synthase protein catalyse formation of sesquiterpenes from farnesyl diphosphate employing carbocationic based reaction mechanisms similar as those of monoterpene synthases. Generally, the sesquiterpene synthase proteins facilitate formation of Sesquiterpenes (hereinafter interchangeably referred to as sesquiterpene substrates); these are terpenes with 15 carbon atoms (three isoprene units) such as Longifolene, Farnesene, Bisabolene, Curcumene, Germacrene A/D/D-4-ol, Patchoulol, Valencene, Sesquithujene, Macrocarpene, Caryophyllene, Humulene, Eudesmol, Nerolidol, Barbatene, Amorpha-4,11-diene,8-epi-Cedrol, 5-epi-Aristolochene, Cadinene, Cadinene Vetispiradiene, Bergamotene, Elemene, Cubebene, Cubebol, Muurola-3,5-diene, Selinene, Zingiberene, Farnesol, Sinensal, Santalene, Valencene, guaiene, diterpenes, etc, and the structural variants, isomers, derivatives, etc. thereof.

### Terpene Substrate:

Terpene substrates are to be understood to encompass monoterpene, sesquiterpenes and/diterpenes for example but not limited to terpene hydrocarbon molecules as substrate for the oxidation reaction. In one aspect to the invention the one or more terpene substrate is one or more sesquiterpene substrates. For example the one or more sesquiterpene substrates include includes alpha farnesene, beta farnesene, sinensal, alpha bisabolene, beta bisabolene, alpha bergamotene, beta bergamotene, alpha santalene, beta santalene, valencene, alpha guaiene, diterpenes, monoterpenes, geraniol, nerol monoterpenes, linalyl acetate, limonene, beta-pinene, or geranylactone, or mixtures thereof. Typically, the terpene substrate(s) are not the oxidised terpene products defined herein elsewhere in the description, although some exceptions like DETAs as defined herein are possible.

### Component c: Electron Transfer Compound:

The electron transfer compound transfers at least one electron to the oxidase enzyme.

Preferably, the electron transfer compound includes a) proteins that have soluble electron transfer agents, b) membrane bound components of electron transfer chains, c) mettaloenzymes, etc.

In one embodiment the electron transfer compound is an electron transfer protein, preferably of the F1-S0 type proteins.

More preferably, the electron transfer compound is a rubredoxin peptide with an amino acid sequence with a sequence identity of at least 62%, 66%, 69%, 70%, 75%, 80%, 85%, 90%, 94%, 95%, 98% or 99% with SEQ ID NO: 2 or the one known from UniProt database entry A0A3G7A099 (SEQ ID NO: 45) or a fragment thereof.

The rubredoxin peptide is a soluble low-molecular weight iron-containing peptide needed for electron transfer. Rubredoxin as other non-heme iron proteins like ferredoxin, hemerythrin, aconitase, etc. contain strongly bound functional iron atoms attached to sulfur, but they do not contain porphyrins. The single iron atom is bonded through four tetrahedrally arranged sulfur atoms to the rest of the protein. Electron transfer is handled by a single Fe redox center coordinated to four cysteinyl thiolates. The two redox states are formally Fe(II) and Fe(III).

Preferably, the rubredoxin peptide is an amino acid sequence having the sequence identity of at least 62%, 66%, 69%, 70%, 75%, 80%, 85%, 90%, 94%, 95%, 98% or 99% with SEQ ID NO: 2, or the polypeptide known as A0A3G7A099 from the UniProt database or a fragment thereof.

More preferably, the rubredoxin peptide is the amino acid sequence having at least 75%, at least 80%, at least 85%, at least 90%, at least 95%, at least 96%, at least 97%, at least 98%, at least 99%, or even 100% sequence identity with SEQ ID NO: 2 or 45 at the amino acid level, or a fragment thereof.

A fragment of a rubredoxin peptide as referred to herein may be a polypeptide consisting of any amino acid sequence of the above-mentioned sequences and sequence variants that is of sufficient length of exhibiting an electron transfer activity capable of reducing an oxidised alkane oxidase enzyme to regenerate it.

Typically, a fragment of a rubredoxin peptide consists of at least 20, at least 25, at least 30, at least 40, at least 45 or at least 50 contiguous amino acids in length from the above-mentioned sequences or sequence variants.

### Component d: Electron Transfer Compound Regeneration Enzyme:

Electron transfer compound regeneration enzyme is suitable to reduce the electron transfer compound described herein when it is in its oxidised state and typically to regenerate it.

The electron transfer compound regeneration enzyme is an electron transfer protein reductase, preferably a rubredoxin reductase protein.

The rubredoxin reductase is typically NADH dependent, and needed to recycle the rubredoxin, after electron transfer. In a reaction catalysed by rubredoxin reductase, rubredoxin is reduced by NADH to the ferrous state and reoxidized by the ω-hydroxylase to the ferric form during the catalytic cycle.

Preferably, the rubredoxin reductase peptide is an amino acid sequence having the sequence identity of at least 62%, 66%, 69%, 70%, 75%, 80%, 85%, 90%, 94%, 95%, 98% or 99% with SEQ ID NO: 3, or a fragment thereof.

More preferably, the rubredoxin peptide is the amino acid sequence having at least 75%, at least 80%, at least 85%, at least 90%, at least 95%, at least 96%, at least 97%, at least 98%, at least 99%, or even 100% sequence identity with SEQ ID NO: 3 at the amino acid level, or fragment thereof.

A fragment of a rubredoxin reductase peptide as referred to herein may be a polypeptide consisting of any amino acid sequence of the above-mentioned sequences and sequence variants that is of sufficient length of exhibiting an electron transfer activity capable of reducing an oxidised rubredoxin to regenerate it.

Typically, a fragment of a rubredoxin reductase peptide consists of at least 20, at least 30, at least 40, at least 50, at least 100, at least 150 or at least 200 contiguous amino acids in length from the above-mentioned sequences or sequence variants.

In a preferred embodiment, the artificial alkane oxidation system includes one or more electron transfer proteins as an electron transfer compound, preferably of the F1-S0 type, more preferable one or more rubredoxin peptides with an amino acid sequence with a sequence identity of at least 62%, 66%, 69%, 70%, 75%, 80%, 85%, 90%, 94%, 95%, 98% or 99% with SEQ ID NO: 2 or SEQ ID NO: 45 or a fragment thereof or a variant thereof, or/and at least one electron transfer compound regeneration enzyme is an electron transfer protein reductase, preferably rubredoxin reductase, more preferably a rubredoxin reductase peptide with a sequence identity of at least 62%, 66%, 69%, 70%, 75%, 80%, 85%, 90%, 94%, 95%, 98% or 99% with SEQ ID NO: 3 or a fragment thereof.

In a more preferred embodiment, the alkane oxidation system includes the oxidase enzyme encoded by nucleic acid sequence of sequence identity of at least 70% with SEQ ID NO:20, a rubredoxin peptide encoded by nucleic acid sequence of sequence identity of at least 70% with SEQ ID NO:21, and/or the rubredoxin reductase peptide encoded by nucleic acid sequence of sequence identity of at least 70% with SEQ ID NO:22.

Preferably, the oxidase enzyme is encoded by the nucleic acid sequence with sequence identity of at least 75%, at least 80%, at least 85%, at least 90%, at least 95%, at least 96%, at least 97%, at least 98%, at least 99%, or even 100% sequence identity with SEQ ID NO: 20.

Preferably, the rubredoxin peptide is encoded by nucleic acid sequence with sequence identity of at least 75%, at least 80%, at least 85%, at least 90%, at least 95%, at least 96%, at least 97%, at least 98%, at least 99%, or even 100% sequence identity with SEQ ID NO: 21.

Preferably, the rubredoxin reductase peptide is encoded by nucleic acid sequence with sequence identity of at least 75%, at least 80%, at least 85%, at least 90%, at least 95%, at least 96%, at least 97%, at least 98%, at least 99%, or even 100% sequence identity with SEQ ID NO: 22, or a fragment thereof.

The alkane oxidation system of the invention may be applied in host cells, but also cell free applications are possible. For example, cell membrane portions from disrupted cells, artificial membrane systems, reconstituted cell membranes and the like may be used in combination with the alkane oxidation system of the invention.

Another aspect of the present invention is directed to a synthetic nucleic acid with a sequence identity of at least 62%, 66%, 69%, 70%, 75%, 80%, 85%, 90%, 94%, 95%, 98% or 99% with SEQ ID NO:4, SEQ ID NO:12, SEQ ID NO:17, SEQ ID NO:18, or SEQ ID NO: 19, or SEQ ID NO: 44 wherein the synthetic nucleic acid sequence comprises a nucleic acid sequence encoding the oxidase enzyme, and optionally the rubredoxin peptide and/or the rubredoxin reductase peptide.

Preferably, the synthetic nucleic acid sequence encodes the artificial alkane oxidation system wherein the components listed in brackets are coded for by a nucleic acid sequence of SEQ ID NO: 4 (component a, c and d), SEQ ID NO: 12 (a, c and d), SEQ ID NO: 17 (a, c and d), SEQ ID NO: 18 (a, c and d) or SEQ ID NO: 19 (a and c only).

Preferably, the artificial alkane oxidation system has components a, c and d encoded by the nucleic acid sequence with sequence identity of at least 75%, at least 80%, at least 85%, at least 90%, at least 95%, at least 96%, at least 97%, at least 98%, at least 99%, or even 100% sequence identity with SEQ ID NO: 4, SEQ ID NO: 12, or SEQ ID NO: 17, SEQ ID NO: 18.

Preferably, the artificial alkane oxidation system has components a and c only encoded by the nucleic acid sequence with the nucleic acid sequence has sequence identity of at least 75%, at least 80%, at least 85%, at least 90%, at least 95%, at least 96%, at least 97%, at least 98%, at least 99%, or even 100% sequence identity with SEQ ID NO: 19.

In one embodiment, at least one polypeptide of components of a to d is fused to a tag-peptide. One or more of the polypeptides used in components a to d in one embodiment comprise a first segment comprising a tag-peptide and a second segment comprising the respective polypeptide of the respective component according to the invention. A polypeptide comprising said first and said second segment may herein be referred to as a ‘tagged polypeptide’.

The tag-peptide is preferably selected from the group of nitrogen utilization proteins (NusA), thioredoxins (Trx), maltose-binding proteins (MBP), Glutathione S-transferases (GST), Small Ubiquitin-like Modifier (SUMO) or Calcium-binding proteins (Fh8), and functional homologues thereof. As used herein a functional homologue of a tag peptide is a tag peptide having at least about the same effect on the solubility of the tagged enzyme, compared to the non-tagged enzyme. Typically, the homologue differs in that one or more amino acids have been inserted, substituted, deleted from or extended to the peptide of which it is a homologue. The homologue may in particular comprise one or more substitutions of a hydrophilic amino acid for another hydrophilic amino acid or of a hydrophobic amino acid for another. The homologue may in particular have a sequence identity of at least 40%, more in particular of at least 50%, preferably of at least 55%, more preferably at least 60%, at least 70%, at least 75%, at least 80%, at least 85%, at least 90%, at least 95%, at least 98% or at least 99% with the sequence of a NusA, Trx, MBP, GST, SUMO or Fh8.

In a preferred embodiment, tag-peptide is maltose binding protein from Escherichia coli, or a functional homologue thereof.

The use of a tagged polypeptide according to the invention is in particular advantageous in that it may contribute to an increased production, especially increased cellular production of at least one oxidised terpene product.

For improved solubility of the tagged polypeptide (compared to the polypeptide without the tag), the first segment of the polypeptide is preferably bound at its C-terminus to the N-terminus of the second segment. Alternatively, the first segment of the tagged polypeptide is bound at its N-terminus to the C-terminus of the second segment.

Further, the present disclosure is directed to a nucleic acid comprising a nucleotide sequence encoding a polypeptide, the polypeptide comprising a first segment comprising a tag-peptide, preferably an MBP, a NusA, a Trx, a GST, a SUMO or an Fh8-tag or a functional homologue of any of these, and a second segment comprising a polypeptide of any components a to d. The second segment may for instance comprise an amino acid sequence as shown in SEQ ID NO: 1, 10, 11, or 23 to 43, or a functional analogue thereof.

Further, the present disclosure is directed to a host cell comprising said nucleic acid encoding said tagged polypeptide(s). Specific nucleic acids according to the invention encoding a tagged polypeptide are shown in SEQ ID NO: 4, 12, 17, 18, 19, or 44. The host cell may in particular comprise a gene comprising any of these sequences or a functional analogue thereof.

Further, the present disclosure is directed to a polypeptide, comprising a first segment comprising a tag-peptide and a second segment comprising a polypeptide of any component a to d, the tag-peptide preferably being selected from the group of MBP, NusA, Trx or SET).

The nucleic acids (or polynucleotides) of the invention comprises nucleic acid sequences which encode the alkane oxidation system of the invention. The nucleic acid sequences encoding the alkane oxidation system of the invention are preferably recombinant and/or isolated and/or purified nucleic acid sequences. The nucleic acid sequences which encode the alkane oxidation system of the invention can be produced and isolated using known molecular-biological standard techniques, the sequence information and organisms provided herein. The term “nucleic acid” as used herein, includes reference to a deoxyribonucleotide or ribonucleotide polymer, i.e. a polynucleotide, in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids). A polynucleotide can be full-length or a sub-sequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are “polynucleotides” as the term is used herein. Lt will be appreciated that a great variety of modifications have been made to DNA and RNA that serve many useful purposes known to those of skill in the art. The term “polynucleotide” as it is employed herein embraces such chemically, enzymatically or metabolically modified forms of polynucleotides, as well as the chemical forms of DNA and RNA characteristic of viruses and cells, including among other things, simple and complex cells. Every nucleic acid sequence herein that encodes a polypeptide such as the oxidase enzyme or rubredoxin or rubredoxin reductase also, by reference to the genetic code, describes every possible silent variation of the nucleic acid. The term “conservatively modified variants” applies to both amino acid and nucleic acid sequences. With respect to particular nucleic acid sequences, the term “conservatively modified variants” if used, may refer to those nucleic acids which encode identical or conservatively modified variants of the amino acid sequences due to the degeneracy of the genetic code. The term “degeneracy of the genetic code” refers to the fact that a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. The terms “polypeptide”, “peptide” and “protein” are used interchangeably herein to refer to a polymer of amino acid residues.

The terms “polypeptide”, “peptide” and “protein” apply also to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids. The terms “polypeptide”, “peptide” and “protein” are also inclusive of modifications including, but not limited to, glycosylation, lipid attachment, sulphation, gamma-carboxylation of glutamic acid residues, hydroxylation and ADP-ribosylation. Within the context of the present application, oligomers (such as oligonucleotides, oligopeptides) are considered a species of the group of polymers. Oligomers have a relatively low number of monomeric units, in general 2-100, in particular 6-100, including, e.g., primer sequences, such as used for cloning of the oxidase enzyme or rubredoxin or rubredoxin reductase useful in the invention, in the Examples.

The term “heterologous” when used with respect to a nucleic acid (DNA or RNA) or protein of the invention refers to a nucleic acid or protein that does not occur naturally as part of the organism, cell, genome or DNA or RNA sequence in which it is present, or that is found in a cell or location or locations in the genome or DNA or RNA sequence that differ from that in which it is found in nature. Heterologous nucleic acids or proteins of the invention are not endogenous to the cell into which they are introduced but have been obtained from another cell or synthetically or recombinantly produced. Generally, though not necessarily, such nucleic acids encode proteins that are not normally produced by the cell in which the DNA is expressed. A gene that is endogenous to a particular host cell but has been modified from its natural form, through, for example, the use of DNA shuffling, is also called heterologous. The term “heterologous” also includes non-naturally occurring multiple copies of a naturally occurring DNA sequence. Thus, the term “heterologous” may refer to a DNA segment that is foreign or heterologous to the cell, or homologous to the cell but in a position and/or a number within the host cell nucleic acid in which the segment is not ordinarily found. Exogenous DNA segments are expressed to yield exogenous polypeptides.

A “homologous” DNA sequence of the invention is a DNA sequence that is naturally associated with a host cell into which it is introduced. Any nucleic acid or protein that one of skill in the art would recognize as heterologous or foreign to the cell in which it is expressed is herein encompassed by the term heterologous nucleic acid or protein.

The terms “modified”, “modification”, “mutated”, or “mutation”, as used herein regarding proteins or polypeptides compared to another protein or polypeptide apply mutatis mutandis to nucleotide or nucleic acid sequences. The mentioned terms are used to indicate that the modified nucleotide or nucleic acid sequences encoding the protein or polypeptide has at least one difference in the nucleotide or nucleic acid sequence compared to the nucleotide or nucleic acid sequence of the protein or polypeptide with which it is compared. The terms are used irrespective of whether the modified or mutated protein actually has been obtained by mutagenesis of nucleic acids encoding these amino acids or modification of the polypeptide or protein, or in another manner, e.g. using artificial gene-synthesis methodology. Mutagenesis is a well-known method in the art, and includes, for example, site-directed mutagenesis by means of PCR or via oligonucleotide-mediated mutagenesis, as described in Sambrook, J., and Russell, D. W. Molecular Cloning: A Laboratory Manual. 3d ed., Cold Spring Harbor Laboratory Press, Cold Spring Harbor, NY, (2001). The term “modified”, “modification”, “mutated”, or “mutation” as used herein regarding genes is used to indicate that at least one nucleotide in the nucleotide sequence of that gene or a regulatory sequence thereof, is different from the nucleotide sequence that it is compared. A modification or mutation may in a particular be a replacement of a nucleotide by a different one, a deletion of a nucleotide or an insertion of a nucleotide.

### KIT:

Another aspect of the present invention is directed to a kit for the artificial alkane oxidation system, the kit comprising
a first isolated nucleic acid sequence with a sequence identity of at least 62%, 66%, 69%, 70%, 75%, 80%, 85%, 90%, 94%, 95%, 98% or 99% with SEQ ID NO: 1, 10, 11, 23 to 43 wherein the first nucleic acid sequence comprises a nucleic acid sequence encoding the oxidase enzyme, and
a second isolated nucleic acid sequence encoding an electron transfer protein, preferably not physically connected to the first nucleic acid and
optionally a third nucleic acid sequence encoding an electron transfer protein reductase, wherein the third nucleic acid sequence may be fused to the first and/or second nucleic acid sequence.

In an embodiment, the organism on which the kit is applied includes nucleic acid sequence encoding an alkane synthase, preferably a terpene synthase, more preferably a sesquiterpene synthase as described herein.

In another embodiment, the kit includes a fourth nucleic acid sequence encoding an alkane synthase, preferably a terpene synthase, more preferably a sesquiterpene synthase as described herein. The kit includes the fourth nucleic acid sequence when the organism does not produce the corresponding alkane synthase.

In an embodiment, the kit is used to identify or detect the presence of the artificial alkane oxidation system.

In an alternate embodiment, the kit is used to prepare the artificial alkane oxidation system.

In another aspect, the present invention is directed to an expression cassette comprising:
the synthetic nucleic acid sequence; or
the nucleic acid sequence encoding the alkane oxidation system; or
the nucleic acid sequence(s) of the alkane oxidation system including:
a. a nucleic acid sequence for an oxidase enzyme:
having a sequence identity of at least 70% with SEQ ID NO: 20, or
encoding for an oxidase enzyme with a sequence identity of at least 70% with any one of SEQ ID NO: 1, 10, 11, 23 to 43; and
b. a nucleic acid sequence for a rubredoxin peptide:
having a sequence identity of at least 70% with SEQ ID NO: 21, or
encoding for a rubredoxin of SEQ ID NO: 2 or SEQ ID NO: 45, and optionally
c. a nucleic acid sequence for a rubredoxin reductase peptide:
having a sequence identity of at least 70% with of SEQ ID NO: 22, or
encoding for a rubredoxin reductase peptide of SEQ ID NO: 3.

Another aspect of the present invention is directed to an expression cassette comprising the synthetic nucleic acid sequence of sequence identity of at least 70% with any one of SEQ ID NO: 4, 12, 17, 18 or 19 wherein the synthetic nucleic acid sequence comprises a nucleic acid sequence encoding the oxidase enzyme, a terpene synthase protein if needed, in case the desired terpene synthase function is not available in the intended host cell, and optionally the rubredoxin peptide and/or the rubredoxin reductase peptide.

In another embodiment, the invention is directed to the nucleic acid sequence encoding the alkane oxidation system as disclosed hereinabove.

In another embodiment, the invention is directed to an expression cassette comprising the nucleic acid sequence of the alkane oxidation system, the nucleic acid sequences comprising:
a. a nucleic acid sequence for an oxidase enzyme with a sequence identity of at least 62%, 66%, 69%, 70%, 75%, 80%, 85%, 90%, 94%, 95%, 98% or 99% with any one of SEQ ID NO:20, or encoding for an oxidase enzyme with a sequence identity of at least 62%, 66%, 69%, 70%, 75%, 80%, 85%, 90%, 94%, 95%, 98% or 99% with any one of SEQ ID NO: 1, 10, 11, 23 to 43 and
b. a nucleic acid sequence for a rubredoxin peptide with a sequence identity of at least 62%, 66%, 69%, 70%, 75%, 80%, 85%, 90%, 94%, 95%, 98% or 99% with of any one of SEQ ID NO:21, or
c. a nucleic acid sequence for a rubredoxin reductase peptide with a sequence identity of at least 62%, 66%, 69%, 70%, 75%, 80%, 85%, 90%, 94%, 95%, 98% or 99% with of SEQ ID NO:22 or combination of b and c with a.

Preferably, the nucleic acid sequence for an oxidase enzyme is the nucleic acid sequence having at least 75%, at least 80%, at least 85%, at least 90%, at least 95%, at least 96%, at least 97%, at least 98%, at least 99%, or even 100% sequence identity with SEQ ID NO: 20.

Preferably, the nucleic acid sequence for the rubredoxin peptide is the nucleic acid sequence having at least 75%, at least 80%, at least 85%, at least 90%, at least 95%, at least 96%, at least 97%, at least 98%, at least 99%, or even 100% sequence identity with SEQ ID NO: 21.

Preferably, the nucleic acid sequence for the rubredoxin reductase peptide is the nucleic acid sequence having at least 75%, at least 80%, at least 85%, at least 90%, at least 95%, at least 96%, at least 97%, at least 98%, at least 99%, or even 100% sequence identity with SEQ ID NO: 22.

In one embodiment the artificial alkane oxidation system is a sesquiterpene oxidation system comprising one or more oxidase enzymes with a sequence identity of at least 70, 75, 80, 85, 90, 91, 92, 93, 94, 95, 96, 97, 98 or 99% with any one of SEQ ID NO: 1, 10, 11, 23 to 43, one or more sesquiterpene synthases and optionally a rubredoxin peptide with an amino acid sequence with a sequence identity of at least 70, 75, 80, 85, 90, 91, 92, 93, 94, 95, 96, 97, 98 or 99% with SEQ ID NO: 2, and further optionally a rubredoxin reductase peptide with a sequence identity of at least 70, 75, 80, 85, 90, 91, 92, 93, 94, 95, 96, 97, 98 or 99% with SEQ ID NO: 3.

Another aspect of the present invention is directed to a method of oxidation of one or more alkane substrate, preferable a terpene substrates, more preferably sesquiterpene substrates, the method comprising:
a. providing
   i. the artificial alkane oxidation system components a & c and optionally b and/or d, as disclosed hereinabove, or
   ii. expression of the synthetic nucleic acid sequence of as disclosed hereinabove encoding the oxidase enzyme and optionally the rubredoxin peptide and/or the rubredoxin reductase peptide, or expression cassette thereof as disclosed hereinabove, or expression cassette comprising the artificial oxidation system as disclosed hereinabove,
   iii. expression of a nucleic acid sequence of encoding the oxidase enzyme with an amino acid sequence with sequence identity of at least 70% with any one of SEQ ID NO:1, 10, 11, 23 to 43, or a fragment thereof or a variant thereof, and
   iv. optionally the at least one alkane substrate, preferably, terpene substrate if not produced by the alkane oxidation system—in case a terpene synthase, preferably a sesquiterpene synthase is not present or not functional—a terpene substrate, preferably a sesquiterpene substrate;
   for oxidation of the alkane substrate, preferably, terpene substrate, more preferably sesquiterpene substrate,
b. allylic oxidation of the alkane substrate, preferably, terpene substrate, more preferably sesquiterpene substrate with the alkane oxidation system to produce at least one oxidised alkane product, preferably, terpene product, including one or more aldehyde and/or alcohol products;
c. optionally extracting the at least one oxidised alkane substrate, preferably, terpene product including the one or more aldehyde and/or alcohol products.

In another preferred embodiment of this method of the invention, the at least one oxidised alkane, preferably alkene, more preferably terpene product is prepared in a host cell or a non-human transgenic organism, heterologously expressing the alkane oxidation system components a & c and optionally b (if no endogenous terpene synthase is present) and/or d of the invention.

In another embodiment, the host cell according to the invention can be used industrially in the fermentative production of the aforementioned one or more oxidised terpene products.

For instance, the host cell or non-human transgenic organism of the invention can be used in a fermentative production of the one or more oxidised terpene products.

Preferably, the at least one oxidised terpene product is produced in a fermentative process, i.e. in a method comprising cultivating, e.g., microbial host cells such as Rhodobacter host cells in a culture medium under conditions wherein the alkane oxidation system of the invention is expressed. The actual reaction of allylic oxidation of the one or more terpene substrate(s) with the alkane oxidation system to produce one or more oxidised terpene products takes place typically intracellularly. It should be noted that the term “fermentative” is used herein in a broad sense for processes wherein use is made of a culture of an organism to synthesize a compound from a suitable feedstock (e.g. a carbohydrate, an amino acid source, a fatty acid source). Thus, fermentative processes as meant herein are not limited to anaerobic conditions, and extended to processes under aerobic conditions. Suitable feedstocks are generally known for Rhodobacter host cells. Suitable conditions can be based on known methodology for Rhodobacter host cells, e.g. described in WO 2011/074954 or in WO 2014/014339.

The at least one oxidised terpene product produced by oxidation of the one or more terpene substrates can be isolated or extracted from the host cell or non-human transgenic organism by methods known in the art (for plants, see, e.g., Jiang et al., Curr Protoc Plant Biol. 2016; 1: 345-358. Doi:10.1002/cppb.20024; for Rhodobacter, see, e.g., WO 2014/014339).

In general, the methods include the following steps: 1) breaking the cells to release their chemical constituents including the oxidised terpene products; 2) extracting the sample including the oxidised terpene products using a suitable solvent (or through distillation or the trapping of compounds); 3) separating the desired oxidised terpene product from other undesired contents of the extracts that confound analysis and quantification; and 4) use an appropriate method of analysis (e.g., thin layer chromatography (TLC), gas chromatography (GC), or liquid chromatography (LC) or another method as described herein).

In an embodiment, the oxidised terpene product is produced extracellularly and the extraction proceeds with extraction of the sample using suitable solvent.

### Terpene Substrate:

Terpene substrates are defined hereinabove.

Preferably, the one or more terpene substrates includes alpha farnesene, beta farnesene, alpha bisabolene, beta bisabolene, alpha bergamotene, beta bergamotene, alpha santalene, beta santalene, valencene, alpha guaiene, diterpenes, monoterpenes, geraniol, nerol monoterpenes, or geranylacetone, linalyl acetate, limonene, beta-pinene, or mixtures thereof.

In an embodiment, at least one terpene substrate is a sesquiterpene substrate selected from alpha farnesene, beta farnesene, alpha bisabolene, beta bisabolene, alpha bergamotene, beta bergamotene, alpha santalene, beta santalene, valencene, or alpha guaiene.

In one embodiment, the terpene substrates include one or more distant end terpene alcohol. Distant end terpene alcohol (DETA) carry an alcohol group at the end of the carbon chain that is considered the distant end of the terpene before oxidation to the alcohol. Non-limiting examples for these are for the terpene myrcene the compound of formula I and for the terpene beta-farnesene the compounds shown in formula II and III below:

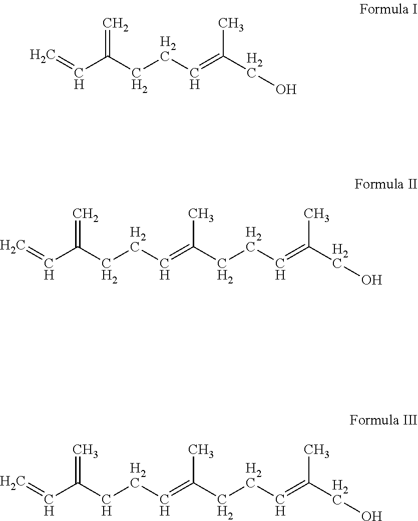 [SMILES: C=CC(=C)CC/C=C(\C)CO ; C=CC(=C)CC/C=C(\C)CC/C=C(\C)CO ; C=CC(=C)CC/C=C(\C)CC/C=C(\C)CO]

### Oxidised Terpene Product:

The at least one oxidised terpene product is produced by oxidation, preferably by allylic oxidation, of the terpene substrate.

Preferably, the at least one oxidised terpene product is terpene based and is produced by the oxidation of the one or more terpene substrates. They may comprise aldehyde groups, alcohol groups and/or allylic alcohol groups introduced through the action of the alkane oxidation system components a and c and optionally d. In one embodiment the oxidised terpene products include at least one of the following: myrcene aldehyde, alpha sinensal, beta sinensal, trans alpha santalol and trans beta santalol, lanceol-aldehyde, nootkatone, vetivone, rotundone, rebaudiosides, 8-hydroxygeraniol, 8-hydroxynerol, 9,10-epoxygeranylacetone. Hexadecenal, farnesol, denderalasin and bicyclo-octanediol, oxidised alpha guaiene, oxidized beta guaiene.

In one embodiment, the oxidised terpene product(s) include distant end terpene alcohol DETA as defined above. DETAs can be intermediates on the oxidation of the corresponding terpenes to oxidised terpene products such as but not limited to myrcene aldehyde, alpha sinensal, beta sinensal, trans alpha santalol and trans beta santalol, lanceol-aldehyde, nootkatone, vetivone, rotundone, rebaudiosides, 8-hydroxygeraniol, 8-hydroxynerol, or 9,10-epoxygeranylacetone. DETAs can itself be the oxidised terpene product when the oxidation is not continued to the oxidised terpene products of a greater oxidation level, e.g. to aldehydes. As described above DETAs may have a dual function, as the alkane oxidation system of the invention can in one embodiment use them as terpen substrates to produce oxidised terpen substrates of a greater oxidation level, or the DETAs can be the product of the alkane oxidation system of the invention.

In an embodiment, the monoterpene substrate myrcene is used to produce myrcene aldehyde.

In another embodiment, the sesquiterpene substrate alpha-farnescene and beta-farnescene is used to produce alpha sinensal and beta sinensal respectively.

In another embodiment, the sesquiterpene substrate santalene is used to produce santalol.

In another embodiment, the sesquiterpene substrate valencene is used to produce nootkatone or vetivone.

In another embodiment, the sesquiterpene substrate alpha guaiene is used to produce rotundone.

In another embodiment, the sesquiterpene substrate diterpene is used to produce rebaudioside.

In another embodiment, the sesquiterpene substrate geraniol or nerol monoterpenes are used to produce 8-hydroxygeraniol or 8-hydroxynerol.

In another embodiment, the geranylacetone is used for production of 9, 10-epoxygeranylacetone.

In a preferred embodiment, the at least one alkane substrate is selected from diterpenes, monoterpenes or sesquiterpenes, including alpha farnesene, beta farnesene, alpha bisabolene, beta bisabolene, alpha bergamotene, beta bergamotene, alpha santalene, beta santalene, valencene, alpha guaiene, monoterpene geraniol, nerol monoterpenes, geranylactone, linalyl acetate, limonene, beta-pinene, or combination thereof.

In another aspect, the present invention is directed to a method of producing at least one oxidised alkane product by oxidation of an alkane substrate.

Another aspect of the invention is directed to non-human host cell. A non-human host cell comprises:

the artificial alkane oxidation system, or
the synthetic nucleic acid, the expression cassettes, or
oxidation of the alkane substrate, or
production of the alkane oxidized product.

The non-human host cell is suitable for oxidation of the terpene substrate as disclosed hereinabove.

The non-human host cell are non-mammalian cells, preferably non-vertebrate cells, more preferably isolated host cells.

Preferably, the transgenic non-human organism of the invention is a bacterium, a yeast, a fungus, a protist, an algae or a cyanobacteria, a non-human animal or a non-human mammalian, or a plant.

The non-host cell includes a bacterial, fungal, etc. host cell.

The invention also relates to a vector or gene construct comprising the nucleic acid of the invention.

The nucleic acid of the invention is operatively linked to expression control sequences allowing expression in prokaryotic or eukaryotic host cells, or isolated fractions thereof, in a vector or gene construct. Thus, in an aspect, the vector is an expression vector. Expression of the nucleic acid of the invention comprises transcription of the polynucleotide into a translatable mRNA. Regulatory elements ensuring expression in prokaryotic or eukaryotic host cells are well known in the art. In an aspect, they comprise regulatory sequences ensuring initiation of transcription and/or poly-A signals ensuring termination of transcription and stabilization of the transcript. Additional regulatory elements may include transcriptional as well as translational enhancers. Possible regulatory elements permitting expression in prokaryotic host cells comprise, e.g., the lac-, trp- or tac-promoter in E. coli, or Rhodobacter promoters (https://doi.org/10.1073/pnas.2010087117), and examples for regulatory elements permitting expression in eukaryotic host cells are the AOX1- or the GAL1-promoter in yeast or the CMV-, SV40-, RSV-promoter (Rous sarcoma virus), CMV-enhancer, SV40-enhancer or a globin intron in mammalian and other animal cells. Plant promoters are described, e.g., in Plant Biotechnology: Principles and Applications, pp 117-172, 2017. Moreover, inducible expression control sequences may be used in an expression vector. Such inducible vectors may comprise tet or lac operator sequences or sequences inducible by heat shock or other environmental factors. Suitable expression control sequences are well known in the art. Beside elements which are responsible for the initiation of transcription such regulatory elements may also comprise transcription termination signals, such as the SV40-poly-A site or the tk-poly-A site, downstream of the polynucleotide. In this context, suitable expression vectors are known in the art, such as Okayama-Berg cDNA expression vector pcDV1 (Pharmacia), pBluescript (Stratagene), pCDM8, pRc/CMV, pcDNA1, pcDNA3 (Invitrogen) or pSPORT1 (Invitrogen). Expression vectors derived from viruses such as retroviruses, vaccinia virus, adeno-associated virus, herpes viruses, or bovine papilloma virus, may be used for delivery of the polynucleotide or vector into a targeted cell population.

Methods which are well known to those skilled in the art can be used to construct vectors or gene constructs comprising the nucleic acid of the invention; see, for example, the techniques described in Sambrook, Molecular Cloning A Laboratory Manual, Cold Spring Harbor Laboratory (2001) N. Y. and Ausubel, Current Protocols in Molecular Biology, Green Publishing Associates and Wiley Interscience, N.Y. (1994).

The term “gene” as used herein is used broadly to refer to any segment of nucleic acid associated with a biological function, such as the nucleic acid of the invention. Thus, genes include coding sequences and/or the regulatory sequences required for their expression. For example, gene refers to a nucleic acid fragment that expresses mRNA or functional RNA, or encodes a specific protein, and which includes regulatory sequences. Genes also include non-expressed DNA segments that, for example, form recognition sequences for other proteins. Genes can be obtained from a variety of sources, including cloning from a source of interest or synthesizing from known or predicted sequence information, and may include sequences designed to have desired parameters.

The term “chimeric gene” as used herein refers to any gene that contains 1) DNA sequences, including regulatory and coding sequences that are not found together in nature, or 2) sequences encoding parts of proteins not naturally adjoined, or 3) parts of promoters that are not naturally adjoined. Accordingly, a chimeric gene may comprise regulatory sequences and coding sequences that are derived from different sources, or comprise regulatory sequences and coding sequences derived from the same source, but arranged in a manner different from that found in nature.

A “gene construct” as used herein can vary in complexity according to the insertion of interest. The construct can be designed to be inserted randomly into the genome of an organism, which is called transgenesis by addition, or can be designed to be inserted into the genome at a specific targeted site, into the correct position of a determined chromosome, which is called transgenesis by homologous recombination. In both cases, the construct must be impeccable, with structures to control gene expression, such as a promoter, a site of transcription initiation, a site of polyadenylation, and a site of transcription termination. That is, the information which is being inserted into the receptor genome has a beginning, middle, and an end, thus avoiding problems of uncontrolled expression in the host cell or organism.

The terms “open reading frame” and “ORF” as used herein refer to the amino acid sequence encoded between translation initiation and termination codons of a coding sequence. The terms “initiation codon” and “termination codon” refer to a unit of three adjacent nucleotides (‘codon’) in a coding sequence that specifies initiation and chain termination, respectively, of protein synthesis (mRNA translation).

“Coding sequence” as used herein refers to a DNA or RNA sequence that codes for a specific amino acid sequence and excludes the non-coding sequences. Lt may constitute an “uninterrupted coding sequence”, i.e. lacking an intron, such as in a cDNA or it may include one or more introns bound by appropriate splice junctions. An “intron” is a sequence of RNA which is contained in the primary transcript but which is removed through cleavage and re-ligation of the RNA within the cell to create the mature mRNA that can be translated into a protein.

“Regulatory sequences” as used herein refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences which may be a combination of synthetic and natural sequences. As is noted above, the term “suitable regulatory sequences” is not limited to promoters. Examples of regulatory sequences include promoters (such as transcriptional promoters, constitutive promoters, inducible promoters), operators, enhancers, mRNA ribosomal binding sites, and appropriate sequences which control transcription and translation initiation and termination. Nucleic acid sequences are “operably linked” when the regulatory sequence functionally relates to the DNA or cDNA sequence of the invention. As used herein, the term “operably linked” or “operatively linked” refers to a juxtaposition wherein the components so described are in a relationship permitting them to function in their intended manner. A control sequence “operably linked” to another control sequence and/or to a coding sequence is ligated in such a way that transcription and/or expression of the coding sequence is achieved under conditions compatible with the control sequence. Generally, operably linked means that the nucleic acid sequences being linked are contiguous and, where necessary to join two protein coding regions, contiguous and in the same reading frame. Each of the regulatory sequences may independently be selected from heterologous and homologous regulatory sequences.

“Promoter” as used herein refers to a nucleotide sequence, usually upstream (5′) to its coding sequence, which controls the expression of said coding sequence by providing the recognition for RNA polymerase and other factors required for proper transcription. “Promoter” includes a minimal promoter that is a short DNA sequence comprised of a TATA box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence which can stimulate promoter activity and may be an innate element of the promoter or a heterologous element inserted to enhance the level or tissue specificity of a promoter. Lt is capable of operating in both orientations (normal or flipped), and is capable of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements derived from different promoters found in nature, or even be comprised of synthetic DNA segments. A promoter may also contain DNA sequences that are involved in the binding of protein factors which control the effectiveness of transcription initiation in response to physiological or developmental conditions.

“Expression cassette” as used herein means a DNA sequence capable of directing expression of a particular nucleotide sequence, for example, a nucleotide sequence encoding the oxidase enzyme and optionally rubredoxin and optionally rubredoxin reductase useful in the invention, in an appropriate host cell as defined herein, comprising a promoter operably linked to the nucleotide sequence of interest which is operably linked to termination signals. Lt also typically comprises sequences required for proper translation of the nucleotide sequence. The coding region usually codes for a protein of interest but may also code for a functional RNA of interest, for example, antisense RNA or a non-translated RNA, in the sense or antisense direction. The expression cassette comprising the nucleotide sequence of interest may be chimeric, meaning that at least one of its components is heterologous with respect to at least one of its other components. The expression cassette may also be one which is naturally occurring but has been obtained in a recombinant form useful for heterologous expression. The expression of the nucleotide sequence in the expression cassette may be under the control of a constitutive promoter or of an inducible promoter which initiates transcription only when the host cell is exposed to some particular external stimulus. In the case of a multicellular organism, the promoter can also be specific to a particular tissue or organ or stage of development, e.g. in plant development.

The term “vector” as used herein refers to a construction comprised of genetic material designed to direct transformation of a targeted cell. A vector contains multiple genetic elements positionally and sequentially oriented, i.e., operatively linked with other necessary elements such that the nucleic acid in a nucleic acid cassette can be transcribed and when necessary, translated in the transformed cells. In particular, the vector may be selected from the group of viral vectors, (bacterio)phages, cosmids or plasmids. The vector may also be a yeast artificial chromosome (YAC), a bacterial artificial chromosome (BAC) or Agrobacterium binary vector. The vector may be in double or single stranded linear or circular form which may or may not be self-transmissible or mobilizable, and which can transform host organisms such as, e.g., Rhodobacter either by integration into the cellular genome or exist extra chromosomally (e.g. autonomous replicating plasmid with an origin of replication). Specifically included are shuttle vectors by which is meant a DNA vehicle capable, naturally or by design, of replication in two different host organisms as defined herein. Preferably, the nucleic acid in the vector is under the control of, and operably linked to, an appropriate promoter or other regulatory elements for transcription in a host cell as specified herein. The vector may be a bi-functional expression vector which functions in multiple hosts. In the case of genomic DNA, this may contain its own promoter or other regulatory elements and in the case of cDNA this may be under the control of an appropriate promoter or other regulatory elements for expression in the host cell. Vectors containing a nucleic acid can be prepared based on methodology known in the art. For instance, use can be made of a cDNA sequence encoding the oxidase enzyme or rubredoxin or rubredoxin reductase useful in the invention operably linked to suitable regulatory elements, such as transcriptional or translational regulatory nucleic acid sequences.

The term “vector” as used herein, includes reference to a vector for standard cloning work (“cloning vector”) as well as to more specialized type of vectors, like an (autosomal) expression vector and a cloning vector used for integration into the chromosome of the host cell (“integration vector”).

“Cloning vectors” typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector.

The term “expression vector” as used herein refers to a DNA molecule, linear or circular, that comprises a segment encoding a polypeptide of interest under the control of (i.e. operably linked to) additional nucleic acid segments that provide for its transcription. Such additional segments may include promoter and terminator sequences, and may optionally include one or more origins of replication, one or more selectable markers, an enhancer, a polyadenylation signal, and the like. Expression vectors are generally derived from plasmid or viral DNA, or may contain elements of both. In particular, an expression vector comprises a nucleotide sequence that comprises in the 5′ to 3′ direction and operably linked: (a) a transcription and translation initiation region that are recognized by the host organism, (b) a coding sequence for a polypeptide of interest, and (c) a transcription and translation termination region that are recognized by the host organism. “Plasmid” refers to autonomously replicating extrachromosomal DNA which is not integrated into a microorganism's genome and is usually circular in nature.

An “integration vector” refers to a DNA molecule, linear or circular, that can be incorporated, e.g., into a microorganism's genome, such as a bacteria's genome, and provides for stable inheritance of a gene encoding a polypeptide of interest, such as the alkane oxidation system, for example oxidase enzyme and terpene synthase if needed, and optionally rubredoxin and optionally rubredoxin reductase useful the invention. The integration vector generally comprises one or more segments comprising a gene sequence encoding a polypeptide of interest under the control of (i.e., operably linked to) additional nucleic acid segments that provide for its transcription.

Such additional segments may include promoter and terminator sequences, and one or more segments that drive the incorporation of the gene of interest into the genome of the target cell, usually by the process of homologous recombination. Typically, the integration vector will be one which can be transferred into the target cell, but which has a replicon which is non-functional in that organism. Integration of the segment comprising the gene of interest may be selected if an appropriate marker is included within that segment. One or more nucleic acid sequences encoding appropriate signal peptides that are not naturally associated with a polypeptide to be expressed in a host cell of the invention can be incorporated into (expression) vectors. For example, a DNA sequence for a signal peptide leader can be fused in-frame to a nucleic acid of the invention so that the oxidase enzyme or rubredoxin or rubredoxin reductase useful in the invention is initially translated as a fusion protein comprising the signal peptide. Depending on the nature of the signal peptide, the expressed polypeptide will be targeted differently. A secretory signal peptide that is functional in the intended host cells, for instance, enhances extracellular secretion of the expressed polypeptide. Other signal peptides direct the expressed polypeptide to certain organelles, like the chloroplasts, mitochondria and peroxisomes. The signal peptide can be cleaved from the polypeptide upon transportation to the intended organelle or from the cell. Lt is possible to provide a fusion of an additional peptide sequence at the amino or carboxyl terminal end of the polypeptide.

In addition, the invention concerns a host cell comprising the vector or gene construct of the invention.

The host cell is transformed with the vector or gene construct of the invention. The skilled artisan is well aware of the genetic elements that must be present on the genetic construct to successfully transform, select and propagate host cells containing the vector or gene construct of the invention. The host cell of the invention is capable of expressing the polypeptide(s) of the alkane oxidation system, included in the vector or gene construct of the invention.

“Transformation” and “transforming”, as used herein, refers to the introduction of a heterologous nucleotide sequence, such as the nucleotide sequence encoding the alkane oxidation system, for example oxidase enzyme and optionally rubredoxin and optionally rubredoxin reductase useful in the invention, into a host cell, irrespective of the method used for the insertion, for example, direct uptake, transduction, conjugation, f-mating or electroporation. The exogenous polynucleotide may be maintained as a non-integrated vector, for example, a plasmid, or alternatively, may be integrated into the host cell genome.

A host cell according to the invention may be produced based on standard genetic and molecular biology techniques that are generally known in the art, e.g., as described in Sambrook, J., and Russell, D. W. “Molecular Cloning: A Laboratory Manual” 3d ed., Cold Spring Harbor Laboratory Press, Cold Spring Harbor, N Y, (2001); and F. M. Ausubel et al, eds., “Current protocols in molecular biology”, John Wiley and Sons, Inc., New York (1987), and later supplements thereto.

The host cell can be any cell selected from a microbial cell, e.g., a bacterial cell, an archaeal cell, a fungal cell, such as a yeast cell, and a protist cell. The host cell can also be an algal cell or a cyanobacterial cell, a non-human animal cell or a mammalian cell, or a plant cell.

Specifically, the host cell can be selected from any one of the following organisms:

### Bacteria:

The bacterial host cell can, for example, be selected from the group consisting of the genera Escherichia, Klebsiella, Helicobacter, Bacillus, Lactobacillus, Streptococcus, Amycolatopsis, Rhodobacter, Pseudomonas, Paracoccus, Pantoea or Lactococcus.

### Gram Positive: Bacillus, Streptomyces:

Useful gram positive bacterial host cells include, but are not limited to, a Bacillus cell, e.g., Bacillus alkalophius, Bacillus amyloliquefaciens, Bacillus brevis, Bacillus circulans, Bacillus clausii, Bacillus coagulans, Bacillus firmus, Bacillus Jautus, Bacillus lentus, Bacillus licheniformis, Bacillus megaterium, Bacillus pumilus, Bacillus stearothermophilus, Bacillus subtilis, and Bacillus thuringiensis. Most preferred, the prokaryote is a Bacillus cell, preferably, a Bacillus cell of Bacillus subtilis, Bacillus pumilus, Bacillus licheniformis, or Bacillus lentus.

Some other preferred bacteria include strains of the order Actinomycetales, preferably, Streptomyces, preferably Streptomyces spheroides (ATTC 23965), Streptomyces thermoviolaceus (IFO 12382), Streptomyces lividans or Streptomyces murinus or Streptoverticillum verticillium ssp. verticillium. Other preferred bacteria include Rhodobacter sphaeroides, Rhodomonas palustri, Streptococcus lactis. Further preferred bacteria include strains belonging to Myxococcus, e.g., M. virescens.

Gram Negative: E. coli, Pseudomonas, Rhodobacter, Paracoccus or Pantoea sp.

Preferred gram negative bacteria are Escherichia coli, Pseudomonas sp., preferably, Pseudomonas purrocinia (ATCC 15958) or Pseudomonas fluorescens (NRRL B-11), Rhodobacter capsulatus or Rhodobacter sphaeroides, Paracoccus carotinifaciens or Paracoccus zeaxanthinifaciens) or Pantoea ananatis.

### Fungi:

Aspergillus, Fusarium, Trichoderma

The host cell may be a fungal cell. “Fungi” as used herein includes the phyla Ascomycota, Basidiomycota, Chytridiomycota, and Zygomycota as well as the Oomycota and Deuteromycotina and all mitosporic fungi. Representative groups of Ascomycota include, e.g., Neurospora, Eupenicillium (=Penicillium), Emericella (=Aspergillus), Eurotium (=Aspergillus), and the true yeasts listed below. Examples of Basidiomycota include mushrooms, rusts, and smuts. Representative groups of Chytridiomycota include, e.g., Allomyces, Blastocladiella, Coelomomyces, and aquatic fungi. Representative groups of Oomycota include, e.g. Saprolegniomycetous aquatic fungi (water molds) such as Achlya. Examples of mitosporic fungi include Aspergillus, Penicillium, Candida, and Alternaria. Representative groups of Zygomycota include, e.g., Rhizopus and Mucor.

Some preferred fungi include strains belonging to the subdivision Deuteromycotina, class Hyphomycetes, e.g., Fusarium, Humicola, Tricoderma, Myrothecium, Verticillum, Arthromyces, Caldariomyces, Ulocladium, Embellisia, Cladosporium or Dreschlera, in particular Fusarium oxysporum (DSM 2672), Humicola insolens, Trichoderma resii, Myrothecium verrucana (IFO 6113), Verticillum alboatrum, Verticillum dahlie, Arthromyces ramosus (FERM P-7754), Caldariomyces fumago, Ulocladium chartarum, Embellisia alli or Dreschlera halodes.

Other preferred fungi include strains belonging to the subdivision Basidiomycotina, class Basidiomycetes, e.g. Coprinus, Phanerochaete, Coriolus or Trametes, in particular Coprinus cinereus f. microsporus (IFO 8371), Coprinus macrorhizus, Phanerochaete chrysosporium (e.g. NA-12) or Trametes (previously called Polyporus), e.g. T. versicolor (e.g. PR4 28-A).

Further preferred fungi include strains belonging to the subdivision Zygomycotina, class Mycoraceae, e.g. Rhizopus or Mucor, in particular Mucor hiemalis.

### Yeast:

Pichia

Saccharomyces

The fungal host cell may be a yeast cell. Yeast as used herein includes ascosporogenous yeast (Endomycetales), basidiosporogenous yeast, and yeast belonging to the Fungi Imperfecti (Blastomycetes). The ascosporogenous yeasts are divided into the families Spermophthoraceae and Saccharomycesaceae. The latter is comprised of four subfamilies, Schizosaccharomycoideae (e.g., genus Schizosaccharomyces), Nadsonioideae, Lipomycoideae, and Saccharomycoideae (e.g. genera Kluyveromyces, Pichia, and Saccharomyces). The basidiosporogenous yeasts include the genera Leucosporidim, Rhodosporidium, Sporidiobolus, Filobasidium, and Filobasidiella. Yeasts belonging to the Fungi Imperfecti are divided into two families, Sporobolomycetaceae (e.g., genera Sporobolomyces and Bullera) and Cryptococcaceae (e.g. genus Candida).

### Eukaryotes:

Eukaryotic host cells further include, without limitation, a non-human animal cell, a non-human mammal cell, an avian cell, reptilian cell, insect cell, or a plant cell.

In a preferred embodiment, the host cell is a host cell selected from:
a) a bacterial cell of the group of Gram negative bacteria, such as Rhodobacter (e.g. Rhodobacter sphaeroides or Rhodobacter capsulatus), Paracoccus (e.g. P. carotinifaciens, P. zeaxanthinifaciens), Escherichia or Pseudomonas;
b) a bacterial cell selected from the group of Gram positive bacteria, such as Bacillus, Corynebacterium, Brevibacterium, Amycolatopis;
c) a fungal cell selected from the group of Aspergillus, Blakeslea, Peniciliium, Phaffia (Xanthophyllomyces), Pichia, Saccharamoyces, Kluyveromyces, Yarrowia, and Hansenula;
d) a transgenic plant cell or a culture comprising transgenic plant cells, wherein the cell is of a transgenic plant selected from Arabidopsis spp., Nicotiana spp, Cichorum intybus, lacuca sativa, Mentha spp, Artemisia annua, tuber forming plants, oil crops, e.g. Brassica spp. Or Brassica napus, flowering plants (angiosperms) which produce fruits, and trees; or e) a transgenic mushroom or culture comprising transgenic mushroom cells, wherein the microorganism is selected from Schizophyllum, Agaricus and Pleurotisi.

More preferred host cells from organisms are host cells from microorganisms belonging to the genus Escherichia, Saccharomyces, Pichia, Rhodobacter, Pseudomonas, Pantoea or Paracoccus, (e.g. Paracoccus carotinifaciens, Paracoccus zeaxanthinifaciens) and even more preferred those of the species E. coli, S. cerevisae, Rhodobacter sphaeroides, Rhodobacter capsulatus, Pantoea ananatis or Amycolatopis sp.

Preferably, the vector or gene construct is suitable for encoding and producing the alkane oxidation system components a & c and optionally b and/or d of the invention, in a microbial cell as defined herein.

In one embodiment the host cell is a Rhodobacter host cell selected from the group of Rhodobacter capsulatus and Rhodobacter sphaeroides.

The transgenic non-human organism of the invention comprises the nucleic acid of the invention, the vector or gene construct of the invention, or the host cell of the invention. In a preferred embodiment, the transgenic non-human organism of the invention is used for preparing one or more oxidised terpene products as defined herein such as—but not limited to—alpha sinensal, beta sinensal, trans alpha santalol and trans beta santalol, lanceol-aldehyde, nootkatone, vetivone, rotundone, rebaudiosides, 8-hydroxygeraniol, 8-hydroxynerol, or 9,10-epoxygeranylacetone, as described in more detail elsewhere in this specification. The one or more oxidised terpene products is prepared by the allylic oxidation of the terpene substrate with the alkane oxidation system of the invention.

Preferably, the transgenic non-human organism of the invention is a bacteria, a yeast, a fungus, a protist, an algae or a cyanobacteria, a non-human animal or a non-human mammalian, or a plant. Specifically, the organisms mentioned in connection with host cells of the invention can also be used for the generation of a transgenic non-human organism of the present invention.

It is preferred that the bacteria is a Gram negative bacteria, preferably Rhodobacter, Escherichia, Pseudomonas Pantoea or Paracoccus.

The term “transgenic” for a transgenic organism or transgenic cell as used herein, refers to an organism or cell (which cell may be an organism per se or a cell of a multi-cellular organism from which it has been isolated) containing a nucleic acid not naturally occurring in that organism or cell and which nucleic acid has been introduced into that organism or cell (i.e., has been introduced in the organism or cell itself or in an ancestor of the organism or an ancestral organism of an organism of which the cell has been isolated) using recombinant DNA techniques known in the art. Or to put it differently: The nucleic acid is heterologous to that transgenic organism or transgenic cell.

A “transgene” refers to a gene such as an oxidase enzyme or rubredoxin or rubredoxin reductase gene that has been introduced into the genome by transformation and preferably is stably maintained. Preferably, transgenes include genes that are heterologous to the genes of a particular cell or organism to be transformed. Additionally, transgenes may comprise native genes inserted into a non-native organism, or chimeric genes. The term “endogenous gene” refers to a native gene in its natural location in the genome of an organism. A “foreign” gene refers to a gene not normally found in the host organism but that is introduced by gene transfer.

Methods for the production of transgenic non-human organisms are well known in the art; see, e.g. Lee-Yoon Low et al., Transgenic Plants: Gene constructs, vector and transformation method. 2018. DOI.10.5772/intechopen.79369; Pinkert, C. A. (ed.) 1994. Transgenic animal technology: A laboratory handbook. Academic Press, Inc., San Diedo, Calif.; Monastersky G. M. and Robl, J. M. (ed.) (1995) Strategies in Transgenic Animal Science. ASM Press. Washington D.C); Sambrook, loc.cit, Ausubel, loc.cit).

Another aspect of the invention is directed to a composition produced by the method of or by the non-human host cell, wherein the composition comprises:

the oxidase enzyme as defined herein; and
the at least one oxidised terpene product selected from myrcene aldehyde, alpha sinensal, beta sinensal, trans alpha santalol, trans beta santalol, lanceol-aldehyde, nootkatone, vetivone, rotundone, rebaudiosides, 8-hydroxygeraniol, 8-hydroxynerol, 9,10-epoxygeranylacetone, hexadecenal, farnesol, denderalasin bicyclo-octanediol, oxidized alpha guaiene or oxidized beta guaiene, or combination thereof; and
optionally, one or more terpene substrates.

Another aspect of the invention is directed to a composition produced by the methods as disclosed hereinabove.

Preferably, the composition includes at least one oxidised terpene product. The at least one oxidised terpene product includes alpha sinensal, beta sinensal, trans alpha santalol and trans beta santalol, lanceol-aldehyde, nootkatone, vetivone, rotundone, rebaudiosides, 8-hydroxygeraniol, 8-hydroxynerol, or 9,10-epoxygeranylacetone.

Preferably the oxidised terpene product is selected from alpha sinensal, beta sinensal, trans alpha santalol, trans beta santalol, nootkatone and farnesol.

Another aspect of the invention includes a fermentation composition comprising:
the non-human host cell as defined above, cultured in a culture medium; and
the at least one oxidised terpene product produced from the non-human host cell as a major compound, wherein the fermentation composition includes the terpene substrate and optionally one or more co-products are minor compounds.

The term “fermentation” is used to refer to culturing microorganisms that utilize carbon sources, such as sugar, as an energy source to produce a desired product.

The term “culture medium” refers to a medium which allows growth of biomass and production of microbial metabolites. It contains a source of carbon and may further contain a source of nitrogen, a source of phosphorus, a source of vitamins, a source of minerals, and the like.

As used herein, the term “fermentation medium” may be used synonymously with “culture medium.” Generally, the term “fermentation medium” may be used to refer to a medium which is suitable for culturing microorganisms for a prolonged time period to produce a desired compound from microorganisms.

The term “medium” refers to a culture medium and/or fermentation medium. The “medium” can be liquid or semi-solid. A given medium may be both a culture medium and a fermentation medium.

The term “whole cell broth” refers to the entire contents of a vessel (e.g., a flask, plate, fermentor and the like), including cells, aqueous phase, compounds produced in hydrocarbon phase and/or emulsion. Thus, the whole cell broth includes the mixture of a culture medium comprising water, carbon source (e.g., sugar), minerals, vitamins, other dissolved or suspended materials, microorganisms, metabolites and compounds produced by microorganisms, and all other constituents of the material held in the vessel in which one or more terpene substrates and/or oxidised terpene products including for example oxidised sesquiterpene products are being made by the microorganisms.

The term “fermentation composition” is used interchangeably with “whole cell broth.” The fermentation composition can also include an overlay if it is added to the vessel during fermentation.

The fermentation process may be carried out in two stages-a build stage and a production stage. The build stage is carried out for a period of time sufficient to produce an amount of cellular biomass that can support production of the terpene substrate and consequently the oxidised terpene product during the production stage. The build stage is carried out for a period of time sufficient for the population present at the time of inoculation to undergo a plurality of doublings until a desired cell density is reached.

The method of producing the terpene substrate and consequently the oxidised terpene product may comprise conducting fermentation of the genetically modified host cell typically under aerobic conditions sufficient to allow growth and maintenance of the genetically modified host cell; then subsequently providing microaerobic fermentation conditions sufficient to induce production of the terpene substrate (like myrcene or other terpenes, co-products), and maintaining the microaerobic conditions throughout the fermentation run. The microaerobic conditions may be used throughout the fermentation run. An inducing agent may be added during the production stage to activate a promoter or to relieve repression of a transcriptional regulator to promote production of terpene substrates and/or oxidised terpene products.

The method of producing the terpene substrate as well as the alkane oxidation system and consequently the oxidised terpene product may comprise culturing the at least one microbial host cells in separate build and production culture media. For example, the method can comprise culturing the at least one genetically modified microbial host cell in a build stage wherein the cell is cultured under non-producing conditions (e.g., non-inducing conditions) to produce an inoculum, then transferring the inoculum into a second fermentation medium under conditions suitable to induce the terpene substrate production (e.g., inducing conditions), and maintaining steady state conditions in the second fermentation stage to produce a cell culture containing the terpene substrate as well as the alkane oxidation system and consequently the oxidised terpene product.

In another embodiment, the terpene substrate is produced by one host cell while another host cell provides the artificial alkane oxidase system.

In a one embodiment, the terpene substrate produced by one host cell and the artificial alkane oxidase system provided by another host cell belong to same species, for example the two host cells are from Rhodobacter species.

In another embodiment, the two host cells are provided in a mixed fermentation process.

During the fermentation process, the artificial alkane oxidase system in the host cell, provides the oxidase enzyme, the terpene synthase if needed, optionally the electron transfer compound and the further optionally the electron transfer compound regeneration enzyme. The allylic oxidation of the at least one terpene substrate produces the oxidised terpene product.

Culture media and culture conditions for the maintenance and growth of microbial cultures are well known to those skilled in the art of microbiology or fermentation science (see, for example, Bailey et al., Biochemical Engineering Fundamentals, second edition, McGraw Hill, New York, 1986). Appropriate culture medium, pH, temperature, and requirements for aerobic, microaerobic, or anaerobic conditions may be selected depending on the specific requirements of the microbial host cell, the fermentation, and the process.

The culture medium for use in the methods of producing oxidised terpene products as provided herein may include any culture medium in which a genetically modified microorganism capable of producing terpenes can subsist, i.e., support and maintain growth and viability. The culture medium may also promote the biosynthetic pathway necessary to produce the desired terpene substrate and consequently the oxidised terpene product.

The culture medium may be an aqueous solution comprising assimilable carbon, nitrogen and phosphate sources. Such a medium can also include appropriate salts, minerals, metals and other nutrients. The carbon source and each of the essential cell nutrients may be added incrementally or continuously to the fermentation media, and each required nutrient is maintained at essentially the minimum level needed for efficient assimilation by growing cells, for example, in accordance with a predetermined cell growth curve based on the metabolic or respiratory function of the cells which convert the carbon source to a biomass.

The carbon source may be a monosaccharide (simple sugar), a disaccharide, a polysaccharide, a non-fermentable carbon source, or one or more combinations thereof. Non-limiting examples of suitable monosaccharides include glucose, galactose, mannose, fructose, ribose, and combinations thereof. Non-limiting examples of suitable disaccharides include sucrose, lactose, maltose, trehalose, cellobiose, and combinations thereof. Non-limiting examples of suitable polysaccharides include starch, glycogen, cellulose, chitin, and combinations thereof. Non-limiting examples of suitable non-fermentable carbon sources include acetate and glycerol. The carbon source may be derived from a wide variety of crops and sources. Some non-limiting examples of suitable crops or sources include sugar cane, bagasse, miscanthus, sugar beet, sorghum, grain sorghum, switchgrass, barley, hemp, kenaf, potatoes, sweet potatoes, cassava, sunflower, fruit, molasses, whey or skim milk, corn, stover, grain, wheat, wood, paper, straw, cotton, many types of cellulose waste, and other biomass. The suitable crops or sources may include sugar cane, sugar beet and corn. The sugar source may be cane juice or molasses. Any combination of the above carbon sources may be used.

The suitable medium may be supplemented with one or more additional agents, such as, for example, an inducer (e.g., when one or more nucleotide sequences encoding a gene product are under the control of an inducible promoter), a repressor (e.g., when one or more nucleotide sequences encoding a gene product are under the control of a repressible promoter), or a selection agent (e.g., an antibiotic to select for microorganisms comprising the genetic modifications).

A liquid organic overlay may be added to the culture medium during the production stage of the fermentation. A liquid organic overlay may be an immiscible organic liquid which is in contact with the aqueous culture medium, and terpene substrate and other co-products and the oxidised terpene product secreted from microorganisms can be captured in the liquid organic overlay. A liquid organic overlay can reduce evaporation of volatile monoterpenes from the fermentation vessel as well reduce potential terpene substrate and/or the oxidised terpene product toxicity to micro-organism. Examples of an overlay include, but are not limited to, isopropyl myristate (IPM) or other hydrocarbon liquids such as white mineral oils or polyalphaolefins.

The fermentation methods may be performed in a suitable container or vessel, including but not limited to, a cell culture plate, a flask, or a fermentor. The fermentation may be conducted in a closed system to trap terpenes in the gas phase. For example, the closed system may include a series of vessels connected to one another to trap offgas including monoterpenes in the vapor phase. For example, a first vessel may contain a culture medium comprising an aqueous medium and genetically modified microorganisms. A second vessel comprising an organic overlay may be connected in series with the first vessel to trap the volatile terpenes. One or more additional vessels may be connected to the first vessel in series and/or parallel to capture a gaseous composition comprising the terpene substrate and other terpene co-products and the oxidised terpene product. Furthermore, the methods can be performed at any scale of fermentation known in the art to support industrial production of microbial products. Any suitable fermentor may be used including a stirred tank fermentor, an airlift fermentor, a bubble fermentor, or any combination thereof.

Further, the methods can be performed at any volume of fermentation, e.g., from lab scale (e.g., 10 ml to 20 L) to pilot scale (e.g., 20L to 500 L) to industrial scale (e.g., 500 L to ≥500,000 L) fermentations.

Disclosed herein are fermentation compositions comprising a genetically modified microbial host cell described herein, a culture medium, and terpene substrate and the oxidised terpene product produced from the genetically modified microbial host cell. In the fermentation compositions provided herein, the oxidised terpene product is typically a major compound and comprised are spare terpene substrate as well as the one or more coproducts (which are concurrently produced with terpene) as minor compounds.

In an embodiment, the fermentation compositions comprise with respect to the terpenes at least about 50% major compound and less than about 50% minor compounds, compared to the total amount of terpene substrate, based on relative area % of terpene peaks shown in a GC chromatogram of the monoterpenes.

In another embodiments, the fermentation composition comprises with respect to the terpenes at least 55% or 60% or 65% or 70% or 75% or 80% or 85% or 90% or 95% of the major compound, compared to the total amount of terpene substrate in the culture medium.

In another embodiments, the fermentation composition comprises with respect to the terpenes at least 45% or 40% or 35% or 30% or 25% or 20% or 15% or 10% or 5% of the minor compound, compared to the total amount of terpene substrate in the culture medium.

In another embodiment, the major compound includes at least two oxidised terpene products. In a further embodiment, the at least two oxidised terpene products include trans alpha santalol and trans beta santalol.

In another embodiment, with use of a terpene synthase producing more than one terpene, for example a mixture of alpha guaiene and beta guaiene, the at least two oxidised terpene products are as well a mixture of oxidised terpene products, for example oxidised alpha guaiene and oxidised beta guaiene, formed as major compound.

Another aspect of the present invention is directed to a method of fermentative production of at least one oxidised terpene product, the method comprising:

providing a non-human host cell with the oxidase enzyme system or the expression cassette as described herein,
optionally providing at least one alkane synthase to the host cell for alkane substrate production;
culturing the non-human host cell in a culture medium under conditions for the host cell to produce the encoded polypeptides in active form;
optionally, producing one or more alkane substrates and/or providing one or more alkane substrates to the host cell; and
producing at least one oxidised alkane product; and
optionally, purifying the at least one oxidised alkane product.

In an embodiment, the method of fermentative production of at least one oxidised terpene product includes providing a bacterial host cell, preferably a Rhodobacter strain.

In another embodiment, the method of fermentative production of at least one oxidised terpene product includes production of sinensal.

In another embodiment, the method of fermentative production of at least one oxidised terpene product includes the oxidase enzyme system comprising
a. an oxidase enzyme; and
b. one or more enzymes to provide one or more alkanes and optionally,
c. an electron transfer compound suitable to transfer at least one electron to the oxidase enzyme; and further optionally
d. an electron transfer compound regeneration enzyme suitable to reduce the electron transfer compound of c. when it is in its oxidized state;
   wherein the oxidase enzyme is an amino acid sequence with a sequence identity of at least 62%, 66%, 69%, 70%, 75%, 80%, 85%, 90%, 94%, 95%, 98% or 99% with any one of SEQ ID NO: 1, 10, 11, 23 to 43 or a fragment thereof or a variant thereof.

Another aspect of the present invention is directed to use of the alkane oxidation system as disclosed; or expression of nucleic acid sequence wherein the synthetic nucleic acid sequence comprises a nucleic acid sequence encoding the oxidase enzyme, and optionally the rubredoxin peptide and/or the rubredoxin reductase peptide or expression cassette thereof; or expression cassette of the alkane oxidation system for oxidation of the terpene substrate and the production of one or more oxidised terpene products.

Another aspect of the invention is directed to a method of producing alkane oxidised product or a composition wherein the oxidized alkane product is selected from an alcohol or an aldehyde, or both, including myrcene aldehyde, alpha sinensal, beta sinensal, trans alpha santalol and trans beta santalol, lanceol-aldehyde, nootkatone, vetivone, rotundone, rebaudiosides, 8-hydroxygeraniol, 8-hydroxynerol, 9,10-epoxygeranylacetone, hexadecenal, farnesol, denderalasin and bicyclo-octanediol, oxidized alpha guaiene, oxidized beta guaiene, or a combination thereof. Preferably the oxidised product is selected from alpha sinensal, beta sinensal, trans alpha santalol, trans beta santalol, nootkatone and farnesol.

Another aspect of the present invention is directed to a composition produced by the alkane oxidation system of the invention, the methods of the invention, or the non-human host cell of the invention, wherein the composition comprises one or more oxidised terpene products, for example but not limited to alpha sinensal, beta sinensal, trans alpha santalol and trans beta santalol, lanceol-aldehyde, nootkatone, vetivone, rotundone, rebaudiosides, 8-hydroxygeraniol, 8-hydroxynerol, or 9,10-epoxygeranylacetone, or combination thereof, and optionally one or more terpene substrates, preferably in a weight ratio of 3:1 to 1:10,000 to their corresponding oxidised terpene product, and optionally the oxidase enzyme as defined hereinabove. In one aspect of the invention, the compositions of the invention also comprise the one or more electron transfer compounds, including preferably rubredoxin, and optionally at least one electron transfer compound regeneration enzyme suitable to reduce the electron transfer compound when it is in its oxidized state, preferably rubredoxin reductase.

In one embodiment the weight ratio of terpene substrate to the oxidised terpene product(s) is 250 to 1, or 220 to 1, or 200 to 1, or 180 to 1, 170 to 1, 160 to 1. In a preferred embodiment, the weight ratio is 150 to 1, 130 to 1, or 120 to 1, 110 to 1, 100 to 1. In a more preferred embodiment, the weight ratio is of 99 or less to 1, but higher than 0.1:1; for example the weight ratio of farnesene to oxidised terpene product is from 99:1 to 10:1, preferably 31 or less to 1, for example 20, 18, 17 or 14 to 1.

In an embodiment, at least 0.3% (w/w) of the oxidised terpene product(s) is formed. In a preferred embodiment 0.6% (w/w) sinensal is formed as the oxidised terpene product.

In case more than one oxidised terpene product is produced for example by providing both alpha- and beta farnesene the ratio of the first oxidised terpen product to the second oxidised terpen product is in one embodiment from 0.8: to 1 to 1:0.8, preferably 0.9: to 1 to 1:0.9, more preferably 1:1.

Another aspect of the present invention related to a method for preparing a variant polypeptide having the oxidase enzyme activity, the method comprising steps of:
a) selecting a synthetic nucleic acid sequence as disclosed here in or a nucleic acid encoding an amino acid sequence with a sequence identity of at least 62%, 66%, 69%, 70%, 75%, 80%, 85%, 90%, 94%, 95%, 98% or 99% with any one of SEQ ID NO: 1, 10, 11, 23 to 43 or a fragment thereof, preferably 1, 23, 24, 43;
b) modifying the selected nucleic acid to obtain at least one mutant nucleic acid;
c) transforming the non-human host cells with the mutant nucleic acid sequence to express the oxidase enzyme encoded by the mutant nucleic acid sequence;
d) screening the oxidase enzyme for at least one modified property in addition to the functionality of the oxidase enzyme encoded by the selected nucleic acid of a); and,
e) optionally, repeating the process steps (a) to (d) to obtain an oxidase enzyme with a desired variant functionality;
f) optionally, isolating the mutant nucleic acid sequence from the transformed non-human host cell obtained in step (c).

The functionality may be an oxidase enzyme of SEQ ID NO: 1, 10, 11, 23, 24, or any of SEQ ID NO: 25 to 43, preferably SEQ ID NO: 1, 10, 11 or 25 to 43.

A “polypeptide variant”/“variant polypeptide” as referred to herein means a polypeptide having an oxidase enzyme activity and being substantially homologous to the polypeptide according to any of the above embodiments for oxidase enzyme, but having an amino acid sequence different from that encoded by any of the nucleic acid sequences of the invention because of one or more deletions, insertions or substitutions.

Variants can comprise conservatively substituted sequences, meaning that a given amino acid residue is replaced by a residue having similar physiochemical characteristics. Examples of conservative substitutions include substitution of one aliphatic residue for another, such as He, Val, Leu, or Ala for one another, or substitutions of one polar residue for another, such as between Lys and Arg; Glu and Asp; or Gin and Asn. See Zubay, Biochemistry, 1983, Addison-Wesley Pub. Co. The effects of such substitutions can be calculated using substitution score matrices such a PAM-120, PAM-200, and PAM-250 as discussed in Altschul, J. Mol. Biol., 1991, 219, 555-565. Other such conservative substitutions, for example substitutions of entire regions having similar hydrophobicity characteristics, are well known. Naturally occurring peptide variants are also encompassed by the invention. Examples of such variants are proteins that result from alternate mRNA splicing events or from proteolytic cleavage of the polypeptides described herein. Variations attributable to proteolysis include, for example, differences in the N- or C-termini upon expression in different types of host cells, due to proteolytic removal of one or more terminal amino acids from the polypeptides encoded by the sequences of the invention.

Table: Variant below provides for conserved amino acid positions with allowed exchanges with reference to the SEQ ID NO: 1.

TABLE
VARIANTS

| Position (s) corresponding to | Amino acid |
|---|---|
| the one in SEQ ID NO: 1 | (one letter code) |
| 70, 102, 253 | A |
| 74, 152, 223, 230, 239, 267, | A or G, preferably A |
| 292, 296, 297 | |
| 66 | C |
| 69, 295 | D or E, preferably D |
| 210, 231, 263, 268 | E or D, preferably E |
| 29, 200 | F |
| 93 | F or W or S, preferably S |
| | or F, more preferably S |
| 255 | F or Y, preferably F |
| 33, 180 | G |
| 36, 73, 229, 293, | G or A, preferably G |
| 68, 72, 10, 165, 209, 244, 247, | H |
| 248, 266, 269, 288, 291 | |
| 260 | K or H or R, preferably K |
| 54, 55, 190, 259, 276 | I or L, preferably I |
| 14, 35, 37, 186, 188, 191, 207, | L or I, preferably L |
| 233, 237, 238, 245, 249, 261 | |
| 234 | L or I or V, preferably L |
| 275 | L or I or V, preferably L |
| | or I, more preferably L |
| 67, 264 | L or I or V, preferably L |
| | or V, more preferably L |
| 246, 258 | M |
| 187 | M or R or K, preferably M |
| 137, 225, 242, 257 | N or Q, preferably N |
| 6, 18, 23, 151, 160, 192, 208, 211, | P |
| 228, 236, 251, 254, 274, 284 | |
| 241 | Q or N, preferably Q |
| 103 | R |
| 161, 265, 294 | R or K or H, preferably R |
| 71, 76, 224, 273 | S or T, preferably S |
| 3, 34, 193, 235, 252 | T or S, preferably T |
| 75, 168 | V or L, preferably V |
| 262, 232 | W or F or Y, preferably W |
| 189 | W or S or T, preferably S |
| 240, 243, 250 | Y |
| 256 | Y or F or W, preferably F |
| | or Y, more preferably Y |

Variants of the polypeptides of the invention may be used to attain for example desired enhanced or reduced enzymatic activity, modified regiochemistry or stereochemistry, or altered substrate utilization or product distribution, increased affinity for the substrate, improved specificity for the production of one or more desired compounds, increased velocity of the enzyme reaction, higher activity or stability in a specific environment (pH, temperature, solvent, etc), or improved expression level in a desired expression system. A variant or site directed mutant may be made by any method known in the art. Variants and derivatives of native polypeptides can be obtained by isolating naturally-occurring variants, or the nucleotide sequence of variants, of other or same plant lines or species, or by artificially programming mutations of nucleotide sequences coding for the polypeptides of the invention. Alterations of the native amino acid sequence can be accomplished by any of a number of conventional methods.

Polypeptide variants resulting from a fusion of additional peptide sequences at the amino and carboxyl terminal ends of the polypeptides of the invention can be used to enhance expression of the polypeptides, be useful in the purification of the protein or improve the enzymatic activity of the polypeptide in a desired environment or expression system. Such additional peptide sequences may be signal peptides, for example. Accordingly, the present invention encompasses variants of the polypeptides of the invention, such as those obtained by fusion with other oligo- or polypeptides and/or those which are linked to signal peptides. Fusion polypeptides encompassed by the invention also comprise fusion polypeptides resulting from a fusion of other functional proteins, such as other proteins from the terpene biosynthesis pathway.

Therefore, in an embodiment, the present invention provides a method for preparing a variant polypeptide having an oxidase enzyme activity, as described in any of the above embodiments, and comprising the steps of:
(a) selecting a synthetic nucleic acid sequence as disclosed herein or a nucleic acid encoding an amino acid sequence with a sequence identity of at least 62%, 66%, 69%, 70%, 75%, 80%, 85%, 90%, 94%, 95%, 98% or 99% with any one of SEQ ID NO: 1, 10, 11, 23 to 43 or a fragment thereof;
(b) modifying the selected nucleic acid to obtain at least one mutant nucleic acid;
(c) transforming host cells or unicellular organisms with the mutant nucleic acid sequence to express a polypeptide encoded by the mutant nucleic acid sequence;
(d) screening the polypeptide for at least one modified property; and,
(e) optionally, if the polypeptide has no desired variant oxidase enzyme activity, repeating the process steps (a) to (d) until a polypeptide with a desired variant oxidase enzyme activity is obtained;
(f) optionally, if a polypeptide having a desired variant oxidase enzyme activity was identified in step (d), isolating the corresponding mutant nucleic acid obtained in step (c).

According to a preferred embodiment, the variant polypeptide prepared is capable of producing the oxidised terpene product as a major compound.

According to an even more preferred embodiment, it is capable of producing a mixture of a major compound and a minor compound. The oxidised terpene product is the major compound and one or more co-products are minor compounds, wherein oxidised terpene product represents at least 60%, preferably at least 80%, preferably at least 90% of the mixture.

In step (b), a large number of mutant nucleic acid sequences may be created, for example by random mutagenesis, site-specific mutagenesis, or DNA shuffling. The detailed procedures of gene shuffling are found in Stemmer, DNA shuffling by random fragmentation and reassembly: in vitro recombination for molecular evolution. Proc Natl Acad Sci USA., 1994, 91(22): 10747-1075. In short, DNA shuffling refers to a process of random recombination of known sequences in vitro, involving at least two nucleic acids selected for recombination. For example mutations can be introduced at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered gene wherein predetermined codons can be altered by substitution, deletion or insertion.

Accordingly, the polypeptide comprising SEQ ID NO: 1 may be recombined with any other oxidase enzyme encoding nucleic acids, for example isolated from an organism other than Pseudomonas. sp. Thus, mutant nucleic acids may be obtained and separated, which may be used for transforming a host cell according to standard procedures, for example such as disclosed in the present examples.

In step (d), the polypeptide obtained in step (c) is screened for at least one modified property, for example a desired modified enzymatic activity. Examples of desired enzymatic activities, for which an expressed polypeptide may be screened, include enhanced or reduced enzymatic activity, as measured by KM or Vma χ value, modified regiochemistry or stereochemistry and altered substrate utilization or product distribution.

The screening of enzymatic activity can be performed according to procedures familiar to the skilled person and those disclosed in the present examples. Step (e) provides for repetition of process steps (a)-(d), which may preferably be performed in parallel. Accordingly, by creating a significant number of mutant nucleic acids, many host cells may be transformed with different mutant nucleic acids at the same time, allowing for the subsequent screening of an elevated number of polypeptides. The chances of obtaining a desired variant polypeptide may thus be increased at the discretion of the skilled person.

### Advantages:

Production of oxidised terpene products like sinensals from a microbial culture from glucose is challenging, and hitherto no enzymes which mediate oxidation on diallylic positions on terpenes, preferably sesquiterpenes are known. The method disclosed is applicable to a variety of terpenes substrates with diallylic groups such as Santalene, valencene, etc.

### METHOD A

### Gas Chromatography and Mass Spectrum (GC & MS)

The culture is extracted with 1 ml dichloromethane (DCM). The DCM phase is collected, dried over anhydrous Na2SO4 and injected into a 7890A gas chromatograph (Agilent) equipped with a mass selective detector (Model 5975C, Agilent), scanning in the range 45-450 m/z. Splitless injection of 1 μl sample was performed at 250° C. on a Zebron ZB-MS column (30 m×0.25 mm, 0.25 μm thickness; Phenomenex) at a helium flow rate of 1 ml/min. The temperature programme was 2.25 min at 45° C., then at 40° C./min to 300° C., then 3 min at 300° C. The MS detector was switched off at room temperature for 8 min to prevent saturation, in the case of myrcene conversion experiments, and for 14 min in the case of farnesene experiments. Compounds were identified by comparing their retention index and mass spectra to the NIST 8 database or literature data.

The present invention is illustrated in more detail by the following embodiments and combinations of embodiments which result from the corresponding dependency references and links:
I. An artificial alkane oxidation system comprising
   a. an oxidase enzyme, and
   b. one or more enzymes providing at least one alkane, preferably a terpene synthase protein;
   optionally with
   c. an electron transfer compound suitable to transfer at least one electron to the oxidase enzyme and further optionally
   d. an electron transfer compound regeneration enzyme suitable to reduce the electron transfer compound of c. when it is in its oxidized state
   wherein the oxidase enzyme is with an amino acid sequence with a sequence identity of at least 62%, 66%, 69%, 70%, 75%, 80%, 85%, 90%, 94%, 95%, 98% or 99% with Any one of SEQ ID NO: 1, 10, 11, 23 to 43 or a fragment thereof or a variant thereof.
II. The artificial alkane oxidation system of embodiment I wherein the alkane is made of five carbon building blocks with one to five such buildings blocks; preferably a terpene and wherein at least one alkane is a terpene, and at least one terpene synthase protein as component b.
III. The artificial alkane oxidation system of embodiment I or II, wherein the electron transfer compound is an electron transfer protein, preferably of the F1-S0 type, more preferable a rubredoxin peptide with an amino acid sequence with a sequence identity of at least 62%, 66%, 69%, 70%, 75%, 80%, 85%, 90%, 94%, 95%, 98% or 99% with any one of SEQ ID NO: 2 or 45 or a fragment thereof or a variant thereof, or/and the electron transfer compound regeneration enzyme is an electron transfer protein reductase, preferably rubredoxin reductase, more preferably a rubredoxin reductase peptide with a sequence identity of at least 62%, 66%, 69%, 70%, 75%, 80%, 85%, 90%, 94%, 95%, 98% or 99% with SEQ ID NO: 3 or fragment thereof.
IV. A synthetic nucleic acid sequence with a sequence identity of at least 62%, 66%, 69%, 70%, 75%, 80%, 85%, 90%, 94%, 95%, 98% or 99% with any one of SEQ ID NO:4, SEQ ID NO:12, SEQ ID NO:17, SEQ ID NO:18, SEQ ID NO: 19, or SEQ ID NO: 44, wherein the synthetic nucleic acid sequence comprises a nucleic acid sequence encoding the oxidase enzyme as defined in embodiment I, and optionally the rubredoxin peptide and/or the rubredoxin reductase peptide of embodiment II.
V. An expression cassette comprising the synthetic nucleic acid sequence of embodiment IV.
VI. An expression cassette comprising the nucleic acid sequence encoding the alkane oxidation system of embodiment I, II or III.
VII. An expression cassette comprising the nucleic acid sequence of the alkane oxidation system, the nucleic acid sequences comprising:
   a. a nucleic acid sequence for an oxidase enzyme with a sequence identity of at least 62%, 66%, 69%, 70%, 75%, 80%, 85%, 90%, 94%, 95%, 98% or 99% with any one of SEQ ID NO:20, or encoding for an oxidase enzyme with a sequence identity of at least 62%, 66%, 69%, 70%, 75%, 80%, 85%, 90%, 94%, 95%, 98% or 99% with any one of SEQ ID NO: 1, 10, 11, 23 to 43 and
   b. a nucleic acid sequence for a rubredoxin peptide with a sequence identity of at least 62%, 66%, 69%, 70%, 75%, 80%, 85%, 90%, 94%, 95%, 98% or 99% with of any one of SEQ ID NO:21, and optionally
   c. a nucleic acid sequence for a rubredoxin reductase peptide with a sequence identity of at least 62%, 66%, 69%, 70%, 75%, 80%, 85%, 90%, 94%, 95%, 98% or 99% with of SEQ ID NO:22 or combination of b and c with a.
VIII. A method of oxidation of at least one terpene substrate, the method comprising:
   a. providing
      i. the artificial alkane oxidase system of any of embodiment I to III, or
      ii. expression of the synthetic nucleic acid sequence of embodiment IV, or expression cassette of embodiment V to VII or
      iii. expression of a nucleic acid sequence encoding the oxidase enzyme with an amino acid sequence with a sequence identity of at least 62%, 66%, 69%, 70%, 75%, 80%, 85%, 90%, 94%, 95%, 98% or 99% with Any one of SEQ ID NO: 1, 10, 11, 23 to 43 or a fragment thereof or a variant thereof, and
      iv. optionally the at least one terpene substrate if not produced by the alkane oxidation system;
   for oxidation of the at least one terpene substrate,
   b. allylic oxidation of the terpene substrate to produce at least one oxidised terpene product
   c. extracting the at least one oxidised terpene product.
IX. The method of embodiment VIII, wherein the terpene substrate includes diterpenes, monoterpenes or sesquiterpenes, for example but not limited to alpha farnesene, beta farnesene, alpha bisabolene, beta bisabolene, alpha bergamotene, beta bergamotene, alpha santalene, beta santalene, valencene, alpha guaiene, monoterpene geraniol, nerol monoterpenes, geranylactone or combination thereof.
X. The method of embodiment VIII or IX, wherein the oxidised terpene product is an alcohol or an aldehyde, or both.
XI. The method of any one of embodiment VIII to X, wherein the at least one oxidised terpene product is selected from selected from myrcene aldehyde, alpha sinensal, beta sinensal, trans alpha santalol and trans beta santalol, lanceol-aldehyde, nootkatone, vetivone, rotundone, rebaudiosides, 8-hydroxygeraniol, 8-hydroxynerol, 9,10-epoxygeranylacetone. hexadecenal, farnesol, denderalasin and bicyclo-octanediol, oxidized alpha guaiene, oxidized beta guaiene, or combination thereof.
XII. A non-human host cell comprising the artificial alkane oxidation system of any of embodiment I to III, the synthetic nucleic acid of embodiment IV, the expression cassettes of embodiment V to VII or suitable for oxidation of the terpene substrate by the methods according to embodiment VIII to XI.
XIII. The non-human host cell of embodiment XII, wherein the host cell is a Rhodobacter species
XIV. A composition produced by the method of embodiment VIII to XI or the non-human host cell of embodiment XII or XIII, wherein the composition comprises alpha sinensal, beta sinensal, trans alpha santalol and trans beta santalol, lanceol-aldehyde, nootkatone, vetivone, rotundone, rebaudiosides, 8-hydroxygeraniol, 8-hydroxynerol, or 9,10-epoxygeranylacetone, or combination thereof, and optionally one or more terpene substrates and optionally the oxidase enzyme as defined in any of the preceding embodiments.
XV. A fermentation composition comprising:
   the non-human host cell of embodiment XII or XIII cultured in a culture medium; and
   the at least one oxidised terpene product produced from the non-human host cell as a major compound,
   wherein the fermentation composition includes the terpene substrate and optionally one or more co-products are minor compounds.
XVI. Use of the alkane oxidation system of embodiment I to III, or expression of nucleic acid sequence of embodiment IV, or expression cassette of embodiment V to VII, the non-human host cells of embodiment XII or the fermentation composition of embodiment XV for oxidation of the terpene substrate.
XVII. The alkane oxidation system of embodiment I to III, wherein the oxidase enzyme is encoded by SEQ ID NO:20, rubredoxin peptide is encoded by SEQ ID NO:21, and/or the rubredoxin reductase peptide is encoded by SEQ ID NO:22.
XVIII. An alkane oxidation system comprising
   a. an oxidase enzyme with an amino acid sequence with a sequence identity of at least 62%, 66%, 69%, 70%, 75%, 80%, 85%, 90%, 94%, 95%, 98% or 99% with Any one of SEQ ID NO: 1, 10, 11, 23 to 43 or a fragment thereof or a variant thereof, and
   b. a heterologus sesquiterpene synthase protein, optionally with
   c. a rubredoxin peptide with an amino acid sequence with a sequence identity of at least 62%, 66%, 69%, 70%, 75%, 80%, 85%, 90%, 94%, 95%, 98% or 99% with of any one of SEQ ID NO:2 or a fragment thereof, or/and
   d. a rubredoxin reductase peptide with a sequence identity of at least 62%, 66%, 69%, 70%, 75%, 80%, 85%, 90%, 94%, 95%, 98% or 99% with of SEQ ID NO:3 or fragment thereof, or combination of b and c with a.
XIX. An expression cassette comprising the nucleic acid sequence encoding the alkane oxidation system of embodiment XVII and XVIII.
XX. A method of oxidation of at least one terpene substrate, preferably sesquiterpene substrate, the method comprising:
   a. providing
      i. the alkane oxidation system of embodiment I or II or XVII or XVIII or
      ii. expression of nucleic acid sequence of embodiment IV, or expression cassette of embodiment V or VII or
      iii. expression of nucleic acid sequence encoding the oxidase enzyme with an amino acid sequence with a sequence identity of at least 70% Any one of SEQ ID NO: 1, 10, 11, 23 to 43 (alkB) or a fragment thereof or a variant thereof, and
      iv. optionally a terpene, preferably sesquiterpene substrate;
   for hydroxylation of the terpene, preferably sesquiterpene substrate,
   b. allylic oxidation of the terpene, preferably sesquiterpene substrate with the alkane oxidation system to produce one or more oxidised terpene product
   c. extracting the one or more oxidised terpene product.
XXI. A method of producing at least one oxidised terpene product by the method as described in any one of embodiment VIII to XII.
XXII. Use of the non-human host cells of embodiment XII or the fermentation composition of embodiment XV for oxidised terpene products.
XXIII. A method of fermentative production of at least one oxidised terpene product, the method comprising:
   providing a non-human host cell with the oxidase enzyme system as described in any one of the previous embodiments;
   culturing the non-human host cell in the culture medium to produce at least one oxidised terpene product.
XXIV. A kit for the artificial alkane oxidation system, the kit comprising
   a first isolated nucleic acid sequence with a sequence identity of at least 62%, 66%, 69%, 70%, 75%, 80%, 85%, 90%, 94%, 95%, 98% or 99% with any one of SEQ ID NO: 1, 10, 11, 23 to 43 wherein the first nucleic acid sequence comprises a nucleic acid sequence encoding the oxidase enzyme as defined in embodiment I, and
   a second isolated nucleic acid sequence, not physically connected to the first nucleic acid encoding an electron transfer protein and
   optionally a third nucleic acid sequence encoding an electron transfer protein reductase, wherein the third nucleic acid sequence may be fused to the first and/or second nucleic acid sequence.
XXV. A method for preparing a variant polypeptide having a functionality as required for their use as any components a to d, comprising the steps of:
   a) selecting a nucleic acid of the invention or a nucleic acid encoding a polypeptide of the invention;
   b) modifying the selected nucleic acid to obtain at least one mutant nucleic acid;
   c) transforming host cells or unicellular organisms with the mutant nucleic acid sequence to express a polypeptide encoded by the mutant nucleic acid sequence;
   d) screening the polypeptide for at least one modified property in addition to the functionality of the polypeptide encoded by the selected nucleic acid of a); and,
   e) optionally, if the polypeptide has no desired functionality of the polypeptide, repeating the process steps (a) to (d) until a polypeptide with a desired variant functionality as any component a to d is obtained;
   f) optionally, if a polypeptide having a desired variant functionality was identified in step (d), isolating the corresponding mutant nucleic acid obtained in step (c).

### EXAMPLES

The present invention is further illustrated in combination with the following examples. These examples are provided to exemplify the present invention but are not intended to restrict the scope of the presently claimed invention in any way. The terms and abbreviations in the examples have their common meanings. For example, “%”, “Eq. wt.”, “Eq.”, “C”, “wt. %”, “% w/w”, “% w/v” and “gm” represent “percentage”, “Equivalent Weight”, “Equivalents”, “degree Celsius”, “percent by weight”, “percent weight by weight”, “percent weight by volume” and “gram” respectively.

This invention involves digital sequence information of biological material published by third parties. For example, for the sequences of SEQ ID NO: 1 to 3 and 20 to 21 the biological material as the source of the published digital sequence information was disclosed to be sourced from The Netherlands. (Soares-Castro 2017 Appl Environ Microbiol 83:e03112-16.).

### Sequences

| SEQ ID NO: | Details of Sequence | Length |
|---|---|---|
| 1 | M1-alkB - Amino acid Sequence \| alkane 1-mono- | 297 |
| | oxygenase AlkB | |
| 2 | M1-rubredoxin - Amino acid Sequence \| rubredoxin peptide | 52 |
| 3 | M1-rubredoxin reductase - Amino acid Sequence \| | 384 |
| | rubredoxin reductase | |
| 4 | Synthetic construct - M1-alkB-rubredoxin-rubredoxin | 2285 |
| | reductase - Nucleic acid Sequence \| alkane oxidation | |
| | system components a & c & d | |
| 5 | AaBFS - Nucleic acid Sequence \| β-farnesene synthase | 2135 |
| 6 | AaBFS - Amino acid Sequence \| β-farnesene synthase | 574 |
| 7 | MdAFS - Amino acid Sequence \| α-farnesene synthase | 576 |
| 8 | ZoBBS - Amino acid Sequence \| Zingiber officinale - (S)- | 550 |
| | beta-bisabolene synthase | |
| 9 | CiCaSSy - Amino acid Sequence \| terpene synthases for | 553 |
| | Santalene; terpene synthases for myrcene (CiCaMS), | |
| | general Plant terpene synthases (TPSs) | |
| 10 | M1-alkB-F93S - Amino acid Sequence \| oxidation system | 297 |
| | alkB oxidase enzyme with point mutation of F93S | |
| 11 | M1-alkB-W189S - Amino acid Sequence \| oxidation system | 297 |
| | alkB oxidase enzyme with point mutation of W189S | |
| 12 | M1-alkB-rr-rrr - Nucleic acid Sequence \| alkane oxidation | 2657 |
| | system components a & c & d | |
| 13 | D000 \| Blank | — |
| 14 | MdAFS - Nucleic acid Sequence \| α-farnesene synthase | 2141 |
| 15 | ZoBBS - Nucleic acid Sequence \| Zingiber officinale - (S)- | 2012 |
| | beta-bisabolene synthase | |
| 16 | CiCaSSy - Nucleic acid Sequence \| terpene synthases for | 2021 |
| | Santalene; terpene synthases for myrcene (CiCaMS), | |
| | general Plant terpene synthases (TPSs) | |
| 17 | Synthetic construct - M1-alkB-F93S-rr-rrr - Nucleic acid | 2657 |
| | Sequence \| alkane oxidation system components a & c & d | |
| | with alkB point mutation of F93S | |
| 18 | Synthetic construct - M1-alkB-W189S-rr-rrr \| alkane | 2657 |
| | oxidation system components a & c & d with alkB point | |
| | mutation of W189S | |
| 19 | Synthetic construct - M1-alkB-rr - Nucleic acid Sequence | 1250 |
| | alkane oxidation system components a & c but not | |
| | component d i.e. without reductase | |
| 20 | M1 alkB DNA - Nucleic acid Sequence \| alkane 1-mono- | 894 |
| | oxygenase AlkB | |
| 21 | rubredoxin DNA - Nucleic acid Sequence \| rubredoxin | 159 |
| | peptide | |
| 22 | Rubredoxin reductase DNA - Nucleic acid Sequence \| | 1159 |
| | rubredoxin reductase | |
| 23 | Desaturase domain-containing protein \| Amino acid | 301 |
| | Sequence from Acinetobacter guillouiae | |
| 24 | Desaturase domain-containing protein from Acinetobacter | 301 |
| | sp. MYb10 \| Amino acid sequence | |
| 25 | Synthetic Amino acid Sequence \| alkane 1-mono- | 297 |
| | oxygenase AlkB \| Protein variant 1 | |
| 26 | Synthetic Amino acid Sequence \| alkane 1-mono- | 297 |
| | oxygenase AlkB \| Protein variant 2 | |
| 27 | Synthetic Amino acid Sequence \| alkane 1-mono- | 297 |
| | oxygenase AlkB \| Protein variant 3 | |
| 28 | Synthetic Amino acid Sequence \| alkane 1-mono- | 297 |
| | oxygenase AlkB \| Protein variant 4 | |
| 29 | Synthetic Amino acid Sequence \| alkane 1-mono- | 297 |
| | oxygenase AlkB \| Protein variant 5 | |
| 30 | Synthetic Amino acid Sequence \| alkane 1-mono- | 297 |
| | oxygenase AlkB \| Protein variant 6 | |
| 31 | Synthetic Amino acid Sequence \| alkane 1-mono- | 297 |
| | oxygenase AlkB \| Protein variant 7 | |
| 32 | Synthetic Amino acid Sequence \| alkane 1-mono- | 297 |
| | oxygenase AlkB \| Protein variant 8 | |
| 33 | Synthetic Amino acid Sequence \| alkane 1-mono- | 297 |
| | oxygenase AlkB \| Protein variant 9 | |
| 34 | Synthetic Amino acid Sequence \| alkane 1-mono- | 297 |
| | oxygenase AlkB \| Protein variant 10 | |
| 35 | Synthetic Amino acid Sequence \| alkane 1-mono- | 297 |
| | oxygenase AlkB \| Protein variant 11 | |
| 36 | Synthetic Amino acid Sequence \| alkane 1-mono- | 297 |
| | oxygenase AlkB \| Protein variant 12 | |
| 37 | Synthetic Amino acid Sequence \| alkane 1-mono- | 297 |
| | oxygenase AlkB \| Protein variant 13 | |
| 38 | Synthetic Amino acid Sequence \| alkane 1-mono- | 297 |
| | oxygenase AlkB \| Protein variant 14 | |
| 39 | Synthetic Amino acid Sequence \| alkane 1-mono- | 297 |
| | oxygenase AlkB \| Protein variant 15 | |
| 40 | Synthetic Amino acid Sequence \| alkane 1-mono- | 297 |
| | oxygenase AlkB \| Protein variant 16 | |
| 41 | Synthetic Amino acid Sequence \| alkane 1-mono- | 297 |
| | oxygenase AlkB \| Protein variant 17 | |
| 42 | Synthetic Amino acid Sequence \| alkane 1-mono- | 297 |
| | oxygenase AlkB \| Protein variant 18 | |
| 43 | CMR5c -alkB - Amino acid Sequence \| alkane 1-mono- | 296 |
| | oxygenase AlkB \| from AZC18544.1: protein C4K40_3156, | |
| | original organism Pseudomonas sp. | |
| 44 | \| SYNTHETIC GENE for the expression of the CMR5c -alkB | 1046 |
| | protein of SEQ ID NO: 43 and CMR5c rubredoxin known as | |
| | UniProt database entry A0A3G7A099 | |
| 45 | Amino Acid sequence of Pseudomonas sp. Strain CRM5c | 52 |
| | rubredoxin, UniProt database entry A0A3G7A099 | |

### Compounds, Compositions, Components

| | |
|---|---|
| Xbal restriction site | Restriction enzyme recognizes T\hat{ }CTAGA sites |
| Notl restriction site | Restriction enzyme recognizes GC\hat{ }GGCCGC sites |
| LB-agar | 10 g SELECT Peptone 140, 5 g SELECT Yeast Extract, 5 g |
| | sodium chloride, 12 g SELECT Agar |
| IPTG | Isopropyl β-D-1-thiogalactopyranoside |
| DCM | Dichloromethane |
| EcoRI | Restriction enzyme recognizes G\hat{ }AATTC sites |
| HindIII | Restriction enzyme recognizes A\hat{ }AGCTT sites |

### Vectors

| | |
|---|---|
| pET-DUET1 | Blank vector |
| | pET carries the pBR322-derived ColE1 replicon, lacl gene and |
| | ampicillin resistance gene. pET has a T7 promotor, is designed for |
| | the coexpression of two target genes and has a size of 5420 bp. |
| pET-M1-alkB- | Vector with inventive sequence |
| rr-rrr | |
| pUC57 | plasmid cloning vector in E. coli. 2,710 bp, Isolated from E. coli |
| | strain DH5 α by standard procedures. |

### Standard Methods and Instruments

| | |
|---|---|
| A600 | Cell growth profile observed at A600, |
| | i.e. absorbance at 600 nm |
| Gas | GC System: Model 7890A, Agilent |
| chromatography & | Mass selective detector: Model 5975C, |
| MS | Agilent scanning in the range 45-450 m/z |
| | Zebron ZB-5MS column (30 m × 0.25 mm, |
| | 0.25 μm thickness; Phenomenex) |

### Example 1: Constructs Expressing M1-alkB in E. coli

A construct was designed for expression in E. coli of M1-alkB (PM1_0216370), M1-rubredoxin (PM1_0216365), and M1-rubredoxin reductase (SEQ ID NO: 20 to 22 respectively). M1 alkB is localized at position 3472570-3471680 on contig 2 of the Pseudomonas M1 genome, and M1-rubredoxin is encoded immediately downstream of M1-alkB. The M1-rubredoxin reductase is not co-localized with M1-alkB, but is located on position 438956-437814 on contig 2 of the Pseudomonas M1 genome (Soares-Castro & Santos, 2014, Genome Biol. Evol. 7(1):1-17), and was identified by blast, using the rubredoxin reductase from Pseudomonas fluorescence (accession SUD34760.1) as bait.

Protein sequences of M1-alkB, M1-rubredoxin and M1-rubredoxin reductase are SEQ ID NO: 1, SEQ ID NO: 2, and SEQ ID NO: 3 respectively,

To express the M1 proteins in E. coli, open reading frames were codon optimized for expression in Rhodobacter sphaeroides in silico using standard tools, and pieces of artificial DNA were synthesized using a standard service provider, in which the M1-alkB, M1-rubredoxin, and the M1 rubredoxin reductase were encoded.

Synthetic nucleic acid sequence of SEQ ID NO: 4 provides for the M1-alkB-rubredoxin-rubredoxin reductase construct.

SEQ ID NO: 4 was cloned in vector pET-DUET1 using XbaI and NotI restriction sites, yielding pET-M1-alkB-rr-rrr for expression.

In an alternate Example 1a, the CMR5c protein shown in SEQ ID NO: 43, represents CMR5 oxidase/AlkB oxidase enzyme. A synthetic DNA sequence of SEQ ID NO. 44 encoded the protein of the SEQ ID NO: 43 (component a) as well as the rubredoxin peptide (component c) shown in SEQ ID NO: 45. SEQ ID NO. 44 has an overlapping region of “ATGA” covering both an ATG and a TGA, overlapping at TG. ATG in the overlap acts as the start codon for the rubredoxin peptide (component c) at the 888 bp location.

### Example 2: Testing the Ability of M1-alkB to Convert Myrcene and Beta Farnesene

The constructs pET-DUET-1 and pET-M1-alkB-rr-rrr were transformed to E. coli BL21-DE3, and selected on LB-agar with 1% glucose and 100 μg/ml ampicillin. Transformants harbouring pET-M1-alkB-rr-rrr and pET-DUET1 were inoculated in LB with 100 ug/ml ampicillin, and grown overnight at 37° C. and 225 rpm. Subsequently, cultures were diluted 1:20 in 10 ml 2×YT+Amp liquid medium, and incubated for 2 hours at 37° C. and 225 rpm, until the A600 was 0.6. Then 1 mM IPTG was added and cultures were incubated 2 h at 30° C. 225 rpm. Subsequently, 3 ml of the culture was mixed with 10 μl myrcene or 10 μl beta farnesene, or no product, closed in a 4 ml glass vial with screwcap and incubated for 3 hours at 225 rpm and 30° C. Subsequently, after extraction, the compounds were identified by METHOD A from the culture.

Myrcene conversion by E. coli BL21 with pET-DUET Comparative Strain (CS1) and pET-M1-alkB-rr-rrr i.e. Strain with artificial alkane oxidation system or Inventive Strain (IS1) is illustrated across FIG. 1a.

Oxidation products of myrcene were observed when bacterial cultures expressing pET-DUET1 (CS1) and pET-M1-alkB-rr-rrr (IS1) were compared. Notably, a compound eluting after 10.7 min (See compound 2, FIG. 1c) was observed, which was identified as myrcene aldehyde by its mass spectrum on comparison with the compound 1 of Literature (See FIG. 1b). In addition, several oxidation products of limonene were identified, which likely derive from a limonene contamination in the myrcene batch at 5%. Peak intensities of the limonene products were in the same range as the intensity from myrcene aldehyde, suggesting that limonene was preferred as a substrate over myrcene. The observation of formation of myrcene aldehyde shows that M1-alkB, when co-expressed with M1-rubredoxin and M1-rubredoxin reductase, can mediate oxidation of myrcene at its di-allylic position.

Referring to FIG. 2, when beta farnesene was used as a substrate for bacterial cultures expressing pET-DUET1 (CS1) and pET-M1-alkB-rr-rrr (IS1), also specific conversion products were observed, but much less pronounced (See FIG. 2). Products corresponded to hexadecenal (14.1 min), farnesol (14.8 min), denderalasin (15.3 min) and bicyclo-octanediol (16.0 min). In particular the observation of farnesol and denderalasin suggest that farnesene is used as a substrate by M1-alkB, but is modified at other positions than the diallylic position showing its ability for broad terpene oxidation.

### Example 3: Construction for Expression of alkB in Rhodobacter

The following pieces of synthetic DNA were synthesized by a standard service provider for custom DNA synthesis and were obtained cloned in vector pUC57.

DNA encoding protein sequences of SEQ ID NO: 6, SEQ ID NO: 7, SEQ ID NO: 8, SEQ ID NO: 9, are examples of alkane oxidation system components b: the (AaBFS) β-farnesene synthase, (MdAFS) α-farnesene synthase, (ZoBBS) Zingiber officinale—(S)-beta-bisabolene synthase, (CiCaSSy) Plant terpene synthase from Cinnamomum camphora. SEQ ID NO: 10 and SEQ ID NO: 11 are the sequence of the mutated alkane oxidation system components a, alkB (M1-alkB-F93S) with point mutation introduced by the inventors of F93S and (M1-alkB-W189S)alkane oxidation system with point mutation introduced by the inventors of W189S, respectively, as examples of further components a.

Nucleic acid sequences of SEQ ID NO: 5, SEQ ID NO: 12, SEQ ID NO: 13 corresponding to (AaBFS) Artemisia annua β-farnesene synthase, (M1-alkB-rr-rrr)alkane oxidation system components a, c and d, and (D000) blank sequence respectively were synthesized by a standard service provider.

These sequences were delivered as part of pUC57.

Subsequently, plasmids pUC57-AaBFS was digested using restriction enzymes EcoRI and HindIII. Plasmids pUC57-M1-alkB-rr-rrr and pUC57-D000 were each digested with BamHI and HindIII, and were combined with a BamHI-EcoRI digested p-m-LPppa-CiCaSSy-mpmii alt (described in US2020/0010822) in a ligation reaction, according to standard procedures. The ligation was transformed into E. coli S17-1, and selected on LB with 100 ug/ml Neomycin.

Resulting E. coli S17-1 colonies harboured the plasmid pBBR-AaBFS-M1-alkB-rr, or pBBR-MVA-AaBFS-D000. These plasmids were conjugated to Rhodobacter sphaeroides strain Rs265-9c using methods disclosed in the international patent application WO2011074954 (see pages 64 to 67), and trans-conjugants were selected using plates with Ra medium and 100 ug/ml neomycin. Rhodobacter colonies were selected which harboured the plasmid pBBR-MVA-AaBFS-M1-alkB-rr, or pBBR-MVA-AaBFS-D000. Resulting strains were named after their plasmids.

Strain Rs265-9c-pBBR-MVA-AaBFS-M1-alkB-rr-rrr (IS2)—carrying the a beta-Farnese synthase, the oxidase enzyme alkB and a rubredoxin and a rubredoxin reductase- and as control Rs265-9c-pBBR-MVA-AaBFS-D000 (CS2) were cultivated in 20 ml RS102 medium, using 2 ml n-dodecane as an overlay, basically as described in WO2018160066A1. The n-dodecane layer was harvested after 72 h of cultivation, and was analysed by GC-MS basically as described in WO2018160066A1 (See FIG. 3a).

The n-dodecane of culture Rs265-9c-pBBR-MVA-AaBFS-D000 (CS2) contained beta-farnesene as the dominant peak, which eluted at 16.81 min. Dodecane from Rs265-9c-pBBR-MVA-AaBFS-M1-alkB-rr-rrr (IS2) displayed an additional peak at 22.23 min, which was absent from (CS2) Rs265-9c-pBBR-MVA-AaBFS-D000 (See FIG. 3b). Comparison of the mass spectrum and retention time of the peak eluting at 22.23 min and the mass spectrum and retention time of a standard of sinensal (Aroma Uden), revealed that this peak is representing beta sinensal (See FIG. 3c for (IS2) Rs265-9c-pBBR-MVA-AaBFS-M1-alkB-rr-rrr based and See FIG. 3d for the Sinensal standard).

A quantitative analysis revealed that 15.2 g farnesene per kg dodecane was produced, while 0.38 g sinensal per kg dodecane was produced.

### Example 4

It was tested if the M1-alkB module would be able to oxidize alpha farnesene to alpha sinensal. First, a synthetic construct expressing alpha farnesene synthase from Malus domesticus was ordered from standard service provider, and was delivered in plasmid pUC57).

Nucleic acid sequence of SEQ ID NO: 14 corresponds to the nucleic acid for (MdAFS) a-farnesene synthase.

Subsequently, plasmids pUC57-MdAFS was digested using restriction enzymes EcoRI and HindIII. Plasmids pUC57-M1-alkB-rr-rrr and pUC57-D000 were each digested with BamHI and HindIII, and were combined with a BamHI-EcoRI digested p-m-LPppa-CiCaSSy-mpmii alt pDEST4 in a ligation reaction, according to standard procedures. The ligation was transformed into E. coli S17-1, and selected on LB with 100 ug/ml Neomycin. Resulting E. coli S17-1 colonies harboured the plasmid pBBR-MVA-MdAFS-M1-alkB-rr-rrr, or pBBR-MVA-MdAFS-D000. These plasmids were conjugated to Rhodobacter sphaeroides strain Rs265-9c using standard procedures for example as in in the international patent application WO2011074954, and trans-conjugants were selected using plates with Ra medium and 100 ug/ml neomycin. Rhodobacter colonies were selected which harboured the plasmid pBBR-MVA-MdAFS-M1-alkB-rr-rrr, or pBBR-MVA-MdAFS-D000. Resulting strains were named after their plasmids.

Strain (IS3) Rs265-9c-pBBR-MVA-MdAFS-M1-alkB-rr-rrr and (CS3) Rs265-9c-pBBR-MVA-MdAFS-D000 without the enzyme were cultivated in 20 ml RS102 medium, using 2 ml n-dodecane as an overlay, basically as described in WO2018160066A1. The n-dodecane layer was harvested after 72 h of cultivation, and was analysed by GC-MS basically as described in WO2018160066A1.

The n-dodecane of control culture (CS3) Rs265-9c-pBBR-MVA-MdAFS-D000 contained alpha-farnesene as the dominant peak, which eluted at 19.28 min. Dodecane from (IS3) Rs265-9c-pBBR-MVA-AaBFS-M1-alkB-rr-rrr displayed an additional peak at 24.88 min, which was absent from (CS3) Rs265-9c-pBBR-MVA-MdAFS-M1-D000 (see FIG. 4). Comparison of the mass spectrum and retention time of the peak eluting at 24.883 min and the mass spectrum and retention time of a standard of sinensal (Aroma Uden), revealed that this peak is representing alpha sinensal.

### Example 5

It was tested if the M1-alkB module would be able to oxidize other sesquiterpenes to aldehydes. First, a synthetic construct expressing beta bisabolene synthase (ZoBBS) from Zingiber officinale (BAI67934.1), and a synthetic construct expressing santalene synthase (CiCaSSy) from Cinnamomum camphora (QNV69588.1) was synthesized by a standard service provider and delivered in plasmid pUC57).

Nucleic acid sequence of SEQ ID NO: 15 and SEQ ID NO: 16 correspond to Zingiber officinale—(S)-beta-bisabolene synthase (ZoBBS) and Plant terpene synthases from Cinnamomum camphora (CiCaSSy).

Subsequently, plasmids pUC57-ZoBBS and pUC57-CiCaSSy were digested using restriction enzymes EcoRI and HindIII. Plasmids pUC57-M1-alkB-rr-rrr and pUC57-D000 were each digested with BamHI and HindIII, and were combined with a BamHI-EcoRI digested p-m-LPppa-CiCaSSy-mpmii alt in a ligation reaction, according to standard procedures. The ligations were transformed into E. coli S17-1, and selected on LB with 100 ug/ml Neomycin.

Resulting E. coli S17-1 colonies harboured the plasmid pBBR-MVA-ZoBBS-M1-alkB-rr-rrr, or pBBR-MVA-ZoBBS-D000, and BBR-MVA-CiCaSSy-M1-alkB-rr-rrr, or pBBR-MVA-CiCaSSy-D000. These plasmids were conjugated to Rhodobacter sphaeroides strain Rä265-9c using standard methods, and trans-conjugants were selected using plates with Ra medium and 100 ug/ml neomycin. Rhodobacter colonies were selected which harboured the plasmid pBBR-MVA-ZoBBS-M1-alkB-rr-rrr, or pBBR-MVA-ZoBBS-D000, and BBR-MVA-CiCaSSy-M1-alkB-rr-rrr, or pBBR-MVA-CiCaSSy-D000. Resulting strains were named after their plasmids.

Strain (IS4) Rs265-9c-pBBR-MVA-ZoBBS-M1-alkB-rr-rrr and as control (CS4) Rs265-9c-pBBR-MVA-ZoBBS-D000, (IS5) Rs265-9c-pBBR-MVA-CiCaSSy-M1-alkB-rr-rrr and as control (CS5) Rs265-9c-pBBR-MVA-CiCaSSy-D000 were cultivated in 20 ml RS102 medium, using 2 ml n-dodecane as an overlay, basically as described in WO2018160066A1. The n-dodecane layer was harvested after 72 h of cultivation, and was analysed by GC-MS basically as described in WO2018160066A1.

Referring to FIG. 6, the n-dodecane of culture Rs265-9c-pBBR-MVA-CiCaSSy-D000 contained beta-alpha santalene, bergamotene and beta santalene as the dominant peaks, at retention time 17.23 min and 18.23 min, respectively. Dodecane from Rs265-9c-pBBR-MVA-CiCaSSy-M1-alkB-rr-rrr displayed additional peaks at 23.75 min and 24.66 min, which were absent from (CS5) Rs265-9c-pBBR-MVA-CiCaSSy-M1-D000. Comparison of the mass spectrum and retention time of the peaks eluting at 23.75 min and 24.66 min to the NIST library and to a sandalwood standard revealed that they correspond to trans alpha santalol and trans beta santalol.

Referring to FIG. 5, the n-dodecane of culture (CS4) Rs265-9c-pBBR-MVA-ZoBBS-D000 contained beta-bisabolene as the dominant peak, which eluted at 19.37 min. Dodecane from (IS4) Rs265-9c-pBBR-MVA-ZoBBS-M1-alkB-rr-rrr displayed an additional peak at 25.16 min, which was absent from (CS4) Rs265-9c-pBBR-MVA-ZoBBS-M1-D000. GC MS analysis revealed that this peak is representing lanceol-aldehyde, an allylic oxidation product from beta bisabolene.

### Example 6

### Effect of Mutation of M1-alkB

Two mutants of the M1-alkB protein were designed, leading to an M1-alkB-F93S variant, and an M1-alkB-W189S. Expression constructs for these two mutants were synthesized by a standard service provider.

Subsequently, plasmid pUC57-AaBFS was digested using restriction enzymes EcoRI and HindIII. Plasmids pUC57-M1-alkB-F93S-rr-rrr (which carried the sequence of SEQ ID NO: 17) and pUC57-M1-alkB-W189S-rr-rrr (which carried the sequences of SEQ ID NO: 18) were each digested with BamHI and HindIII, and were combined with a BamHI-EcoRI digested p-m-LPppa-CiCaSSy-mpmii alt (described in US2020/0010822) in a ligation reaction, according to standard procedures. The ligation was transformed into E. coli S17-1, and selected on LB with 100 ug/ml Neomycin.

Resulting E. coli S17-1 colonies harboured the plasmid pBBR-AaBFS-M1-alkB-F93S-rr-rrr, or pBBR-MVA-AaBFS-M1-alkB-W189S-rr-rrr. These plasmids were conjugated to Rhodobacter sphaeroides strain Rs265-9c using standard methods, and trans-conjugants were selected using plates with Ra medium and 100 ug/ml neomycin. Rhodobacter colonies were selected which harboured the plasmid pBBR-MVA-AaBFS-M1-alkB-F93S-rr-rrr, or pBBR-MVA-AaBFS-M1-alkB-W189S-rr-rrr. Resulting strains were named after their plasmids.

Strain (IS6a) Rs265-9c-pBBR-MVA-AaBFS-M1-alkB-F93S-rr-rrr and (IS6b) Rs265-9c-pBBR-MVA-AaBFS-M1-alkB-W189S-rr-rrr were cultivated in 20 ml RS102 medium, using 2 ml n-dodecane as an overlay, as described in example 3. The n-dodecane layer was harvested after 72 h of cultivation, and was analysed by GC-MS.

A quantitative analysis revealed that for both Strain (IS6a) Rs265-9c-pBBR-MVA-AaBFS-M1-alkB-F93S-rr-rrr and (IS6b) Rs265-9c-pBBR-MVA-AaBFS-M1-alkB-W189S-rr-rrr 16.0 g farnesene per kg dodecane was produced. Strain (IS6a) Rs265-9c-pBBR-MVA-AaBFS-M1-alkB-F93S-rr-rrr produced 0.81 g sinensal per kg dodecane, while (IS6b) Rs265-9c-pBBR-MVA-AaBFS-M1-alkB-W189S-rr-rrr produced 0.51 g sinensal per kg dodecane was produced. Thus, both mutations in M1-alkB seem to contribute to a higher sinensal production.

Nucleic acid sequence of SEQ ID NO: 17 and SEQ ID NO: 18 correspond to alkane oxidation system components a & c with point mutation of F93S in a, i.e. M1-alkB-F93S-rr-rrr and alkane oxidation system components a & c with point mutation of W189S in a, i.e. M1-alkB-W189S-rr-rrr

TABLE 1
Transformed Strains substrates and products in Examples 2 to 7.

| | Comparative | | | Oxidised terpene |
|---|---|---|---|---|
| | Strains (CS) | | | products |
| | and | | | (time of peak |
| Example | Inventive | | | in mins) (if |
| no. | Strains (IS). | Strain details | Substrate | multiple products) |
| Example | CS1 | E. coli BL21- | myrcene | — |
| 2 | | DE3 with pET- | | |
| | | DUET | | |
| Example | IS1 | E. coli BL21- | myrcene | myrcene aldehyde, |
| 2 | | DE3 | | limonene (due to limonen |
| | | with pET-M1- | | contamination) |
| | | alkB-rr-rrr | | |
| Example | IS1 | E. coli BL21- | beta | hexadecenal (14.1 min), |
| 2 | | DE3 with pET- | farnescene | farnesol (14.8 min), |
| | | M1-alkB-rr-rrr | | denderalasin (15.3 min) |
| | | | | and bicyclo-octanediol |
| | | | | (16.0 min). |
| Example | CS2 | Rs265-9c- | beta- | |
| 3 | | pBBR-MVA- | farnesene | |
| | | AaBFS-D000 | | |
| Example | IS2 | Rs265-9c- | beta- | sinensal (beta) |
| 3 | | pBBR-MVA- | farnesene | |
| | | AaBFS-M1- | | |
| | | alkB-rr-rrr | | |
| Example | CS3 | Rs265-9c- | alpha- | — |
| 4 | | pBBR-MVA- | farnesene | |
| | | MdAFS-D000 | | |
| Example | IS3 | Rs265-9c- | alpha- | alpha sinensal |
| 4 | | pBBR-MVA- | farnesene | |
| | | MdAFS-M1- | | |
| | | alkB-rr-rrr | | |
| Example | CS4 | Rs265-9c- | beta- | |
| 5 | | pBBR-MVA- | bisabolene | |
| | | ZoBBS-D000 | | |
| Example | IS4 | Rs265-9c- | beta- | lanceol-aldehyde |
| 5 | | pBBR-MVA- | bisabolene | (25.16 min) |
| | | ZoBBS-M1- | | |
| | | alkB-rr-rrr | | |
| Example | CS5 | Rs265-9c- | beta-alpha | — |
| 5 | | pBBR-MVA- | santalene, | |
| | | CiCaSSy-D000 | bergamotene | |
| | | | (17.23 min) | |
| | | | and beta | |
| | | | santalene | |
| | | | (18.23 min) | |
| Example | IS5 | Rs265-9c- | beta-alpha | Trans alpha |
| 5 | | pBBR-MVA- | santalene, | santalol |
| | | CiCaSSy-M1- | bergamotene | (23.75 min), |
| | | alkB-rr-rrr | (17.23 min) | trans beta |
| | | | and beta | santalol |
| | | | santalene | (24.66) |
| | | | (18.23 min) | |
| Example | IS6a | Rs265-9c- | beta- | sinensal |
| 6 | | pBBR-MVA- | farnesene | |
| | | AaBFS-M1- | | |
| | | alkB-F93S-rr- | | |
| | | rrr | | |
| Example | IS6b | Rs265-9c- | beta- | sinensal |
| 6 | | pBBR-MVA- | farnesene | |
| | | AaBFS-M1- | | |
| | | alkB-W189S- | | |
| | | rr-rrr | | |
| Example | IS7a | Rs265-9c- | beta | sinensal |
| 7 | | pBBR-MVA- | farnesene | |
| | | AaBFS-M1- | | |
| | | alkB-F93S-rr- | | |
| | | rrr - 2 | | |
| Example | IS7b | Rs265-9c- | beta | sinensal |
| 7 | | pBBR-MVA- | farnesene | |
| | | AaBFS-M1- | | |
| | | alkB-F93V-rr- | | |
| | | rrr | | |
| Example | IS7c | Rs265-9c- | beta | sinensal |
| 7 | | pBBR-MVA- | farnesene | |
| | | AaBFS-M1- | | |
| | | alkB-F93G-rr- | | |
| | | rrr | | |
| Example | IS7d | Rs265-9c- | beta | sinensal, |
| 7 | | pBBR-MVA- | farnesene | no production |
| | | AaBFS-M1- | | of farnesol |
| | | alkB-F93A-rr- | | |
| | | rrr | | |
| Example | IS8 | Rs265-9c- | beta | sinensal |
| 8 | | pBBR-AaBFS- | farnesene | |
| | | M1-alkB-rr-rrr | | |

### Example 7: Point Mutations

Position 93 in alkB protein encodes a phenylalanine. The F93 position was changed into a serine, and the M1-alkB was introduced in combination with a beta farnesene synthase, a rubredoxin and optionally a rubredoxin reductase. The point mutation was associated with an improved productivity of beta sinensal. The point mutation was also associated with the formation of farnesol as a side product as well as also an oxidised terpene end product.

Point mutations observed in position 93 include F93V, F931, F93A, F93R, F93W, F93T, F93G. From these, F93A produced the highest amount of beta sinensal, when combined with beta farnesene synthase. With the F93A mutant, no formation of farnesol was observed.

TABLE 2
F93 point mutations.

| | Codon | Beta | Beta | Percentage | Ratio of |
|---|---|---|---|---|---|
| Position | coding | sinensal | farnesene | Sinensal | Sinensal |
| 93rd | residue | (g/kg | (g/kg | to | to |
| residue | 93 | dodecane) | dodecane) | Farnescene | Farnescene |
| R | CGC | 0 | 18.45 | — | — |
| I | ATC | 0.11 | 18.11 | 0.6% | 1:160 |
| W | TGG | 0.19 | 18.8 | 1.01% | 1:98 |
| T | ACC | 0.45 | 16.27 | 2.77% | 1:36 |
| F | TTC | 0.6 | 18.46 | 3.25% | 1:30 |
| S | TCG | 0.87 | 17.35 | 5.01% | 1:20 |
| V | GTG | 0.93 | 16.38 | 5.68% | 1:17.6 |
| G | GGC | 0.92 | 16.08 | 5.72% | 1:17.4 |
| A | GCC | 1.1 | 15.81 | 6.96% | 1:14 |

Table 2 above provides for the amount of Beta Farnesene and Beta Sinensal obtained by the point mutations at 93rd residue in the alkB protein over a period of 72 hours. Originally the 93rd residue is “F”. The point mutation of F93S, F93V, F93G, F93A associated with Strains (IS7a) i.e. Rs265-9c-pBBR-MVA-AaBFS-M1-alkB-F93S-rr-rrr-2, (IS7b) i.e. Rs265-9c-pBBR-MVA-AaBFS-M1-alkB-F93V-rr-rrr, (IS7c) i.e. Rs265-9c-pBBR-MVA-AaBFS-M1-alkB-F93G-rr-rrr and (IS7d) i.e. Rs265-9c-pBBR-MVA-AaBFS-M1-alkB-F93A-rr-rrr were associated with improved Beta Farnesene and Beta Sinensal production compared to original alkB with F in 93rd position.

However, the point mutation of F93R, F931, F93W, F93T were not associated with improved production of oxidised terpene product, the improvement being relative to the original 93rd residue i.e. “F”.

### Example 8: Sinensal Production in the Absence of Component d and without MVA

Similar to example 3 above a strain IS8 carrying the construct Rs265-9c-pBBR-AaBFS-M1-alkB-rr-rrr was tested. The difference to example 3 was, that the strain IS8 did not comprise the rubredoxin reductase or another exogenous component d and also did not comprise the heterologous MVA pathway. The resulting amounts of sinensal produced were comparable to the sinensal production of IS2, demonstrating that components a, b and c alone were sufficient for the production of the oxidised terpene product sinensal.

### Example 9

Similar to the testing of the alkB construct in example 3 above, the construct of SEQ ID NO: 44 was tested. The results showed that the CMR5c protein was also suited as an oxidase enzyme in the alkane oxidation system.

### Example 10

Based on the method for preparing a variant polypeptide as describe hereinabove, variant polypeptides of SEQ ID NO. 25 to 42 were obtained. FIG. 7a &7b shows the alignment of the polypeptides of the invention with conserved amino acids at the positions indicated. The polypeptide variants of the invention comprise conserved amino acids at the positions indicated in FIGS. 7a & 7b. Conserved amino acid positions are indicated in figures by letters in white font on black background

### Example 11

Based on the methods disclosed hereinabove, further experimental analysis were done with strain of the aforementioned Rhodobacter sp. One strain was developed such that the strain carried all components a, b, c, and d of the alkane oxidation system. Another strain was developed such that the strain carried only component a (oxidase enzyme) and component b (terpene synthase) and did not carry component c (rubredoxin) and component d (rubredoxin reductase). Both strains did show oxidation of beta sinensal from beta farnesene.

Table 3 below provides for the amount of Beta Farnesene and Beta Sinensal for the strains after a period of 72 hours.

TABLE 3

| | Beta | Beta | Percentage | Ratio of |
|---|---|---|---|---|
| | sinensal | farnesene | Sinensal | Sinensal |
| | (g/kg | (g/kg | to | to |
| Strain | dodecane) | dodecane) | Farnescene | Farnescene |
| Strain with all components | 0.7 | 15.2 | 4.6% | Ratio 1:21 |
| a, b, c and d | | | | |
| Strain with a and b and | 0.1 | 20.5 | 0.49% | Ratio 1:205 |
| without c and d | | | | |

1.-15. (canceled)

16. An artificial alkane oxidation system, the system comprising components:
a. an oxidase enzyme; and
b. one or more enzymes to provide one or more alkanes;
and
c. optionally an electron transfer compound suitable to transfer at least one electron to the oxidase enzyme; and
d. optionally an electron transfer compound regeneration enzyme suitable to reduce the electron transfer compound from oxidized state;
wherein the oxidase enzyme is an amino acid sequence with sequence identity of at least 70% of any one of SEQ ID NO: 1, 10, 11, 23, 24, 25, 26, 27, 28, 29, 30, 31, 32, 33, 34, 35, 36, 37, 38, 39, 40, 41, 42, 43, or a fragment thereof.

17. The artificial alkane oxidation system of claim 16, wherein the one or more alkanes has at least five carbon atoms, or has at least one five carbon building block, or
wherein the component b is at least one terpene synthase protein and the one or more alkanes is a terpene.

18. The artificial alkane oxidation system of claim 16, wherein the electron transfer compound is an electron transfer protein, or/and the electron transfer compound regeneration enzyme is an electron transfer protein reductase.

19. A synthetic nucleic acid sequence with a sequence identity of at least 70% with any one of SEQ ID NO: 4, 12, 17, 18, 19, or 44, wherein the synthetic nucleic acid sequence comprises a nucleic acid sequence encoding the oxidase enzyme as defined in claim 16, and optionally an electron transfer protein and/or an electron transfer protein reductase.

20. A kit for the artificial alkane oxidation system, the kit comprising:
a first isolated nucleic acid sequence encoding an oxidase enzyme as defined in claim 16; and
a second isolated nucleic acid sequence encoding an electron transfer protein; and
optionally a third nucleic acid sequence encoding an electron transfer protein reductase, wherein the third nucleic acid sequence may be fused to the first and/or second nucleic acid sequence.

21. An expression cassette comprising:
1) a synthetic nucleic acid sequence with a sequence identity of at least 70% with any one of SEQ ID NO: 4, 12, 17, 18, 19, or 44, wherein the synthetic nucleic acid sequence comprises a nucleic acid sequence encoding the oxidase enzyme as defined in claim 16, and optionally an electron transfer protein and/or an electron transfer protein reductase; or
2) the nucleic acid sequence encoding the alkane oxidation system of claim 16; or
3) the nucleic acid sequence(s) of the alkane oxidation system including:
a. a nucleic acid sequence for an oxidase enzyme:
having a sequence identity of at least 70% with SEQ ID NO: 20, or
encoding for an oxidase enzyme with a sequence identity of at least 70% with any one of SEQ ID NO: 1, 10, 11, 23, 24, 25, 26, 27, 28, 29, 30, 31, 32, 33, 34, 35, 36, 37, 38, 39, 40, 41, 42, or 43; and
b. a nucleic acid sequence for a rubredoxin peptide:
having a sequence identity of at least 70% with SEQ ID NO: 21, or
encoding for a rubredoxin of SEQ ID NO: 2 or SEQ ID NO: 45, and optionally
c. a nucleic acid sequence for a rubredoxin reductase peptide:
having a sequence identity of at least 70% with of SEQ ID NO: 22, or
encoding for a rubredoxin reductase peptide of SEQ ID NO: 3.

22. A method for oxidation of at least one alkane substrate, the method comprising:
a. providing
i. the artificial alkane oxidase system of claim 16, and/or expression of a synthetic nucleic acid sequence with a sequence identity of at least 70% with any one of SEQ ID NO: 4, 12, 17, 18, 19, or 44, wherein the synthetic nucleic acid sequence comprises a nucleic acid sequence encoding the oxidase enzyme as defined in claim 16, and optionally an electron transfer protein and/or an electron transfer protein reductase, or an expression cassette comprising:
(a) a synthetic nucleic acid sequence with a sequence identity of at least 70% with any one of SEQ ID NO: 4, 12, 17, 18, 19, or 44, wherein the synthetic nucleic acid sequence comprises a nucleic acid sequence encoding the oxidase enzyme as defined in claim 16, and optionally an electron transfer protein and/or an electron transfer protein reductase
(b) the nucleic acid sequence encoding the alkane oxidation system of claim 16; or
(c) the nucleic acid sequence(s) of the alkane oxidation system including:
a nucleic acid sequence for an oxidase enzyme having a sequence identity of at least 70% with SEQ ID NO: 20, or encoding for an oxidase enzyme with a sequence identity of at least 70% with any one of SEQ ID NO: 1, 10, 11, 23, 24, 25, 26, 27, 28, 29, 30, 31, 32, 33, 34, 35, 36, 37, 38, 39, 40, 41, 42, 43; and
a nucleic acid sequence for a rubredoxin peptide having a sequence identity of at least 70% with SEQ ID NO: 21, or encoding for a rubredoxin of SEQ ID NO: 2 or SEQ ID NO: 45, and optionally
a nucleic acid sequence for a rubredoxin reductase peptide having a sequence identity of at least 70% with of SEQ ID NO: 22, or encoding for a rubredoxin reductase peptide of SEQ ID NO: 3, and/or
ii. expression of a nucleic acid sequence encoding an oxidase enzyme with an amino acid sequence with a sequence identity of at least 70% with any one of SEQ ID NO: 1, 10, 11, 23, 24, 25, 26, 27, 28, 29, 30, 31, 32, 33, 34, 35, 36, 37, 38, 39, 40, 41, 42, or 43 or a fragment thereof or a variant thereof, and/or
iii. wherein the expression cassette produces the encoded polypeptides; and
iv. optionally, one or more alkane substrates,
for oxidation of the at least one alkane substrate;
b. allylic oxidation of the at least one alkane substrate to produce at least one oxidised alkane product; and
c. optionally extracting the at least one oxidised alkane product.

23. The method of claim 22, wherein the at least one alkane substrate is selected from diterpenes, monoterpenes or sesquiterpenes, alpha farnesene, beta farnesene, alpha bisabolene, beta bisabolene, alpha bergamotene, beta bergamotene, alpha santalene, beta santalene, valencene, alpha guaiene, monoterpene geraniol, nerol monoterpenes, geranylactone, linalyl acetate, limonene, beta-pinene, or combination thereof.

24. The method of claim 22, wherein the at least one oxidized alkane product is selected from an alcohol and/or an aldehyde, wherein the alcohol and/or the aldehyde is myrcene aldehyde, alpha sinensal, beta sinensal, trans alpha santalol, trans beta santalol, lanceol-aldehyde, nootkatone, vetivone, rotundone, rebaudiosides, 8-hydroxygeraniol, 8-hydroxynerol, 9,10-epoxygeranylacetone, hexadecenal, farnesol, denderalasin, bicyclo-octanediol, oxidized alpha guaiene, oxidized beta guaiene, or a combination thereof.

25. A method of producing at least one oxidised alkane product by oxidation of an alkane substrate including the steps a to c of claim 22.

26. A non-human host cell comprising the artificial alkane oxidation system of claim 16.

27. A composition produced by the method of claim 22, wherein the composition comprises the at least one oxidised alkane product selected from myrcene aldehyde, alpha sinensal, beta sinensal, trans alpha santalol, trans beta santalol, lanceol-aldehyde, nootkatone, vetivone, rotundone, rebaudiosides, 8-hydroxygeraniol, 8-hydroxynerol, 9,10-epoxygeranylacetone, hexadecenal, farnesol, denderalasin bicyclo-octanediol, oxidized alpha guaiene or oxidized beta guaiene, or combination thereof; and optionally, one or more terpene substrates.

28. A composition produced by a non-human host cell comprising the artificial alkane oxidation system of claim 16 and at least one oxidised terpene product selected from myrcene aldehyde, alpha sinensal, beta sinensal, trans alpha santalol, trans beta santalol, lanceol-aldehyde, nootkatone, vetivone, rotundone, rebaudiosides, 8-hydroxygeraniol, 8-hydroxynerol, 9,10-epoxygeranylacetone, hexadecenal, farnesol, denderalasin bicyclo-octanediol, oxidized alpha guaiene or oxidized beta guaiene, or combination thereof; and optionally, one or more terpene substrates.

29. A fermentation composition comprising:
the non-human host cell of claim 26, cultured in a culture medium; and
the at least one oxidised alkane product produced from the non-human host cell as a major compound,
wherein the fermentation composition includes the alkane substrate and optionally one or more co-products are minor compounds.

30. A method of fermentative production of at least one oxidised alkane product, the method comprising:
providing a non-human host cell with the oxidase enzyme system as claimed in claim 16, optionally providing at least one alkane synthase to the non-human host cell for alkane substrate production;
culturing the non-human host cell in a culture medium under conditions suitable for the non-human host cell to produce the encoded polypeptides in active form;
optionally, producing one or more alkane substrates and/or providing one or more alkane substrates to the non-human host cell; and
producing at least one oxidised alkane product; and
optionally, purifying the at least one oxidised alkane product.

31. A method for preparing a variant polypeptide having oxidase enzyme activity, the method comprising steps of:
a) selecting a synthetic nucleic acid sequence with a sequence identity of at least 70% with any one of SEQ ID NO: 4, 12, 17, 18, 19, or 44, wherein the synthetic nucleic acid sequence comprises a nucleic acid sequence encoding the oxidase enzyme as defined in claim 16, and optionally an electron transfer protein and/or an electron transfer protein reductase, or a nucleic acid encoding an amino acid sequence with a sequence identity of at least 70% with any one of SEQ ID NO: 1, 10, 11, 23, 24, 25, 26, 27, 28, 29, 30, 31, 32, 33, 34, 35, 36, 37, 38, 39, 40, 41, 42, 43, or a fragment thereof;
b) modifying the selected nucleic acid to obtain at least one mutant nucleic acid;
c) transforming the non-human host cells with the mutant nucleic acid sequence to express the oxidase enzyme encoded by the mutant nucleic acid sequence;
d) screening the oxidase enzyme for at least one modified property in addition to the functionality of the oxidase enzyme encoded by the selected nucleic acid of a); and,
e) optionally, repeating the process steps (a) to (d) to obtain an oxidase enzyme with a desired variant functionality;
f) optionally, isolating the mutant nucleic acid sequence from the transformed non-human host cell obtained in step (c).
